SEQUENCE LISTING (50 sequences)

SEQ ID NO: 1 - DNA, 1536 bp, Vesicular stomatitis virus
  source 1..1536: mol_type = genomic DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 2 - protein, 511 aa, Vesicular stomatitis virus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - DNA, 1539 bp, Vesiculovirus cocal
  source 1..1539: mol_type = genomic DNA
atgaattttctgctccttacctttatagtgctcccactctgtagtcacgcgaaatttagtattgtattcccccaatctcaaaagggcaattggaagaacgtaccaagcagctaccactattgccccagtagttccgaccagaattggcataatgacctcttggggatcactatgaaggtgaagatgccagcgacgcacaaggctatccaagccgacggctggatgtgccatgcagcgaaatggattacgacatgcgattttcgctggtacgggccaaagtacataacacacagcatacatagtatacaacctacttccgagcaatgcaaggagagtattaagcaaactaagcaggggacctggatgagcccgggatttccgccacagaactgcggttatgccactgtgacggatagtgtagctgtcgttgtccaagcaaccccgcatcacgtcctcgtcgatgagtacacgggtgaatggattgattctcaattccctaacggaaaatgtgaaacagaggaatgtgagacagttcataattcaacggtgtggtattcagactacaaagttacaggcttgtgtgatgcgacgttggtcgacacagaaatcacgttctttagtgaagatggcaaaaaggaatccatcggaaaaccaaacacaggttaccgcagcaacgcatttgcttatgaaaaaggggacaaggtctgtaaaatgaattattgtaaacacgcgggtgtgagactcccatccggcgtctggtttgaatttgtggaccaggatgtctatgcggcggctaagcttcctgagtgcccagtcggtgctactatcagtgcgcccacacagacatcagttgatgtcagcctcatactggatgtcgaacgtattctggattactccctttgccaagagacatggagtaagatcaggtccaagcagcccgtaagcccggtagacctgtcctatctcgcaccgaagaaccctggtactggacctgcttttacaatcataaatggaacccttaaatatttcgaaactcgctacatacgcattgacatagacaacccgataatctccaaaatggtcggaaaaataagtggctcacaaacggaggccgaactttggacagaatggtttccttatgaaggggtggagatcggacccaacggaattttgaagacaccaacaggatacaagtttccactgtttatgattggacatggcatgttggattctgaccttcacaaaacgtcccaagcagaagtattcgagcacccacatctcgccgaagcgccaaagcagcttcctgaagaggaaactcttttctttggtgacacggggattagtaagaatcccgtagaactgattgaggggtggtttagttcctggaagtcaacagtcgtaaccttttttttcgcaataggtgtgtttattttgctctacgtggtcgccaggatagttatcgccgtccggtaccgctatcaagggtctaataataagcggatatacaacgacatcgagatgagtcgatttcgcaaatga

SEQ ID NO: 4 - protein, 512 aa, Vesiculovirus cocal
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 5 - DNA, 3747 bp, Alphavirus chikungunya
  source 1..3747: mol_type = genomic DNA
atggagtttatccctacacaaacattttataacagaaggtatcagcctagaccttggacccccagacctacaattcaagtgattagacctaggcctagacctcaaaggcaagccggacagctcgcccagctcatctccgctgtcaacaaactgaccatgagggccgtcccccaacagaagcctagaaaaaacagaaagaacaaaaaacagaaacagaagcagcaagccccccagaataacacaaatcagaagaagcagccccctaaaaagaagcccgcccagaagaagaagaagcccggaagaagagagagaatgtgtatgaaaatcgaaaatgactgcattttcgaggtcaaacacgaaggcaaggtgaccggctacgcttgtctggtgggcgacaaggtgatgaagcccgcccacgtgaagggcacaatcgacaacgccgatctggccaagctggcctttaagaggtccagcaagtacgacctcgaatgtgcccagatccccgtgcacatgaagtccgatgccagcaagttcacacacgagaagcccgaaggctactataactggcaccatggagccgtgcagtatagcggaggaagatttaccattcccaccggagccggaaaacccggcgacagcggaaggcccattttcgacaacaagggcagagtggtcgccatcgtgctcggaggcgctaacgaaggagctagaacagctctctccgtggtgacatggaacaaagacatcgtcaccaagatcacacccgagggagctgaggaatggagcctcgccattcccgtgatgtgtctgctggctaacacaacattcccttgcagccagcctccttgcattccttgttgctacgagaaggagcccgaagagacactgaggatgctcgaggataacgtcatgagacccggctactatcagctgctgcaagcctctctgacatgctccccccataggcagagaagatccaccaaggataactttaacgtgtataaggccacaagaccctacctcgctcattgccccgactgcggcgagggccatagctgtcacagccccgtggctctggaaaggattaggaacgaggctaccgatggaacactgaagatccaagtctctctgcagatcggcattggcacagacgattcccacgattggacaaagctgagatacatggacaatcacatccccgccgatgccggaagagccggactctttgtgaggacaagcgctccttgcaccatcaccggcacaatgggccattttatcctcgccagatgtcccaaaggcgagacactgacagtcggcttcaccgattccagaaagattagccacagctgcacccaccctttccatcatgacccccccgtgattggaagggagaagttccatagcagacctcaacacggaaaggaactcccttgctccacctatgtgcaaagcaatgccgccaccgccgaagagatcgaggtgcatatgccccccgacacccccgacagaacactgctgagccaacaatccggcaacgtgaagatcacagtcaacagccagaccgtgaggtacaagtgcaactgcggaggctccaatgagggcctcattacaaccgacaaggtcatcaacaactgcaaggtcgaccagtgccacgctgctgtcaccaaccataagaaatggcagtataacagccctctggtgcctagaaatgccgagctgggcgatagaaagggcaagatccatattcccttccctctggccaacgtcacatgtatggtgcccaaggccagaaaccctacagtcacctacggcaaaaatcaagtgatcatgctgctctaccccgatcatcctacactgctgtcctatagaagcatgggagaagagcccaattaccaagaggagtgggtgacccacaaaaaggaggtcgtcctcaccgtgcctacagagggcctcgaggtgacatggggcaataacgagccctacaagtactggccccagctgtccgctaacggaaccgcccacggccacccccatgagattattctgtactattacgagctgtaccccaccatgaccgtggtggtcgtgtccgtcgctagctttattctgctgagcatggtgggaatggccgtcggaatgtgcatgtgtgctagaaggaggtgcattacaccctatgaactgacacccggagctaccgtcccctttctgctgtctctgatctgctgtattagaacagccaaagccgccacatatcaagaagccgccgtctacctctggaatgagcagcagcccctcttttggctgcaagctctgattcccctcgccgctctcatcgtgctgtgcaattgtctgagactgctgccttgctgctgcaaaaccctcgcctttctggctgtcatgagcatcggcgctcacaccgtgagcgcttacgagcacgtcaccgtgatccccaacaccgtgggcgtcccctacaagaccctcgtgaatagacccggctacagccctatggtgctggaaatggagctgctgtccgtcacactggaacccacactgtccctcgactacatcacatgcgagtacaagaccgtcatcccttccccttacgtgaagtgctgcggcaccgccgagtgcaaggacaaaaatctccccgactacagctgcaaggtgttcaccggcgtgtatccctttatgtggggaggcgcctattgcttctgcgacgccgagaatacccaactgtccgaggctcatgtcgagaaatccgagagctgcaaaaccgagttcgccagcgcctacagagcccacacagctagcgcttccgctaagctgagagtgctgtaccaaggcaataacatcaccgtgaccgcttatgccaacggagatcatgccgtcacagtgaaggacgccaaattcattgtgggccccatgagctccgcttggacacccttcgataacaagatcgtcgtgtacaagggcgacgtctacaacatggattaccccccttttggagctggcagacccggccaattcggagatattcaatctagaacccccgaaagcaaggatgtgtacgccaacacccaactcgtgctgcaaaggcccgccgccggaacagtccatgtcccctatagccaagctcctagcggcttcaagtactggctgaaagagaggggagcttctctgcagcatacagctcctttcggatgccagatcgccaccaatcccgtgagggccatgaattgtgccgtcggcaacatgcccatttccatcgatatccccgatgccgctttcacaagggtggtggatgccccctctctgacagacatgagctgtgaagtgcccgcttgtacccacagcagcgacttcggcggagtcgccattattaagtacgccgtgagcaaaaagggcaagtgtgccgtgcacagcatgaccaatgccgtgaccattagagaggccgagatcgaagtcgaaggcaattcccagctccaaatcagctttagcaccgccctcgcctccgctgagttcagagtccaagtgtgcagcacacaagtgcactgtgctgctgagtgtcatccccccaaagaccacatcgtcaactaccccgctagccacacaacactgggcgtgcaagacatctccgctacagccatgagctgggtccaaaagatcaccggaggagtgggactcgtcgtcgctgtcgccgctctgattctgatcgtggtgctctgtgtgtccttctctagacattga

SEQ ID NO: 6 - protein, 1248 aa, Alphavirus chikungunya
MEFIPTQTFYNRRYQPRPWTPRPTIQVIRPRPRPQRQAGQLAQLISAVNKLTMRAVPQQKPRKNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLAIPVMCLLANTTFPCSQPPCIPCCYEKEPEETLRMLEDNVMRPGYYQLLQASLTCSPHRQRRSTKDNFNVYKATRPYLAHCPDCGEGHSCHSPVALERIRNEATDGTLKIQVSLQIGIGTDDSHDWTKLRYMDNHIPADAGRAGLFVRTSAPCTITGTMGHFILARCPKGETLTVGFTDSRKISHSCTHPFHHDPPVIGREKFHSRPQHGKELPCSTYVQSNAATAEEIEVHMPPDTPDRTLLSQQSGNVKITVNSQTVRYKCNCGGSNEGLITTDKVINNCKVDQCHAAVTNHKKWQYNSPLVPRNAELGDRKGKIHIPFPLANVTCMVPKARNPTVTYGKNQVIMLLYPDHPTLLSYRSMGEEPNYQEEWVTHKKEVVLTVPTEGLEVTWGNNEPYKYWPQLSANGTAHGHPHEIILYYYELYPTMTVVVVSVASFILLSMVGMAVGMCMCARRRCITPYELTPGATVPFLLSLICCIRTAKAATYQEAAVYLWNEQQPLFWLQALIPLAALIVLCNCLRLLPCCCKTLAFLAVMSIGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAMNCAVGNMPISIDIPDAAFTRVVDAPSLTDMSCEVPACTHSSDFGGVAIIKYAVSKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 7 - protein, 418 aa, Alphavirus chikungunya
STKDNFNVYKATRPYLAHCPDCGEGHSCHSPVALERIRNEATDGTLKIQVSLQIGIGTDDSHDWTKLRYMDNHIPADAGRAGLFVRTSAPCTITGTMGHFILARCPKGETLTVGFTDSRKISHSCTHPFHHDPPVIGREKFHSRPQHGKELPCSTYVQSNAATAEEIEVHMPPDTPDRTLLSQQSGNVKITVNSQTVRYKCNCGGSNEGLITTDKVINNCKVDQCHAAVTNHKKWQYNSPLVPRNAELGDRKGKIHIPFPLANVTCMVPKARNPTVTYGKNQVIMLLYPDHPTLLSYRSMGEEPNYQEEWVTHKKEVVLTVPTEGLEVTWGNNEPYKYWPQLSANGTAHGHPHEIILYYYELYPTMTVVVVSVASFILLSMVGMAVGMCMCARRRCITPYELTPGATVPFLLSLICCI

SEQ ID NO: 8 - protein, 505 aa, Alphavirus chikungunya
RTAKAATYQEAAVYLWNEQQPLFWLQALIPLAALIVLCNCLRLLPCCCKTLAFLAVMSIGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAMNCAVGNMPISIDIPDAAFTRVVDAPSLTDMSCEVPACTHSSDFGGVAIIKYAVSKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 9 - DNA, 48 bp, Vesicular stomatitis virus
  source 1..48: mol_type = genomic DNA
atgaagtgccttttgtacttagcctttttattcattggggtgaattgc

SEQ ID NO: 10 - protein, 16 aa, Vesicular stomatitis virus
MKCLLYLAFLFIGVNC

SEQ ID NO: 11 - DNA, 195 bp, Vesicular stomatitis virus
  source 1..195: mol_type = genomic DNA
aatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaaggtctga

SEQ ID NO: 12 - protein, 64 aa, Vesicular stomatitis virus
NPIELVEGWFSSWKSSIASFFFIIGLIIGLFLVLRVGIHLCIKLKHTKKRQIYTDIEMNRLGKV

SEQ ID NO: 13 - protein, 7 aa, synthetic construct
PKKKRKV

SEQ ID NO: 14 - protein, 10 aa, synthetic construct
PKKKRKVEAS

SEQ ID NO: 15 - protein, 16 aa, synthetic construct
KRPAATKKAGQAKKKK

SEQ ID NO: 16 - protein, 9 aa, synthetic construct
PAAKRVKLD

SEQ ID NO: 17 - protein, 11 aa, synthetic construct
RQRRNELKRSP

SEQ ID NO: 18 - protein, 38 aa, synthetic construct
NQSSNFGPMKGGNFGGRSSGPYGGGGQYFAKPRNQGGY

SEQ ID NO: 19 - protein, 42 aa, synthetic construct
RMRIZFKNKGKDTAELRRRRVEVSVELRKAKKDEQILKRRNV

SEQ ID NO: 20 - protein, 8 aa, synthetic construct
VSRKRPRP

SEQ ID NO: 21 - protein, 8 aa, synthetic construct
PPKKARED

SEQ ID NO: 22 - protein, 8 aa, synthetic construct
PQPKKKPL

SEQ ID NO: 23 - protein, 12 aa, synthetic construct
SALIKKKKKMAP

SEQ ID NO: 24 - protein, 5 aa, synthetic construct
DRLRR

SEQ ID NO: 25 - protein, 7 aa, synthetic construct
PKQKKRK

SEQ ID NO: 26 - protein, 10 aa, synthetic construct
RKLKKKIKKL

SEQ ID NO: 27 - protein, 10 aa, synthetic construct
REKKKFLKRR

SEQ ID NO: 28 - protein, 20 aa, synthetic construct
KRKGDEVDGVDEVAKKKSKK

SEQ ID NO: 29 - protein, 17 aa, synthetic construct
RKCLQAGMNLEARKTKK

SEQ ID NO: 30 - protein, 401 aa, synthetic construct
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 31 - protein, 432 aa, synthetic construct
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 32 - protein, 461 aa, synthetic construct
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 33 - protein, 439 aa, synthetic construct
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 34 - protein, 419 aa, synthetic construct
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 35 - protein, 1409 aa, synthetic construct
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 36 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 37 - protein, 1365 aa, synthetic construct
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 39 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 40 - protein, 5 aa, synthetic construct
VITYF

SEQ ID NO: 41 - protein, 5 aa, synthetic construct
VVVVV

SEQ ID NO: 42 - protein, 7 aa, synthetic construct
KPWWPRR

SEQ ID NO: 43 - protein, 9 aa, synthetic construct
NIVNVSLVK

SEQ ID NO: 44 - protein, 5 aa, synthetic construct
LPFFD

SEQ ID NO: 45 - protein, 4 aa, synthetic construct
KLVF

SEQ ID NO: 46 - protein, 7 aa, synthetic construct
LVEALYL

SEQ ID NO: 47 - protein, 6 aa, synthetic construct
RGFFYT

SEQ ID NO: 48 - protein, 9 aa, synthetic construct
GGSGGSGGS

SEQ ID NO: 49 - protein, 7 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
PGKLVYA

SEQ ID NO: 50 - protein, 7 aa, unidentified
MYENDLL

(19) United States
(12) Patent Application Publication
(10) Pub. No.: US 2025/0057984 A1
(43) Pub. Date: Feb. 20, 2025

(54) CELL-TYPE SPECIFIC MEMBRANE FUSION PROTEINS

(71) Applicants: The Broad Institute, Inc., Cambridge, MA (US); Massachusetts Institute of Technology, Cambridge, MA (US)

(72) Inventors: Feng ZHANG, Cambridge, MA (US); Daniel STREBINGER, Cambridge, MA (US)

(73) Assignees: The Broad Institute, Inc., Cambridge, MA (US); Massachusetts Institute of Technology, Cambridge, MA (US)

(21) Appl. No.: 18/802,337

(22) Filed: Aug. 13, 2024

Related U.S. Application Data
(63) Continuation of application No. PCT/US2022/052871, filed on Dec. 14, 2022.
(60) Provisional application No. 63/310,324, filed on Feb. 15, 2022.
(60) Provisional application No. 63/335,195, filed on Apr. 26, 2022.

(51) Int. Cl. A61K 48/00; C07K 14/005; C12N 15/86

(52) U.S. Cl. CPC A61K 48/0058; A61K 48/0091; C07K 14/005; C12N 15/86; C12N 2740/15023; C12N 2740/15043; C12N 2740/15045; C12N 2740/15052; C12N 2760/20222; C12N 2770/36122

(57) ABSTRACT

This disclosure is directed to a targeted delivery vehicle that can deliver a cargo to a cell of interest. The targeted delivery vehicle has a fusogen and a targeting domain which are embedded in a lipid bilayer membrane that forms a vesicle, and a cargo within the vesicle. The disclosure is also directed to methods for targeted delivery of cargo using the targeted delivery vehicle described herein.

### CROSS REFERENCE TO RELATED APPLICATION

This application is a continuation of International Application No. PCT/US2022/052871, filed Dec. 14, 2022, which claims the benefit under 35 U.S.C. § 119 (e) of U.S. Provisional Application No. 63/310,324, filed Feb. 15, 2022, and U.S. Provisional Application No. 63/335,195, filed Apr. 26, 2022, the entire contents of which are incorporated herein by reference.

### STATEMENT AS TO FEDERALLY SPONSORED RESEARCH

This invention was made with government support under Grant Nos. HL141201 and HG009761 awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 13, 2024, is named 114203-6135_SL.xml and is 63,564 bytes in size.

### BACKGROUND

The present disclosure relates generally to the field of targeted delivery vehicles. Targeted delivery vehicles are particularly important and useful for gene therapy applications. Current in vivo (e.g., AAV vector, LNP) and ex vivo (e.g., lentiviral vector, electroporation) delivery systems suffer from a variety of drawbacks including improper immune response (inflammatory response, complement inactivation of virus vectors, neutralizing antibodies against vector, innate immune responses of cells against virus or its cargo, pre-existing immunity), off-target effects (e.g., delivery to non-target tissue, manipulation of non-targeted healthy cells), systemic toxicity (e.g., liver toxicity) or insertional mutagenesis that may lead to cancerogenesis or genomic instability. Therefore, there is a need for new generation of modular, specific and versatile targeted delivery vehicles.

Membrane fusion is energetically unfavorable. Fusogens are the proteins that act on the membranes to overcome the forces preventing spontaneous membrane fusion and ensure fusion occurs in a controlled and regulated manner. The first fusogens identified were the viral fusogens. Their existence is immediately apparent in enveloped viruses, such as influenza, HIV, hepatitis, dengue and Zika, which have transmembrane glycoproteins on their surface that are responsible for the attachment and fusion of the viral and host membranes.

### SUMMARY OF THE INVENTION

An aspect of this disclosure is directed to a targeted delivery vehicle comprising: a lipid bilayer membrane, wherein the lipid bilayer membrane forms a vesicle; a fusogen embedded in the lipid bilayer membrane; a targeting moiety embedded in the lipid bilayer membrane, wherein the targeting moiety is separate and different from the fusogen; and a cargo within the vesicle.

In some embodiments, the fusogen is an envelope protein from a virus. In some embodiments, the envelope protein is modified to not have a targeting function.

In some embodiments, the virus is selected from the group consisting of genera Arenaviridae, Filoviridae, Orthomyxoviridae, Rhabdoviridae, Togaviridae, Matonaviridae, Hantaviridae, Bunyaviridae, Retroviridae, Coronaviridae, Bornaviridae and Orthomyxoviridae. In some embodiments, the virus is selected from the group consisting of Pichinde virus, Ebola virus, Dhori virus, Duvenhage lyssavirus, European bat 1 lyssavirus, Isfahan virus, Mokola virus, Rabies virus, Chikungunya virus, Eastern equine encephalitis virus, O'nyong'nyong virus, Rubella virus, Hantaan orthohantavirus, Dugbe virus, La Crosse virus, Influenza A virus, Quaranfil virus, Lassa mammarenavirus, Lymphocytic Choriomeningitis virus, Mammalian Bornavirus 1, Marburg virus, Feline immunodeficiency virus, Rabies virus, Arizona vesiculovirus, Eastern equine encephalitis virus, Semliki Forest virus, Hantaan orthohantavirus, Indiana vesiculovirus, Severe acute respiratory syndrome coronavirus, Severe acute respiratory syndrome coronavirus 2, Influenza A virus, Baboon endogenous virus, Vesicular Stomatitis Virus and Sindbis virus.

In some embodiments, the fusogen is a pH-dependent fusogen. In some embodiments, the pH-dependent fusogen is selected from the group consisting of Sindbis Virus E2 protein, Vesicular Stomatitis Virus G protein, Cocal Virus G protein, and Chikungunya Virus E2 protein.

In some embodiments, the fusogen is the Vesicular Stomatitis Virus G (VSV-G) protein, and wherein the VSV-G protein comprises at least one nonconservative point mutation at a position selected from H8, K47, Y209, and R354. In some embodiments, the VSV-G protein comprises at least one mutation selected from H8A, K47Q, Y209A, and R354Q.

In some embodiments, the fusogen is the Cocal Virus G protein, and wherein the Cocal Virus G protein comprises at least one nonconservative point mutation at a position selected from the group consisting of Q25, K64, Y226, and R371. In some embodiments, the Cocal Virus G protein comprises at least one mutation selected from Q25A, K64Q, Y226A, and R371Q.

In some embodiments, the fusogen is the Chikungunya Virus E2, and wherein the Chikungunya Virus E2 protein comprises at least one nonconservative point mutation at a position selected from the group consisting of W64, D71, T116, 1121, 1190, Y199, and I217. In some embodiments, the fusogen is the Chikungunya Virus E2, wherein the Chikungunya Virus E2 protein comprises at least one mutation selected from the group consisting of D71A, 1121A, 1190A, Y199A, and I217A.

In some embodiments, the fusogen comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

In some embodiments, the targeting moiety comprises a binding domain specific for a target cell of interest. In some embodiments, the binding domain comprises a receptor, an antibody, or an antigen-binding fragment. In some embodiments, the antibody fragment is selected from the group consisting of a Fab, a Fab′, a F(ab′)₂, an Fd, an Fv, a domain antibody, a complementarity determining region (CDR), a single chain variable fragment antibody (scFv), a maxibody, a minibody, an intrabody, a diabody, a triabody, a tetrabody, a v-NAR and a bis-scFv.

In some embodiments, the targeting moiety comprises a tag, and wherein the binding domain is attached to the targeting domain through the tag. In some embodiments, the tag is selected from the group consisting of a SNAP tag, a biotin tag, a monomeric streptavidin, a monomeric streptavidin 2, an intein, a SunTag, an Isopeptag, a SpyTag, a SpyCatcher tag, a SnoopTag, a SnoopTagJr, a SnoopCatcher tag, a DogTag, a DogCatcher tag, a Gluthatione-S-transferase tag, a CLIP tag, a Protein A tag, a Protein G tag, a Protein AG tag, a GFP tag, an HA tag, a FLAG tag and a HiBiT-tag.

In some embodiments, the targeting moiety comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

In some embodiments, the target cell of interest is a mammalian cell. In some embodiments, the target cell of interest is a cancer cell. In some embodiments, the targeted delivery vehicle delivers the cargo to a B cell, a CD4+ T cell, a CD8+ T cell, a lung cell, a colorectal cell, a hematopoietic stem cell, a muscle cell, a cardiac cell, a hepatocyte, a monocyte, a macrophage or a neuronal cell.

In some embodiments, the cargo comprises a deoxyribonucleic acid (DNA), a ribonucleic acid (RNA), a protein, a ribonucleoprotein (RNP) or a combination thereof. In some embodiments, the cargo comprises an expression vector, a gene editing tool, or a gene silencing tool. In some embodiments, the cargo comprises Cre mRNA or Cas9-RNP.

In some embodiments, the targeted delivery vehicle is a pseudotyped lentiviral vector, a selective endogenous encapsidation for cellular delivery system (SEND), a nanoblade, an engineered virus-like particle (eVLP), or a gesicle.

Another aspect of the disclosure is directed to a method for targeted delivery of a cargo comprising administering a targeted delivery vehicle to a subject in need of the cargo, wherein the targeted delivery vehicle comprises: a lipid bilayer membrane, wherein the lipid bilayer membrane forms a vesicle; a fusogen embedded in the lipid bilayer membrane; a targeting moiety embedded in the lipid bilayer membrane, wherein the targeting moiety is separate and different from the fusogen; and a cargo within the vesicle.

In some embodiments, the fusogen is an envelope protein from a virus. In some embodiments, the envelope protein is modified to not have a targeting function.

In some embodiments, the virus is selected from the group consisting of genera Arenaviridae, Filoviridae, Orthomyxoviridae, Rhabdoviridae, Togaviridae, Matonaviridae, Hantaviridae, Bunyaviridae, Retroviridae, Coronaviridae, Bornaviridae and Orthomyxoviridae. In some embodiments, the virus is selected from the group consisting of Pichinde virus, Ebola virus, Dhori virus, Duvenhage lyssavirus, European bat 1 lyssavirus, Isfahan virus, Mokola virus, Rabies virus, Chikungunya virus, Eastern equine encephalitis virus, O'nyong'nyong virus, Rubella virus, Hantaan orthohantavirus, Dugbe virus, La Crosse virus, Influenza A virus, Quaranfil virus, Lassa mammarenavirus, Lymphocytic Choriomeningitis virus, Mammalian Bornavirus 1, Marburg virus, Feline immunodeficiency virus, Rabies virus, Arizona vesiculovirus, Eastern equine encephalitis virus, Semliki Forest virus, Hantaan orthohantavirus, Indiana vesiculovirus, Severe acute respiratory syndrome coronavirus, Severe acute respiratory syndrome coronavirus 2, Influenza A virus, Baboon endogenous virus, Vesicular Stomatitis Virus and Sindbis virus.

In some embodiments, the fusogen is a pH-dependent fusogen. In some embodiments, the pH-dependent fusogen is selected from the group consisting of Sindbis Virus E2 protein, Vesicular Stomatitis Virus G protein, Cocal Virus G protein, and Chikungunya Virus E2 protein.

In some embodiments, the fusogen is the Vesicular Stomatitis Virus G (VSV-G) protein, and wherein the VSV-G protein comprises at least one nonconservative point mutation at a position selected from H8, K47, Y209, and R354. In some embodiments, the VSV-G protein comprises at least one mutation selected from H8A, K47Q, Y209A, and R354Q.

In some embodiments, the fusogen is the Cocal Virus G protein, and wherein the Cocal Virus G protein comprises at least one nonconservative point mutation at a position selected from the group consisting of Q25, K64, Y226, and R371. In some embodiments, the Cocal Virus G protein comprises at least one mutation selected from Q25A, K64Q, Y226A, and R371Q.

In some embodiments, the fusogen is the Chikungunya Virus E2, and wherein the Chikungunya Virus E2 protein comprises at least one nonconservative point mutation at a position selected from the group consisting of W64, D71, T116, 1121, 1190, Y199, and I217. In some embodiments, the fusogen is the Chikungunya Virus E2, wherein the Chikungunya Virus E2 protein comprises at least one mutation selected from the group consisting of D71A, 1121A, 1190A, Y199A, and I217A.

In some embodiments, the fusogen comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

In some embodiments, the targeting moiety comprises a binding domain specific for a target cell of interest. In some embodiments, the binding domain comprises a receptor, an antibody, or an antigen-binding fragment. In some embodiments, the antibody fragment is selected from the group consisting of a Fab, a Fab′, a F(ab′)₂, an Fd, an Fv, a domain antibody, a complementarity determining region (CDR), a single chain variable fragment antibody (scFv), a maxibody, a minibody, an intrabody, a diabody, a triabody, a tetrabody, a v-NAR and a bis-scFv.

In some embodiments, the targeting moiety comprises a tag, and wherein the binding domain is attached to the targeting domain through the tag. In some embodiments, the tag is selected from the group consisting of a SNAP tag, a biotin tag, an Isopeptag, a SpyTag, a SpyCatcher tag, a SnoopTag, a SnoopTagJr, a SnoopCatcher tag, a DogTag, a DogCatcher tag, a Gluthatione-S-transferase tag, a CLIP tag, a Protein A tag, a Protein G tag, a Protein AG tag, a GFP tag, an HA tag, a FLAG tag and a HiBiT-tag.

In some embodiments, the targeting moiety comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

In some embodiments, the target cell of interest is a mammalian cell. In some embodiments, the target cell of interest is a cancer cell. In some embodiments, the targeted delivery vehicle delivers the cargo to a B cell, a CD4+ T cell, a CD8+ T cell, a lung cell, a colorectal cell, a hematopoietic stem cell, a muscle cell, a cardiac cell, a hepatocyte, a monocyte, a macrophage or a neuronal cell.

In some embodiments, the cargo comprises a deoxyribonucleic acid (DNA), a ribonucleic acid (RNA), a protein, a ribonucleoprotein (RNP) or a combination thereof. In some embodiments, the cargo comprises an expression vector, a gene-editing tool, or a gene-silencing tool. In some embodiments, the cargo comprises Cre mRNA or Cas9-RNP.

In some embodiments, the targeted delivery vehicle is a pseudotyped lentiviral vector, a selective endogenous encapsidation for cellular delivery system (SEND), a nanoblade, an engineered virus-like particle (eVLP), or a gesicle.

In some embodiments, the targeted delivery vehicle is administered locally or systemically.

### BRIEF DESCRIPTION OF THE DRAWINGS

FIGS. 1A-1C. Pseudotyped lentiviral vectors (A) Exemplary constructs to make a library of pseudotyped lentiviruses. (B) Schematic of pseudotyped lentiviruses. (C) Approach for screening pseudotyped lentiviruses.

FIGS. 2A-2N. (A) 3 dimensional structure of Sindbis virus E2 protein. Magenta: wild type E2 protein; Red: Protein A fused E2 protein. (B) Protein A fused Sindbis virus E2 protein can target Ace2 expressing cells in the presence of anti-Ace2 antibodies. Briefly, 5000 A549+Ace2 cells were incubated with 10 μl virus with or without lul aAce2 antibody. The results were analyzed by flow Cytometry after 7 days. (C) 3 dimensional structure of Sindbis virus E2 protein showing that a SNAP tag can be used in the place of protein A. (D) Depiction of SNAP-tag (click chemistry)-mediated retargeting of a viral envelope. (E)-(F) SNAP-tag can successfully and specifically target viral envelope proteins to Ace2+ cells in the presence of an Ace2 antibody. Lower targeting molecule/fusogen ratio increases infectivity. (G) Schematic of separating targeting and fusion functions. (H)-(I) High levels of targeting moieties result in lower transduction. (J)-(K) Expression of the helper Envelope from a different promoter increases production of viral particles. (L)-(N) Antibody-based retargeting of Sindbis Envelope allows specific transduction of target cells.

FIGS. 3A-3O. (A) Re-targeting works with Chikungunya virus envelope protein (CHIKV) using protein AG (pAG). (B) Pseudotyped viruses that displayed cocal viral envelope protein (COCV) fused to protein AG (COCV+pAG) specifically and efficiently targeted HEK293FT cells in the presence of MHCI antibodies. (C)-(D) Inherent tropisms of viral envelope proteins. (E)-(F) Vesicular Stomatitis Virus Envelope protein (VSV-G) mutations decrease infectivity. (G)-(H) Double mutants of VSV-G decrease infection even further. (I) Adherent cell line panel. Indicated amounts of VSVg K47Q, R354Q double mutant tested on seven adherent cell lines. Some cell lines show high basal transduction (A172, HUH7). All cell lines express ClassI (confirmed by flow cytometry). Presence of aClassI antibody boosts infection rates up to 30-fold (e.g., on NCIH-358). (J) Titration of CD3 antibody amount on VSVG K47Q+R354Q double mutant. 50000 Jurkat T cells were infected with the indicated amounts of concentrated virus (y-axis) that was pre-incubated with the indicated amounts of antibody (x-axis). In the absence of antibody, there is very low transduction (white squares). (K) Jurkat-Surf-GFP cells can be transduced with aGFP targeted virus: VSV-G K47Q-R354Q double mutant virus were targeted to Jurkat+surfGFP cells (Jurkat cells expressing GFP on their surface) with protein AG (pAG) and anti-GFP antibodies. Briefly, Jurkat+surfGFP were transduced with indicated amounts of concentrated virus (1000×) (FIG. 3K). Cells were stained with aGFP antibody (homebrew) and subsequently incubated with indicated virus amounts (FIG. 3K). The cells were analyzed by flow cytometry after 5 days. (L) Chikungunya envelope engineering. Infectivity decreases are the most pronounced for conserved residues. (M)-(N) Chikungunya virus envelope mutations can decrease infectivity. (O) HEK293FT cells expressing GFP on their surface (HEK293FT+Surf-GFP) transduced with aGFP targeted viruses. HEK 293T+surfGFP cels were transduced with indicated amounts of concentrated virus (100×). All conditions were in the presence of a commercial aGFP antibody. Cells were analyzed for high and low GFP on target cells.

FIGS. 4A-4G. (A) Different alternatives exist for engineering targeting and fusion functions. (B) scFvs can be used as targeting molecules. Viruses containing CHIKV-E1E2 with anti-HA scFv instead of protein AG were produced. HEK293FT+Surf-HA cells were target cells and HEK293FT cells were nontarget cells. Anti-HA scFv successfully targeted the virus to HA expressing HEK293FT cells. (C) Directed Cocal envelope works with SEND. 5000 A549+Ace2+CreReporter cells were incubated with 30 μl virus+1 μl aAce2 antibody. Cells were analyzed by Flow Cytometry after 3 days. (D) Classes of fusogens. (E) GP64 can be retargeted and withstands freeze-thaw. Indicated amounts of unconcentrated virus were used on 10000 HEK293FT cells. Increased infection in presence of antibody (˜5-fold increase) was observed. (F) Scheme of cargo delivery using pH-dependent fusogens. (G) Phoenix (ERV-K). ERV-K transcripts have been detected in multiple tissues, including breast, colon, kidney, placenta, prostate, testis, trachea, and thymus. Indicated amounts of 100× concentrated virus (in μl) on 10000 HEK293FT cells. Analyzed by flow cytometry after 4 days. Increased infection in presence of antibody (˜6-fold).

FIGS. 5A-5D: Development of a modular delivery system-Delivery to Intended REcipient Cells Through Envelope Design (DIRECTED) (A) Co-expression of protein AG (pAG) together with a viral fusogen (VSIV-G) allows to expand the intrinsic tropism of VSIV-G in the presence of an antibody targeting a surface receptor expressed on target cells (HEK293FT). (B) Blocking of the intrinsic receptor binding capability of VSIV-G by co-incubation with a competitor (dimeric CR2 domain derived from human LDL-R) makes transduction completely dependent on the presence of the antibody. (C) Genetic abolishment of intrinsic tropism of VSIV-G identifies VSIV-G double mutants which produce at ˜50% efficiency of the wildtype version, but show high attenuation of infection. (D) Combining a VSIV-G double mutant (K47Q, R354Q) with pAG results in a highly modular delivery system where tropism can be determined by an antibody targeting cell surface receptors on target cells.

FIGS. 6A-6E: Specificity of DIRECTED and expansion of targeting strategies. (A)-(B) The antibody amount determines the efficiency of cargo delivery, but is robust over a 4-fold range. Jurkat T cells were co-incubated with different amounts of DIRECTED-Lentiviral vectors, delivering an H2B-mCherry transgene, and varying amounts of αCD3 antibody. (C) Co-cultures of Jurkat T cells (CD3+) and K562 cells (HLA-A2+) at different ratios are challenged with DIRECTED-Lentiviral vectors in the presence of an αCD3 antibody, an α-HLA-A2 antibody, or in the absence of antibody and the amount of cells expressing mCherry is determined by Flow cytometry 4 days later. DIRECTED allows targeting of surface marker expressing cells with high efficiency and shows low background in the absence of antibody. (D) Expression of a membrane-bound single chain variable fragment (scFv) against HA on DIRECTED-Lentiviral vectors enables targeting of and transgene delivery (H2B-mCherry) to cells that express a synthetic Surface HA (SurfHA) receptor. (E) A SNAP tag, which interacts with Benzylguanine residues by forming a covalent bond, can be expressed on the surface of enveloped viral vectors and enables targeting of Ace2+ cells after co-incubation with an Anti-Ace2-Benzylguanine antibody.

FIGS. 7A-7H: Additional envelopes can be used with DIRECTED. (A)-(B) A sequence-based analysis reveals multiple candidates, including Vesicular Stomatitis Indiana virus G and Cocal virus G. (C) Cocal virus G can be effectively redirected in the presence of protein AG (pAG). (D) Screening of a library of ˜100 viral fusogens identifies proteins from multiple viral families that can be harnessed for DIRECTED. The families are Filoviridae (FiV), Orthomyxoviridae (OrmyV), Rhabodviridae and Togaviridae. All of these families have been reported to use a pH-dependent uptake mechanism. (E)-(F) A sequence-based homology search for Orthomyxoviral envelopes reveals multiple candidates, including the surface protein from Quaranfil quaranjavirus (QRFV) and Dhori thogotovirus (DHOV), which were part of the initial library, as well as baculoviral GP64. (G) Baculoviral GP64 can be effectively redirected in the presence of protein AG (pAG). (H) Exemplary embodiments of the fusogens and targeting moieties.

FIGS. 8A-8B: DIRECTED can be combined with tools to deliver RNPs and mRNA. (A) DIRECTED-eVLPs allow the specific knockout of B2M in Jurkat cells only upon targeting via an Anti-CD3 antibody. Data represents surface protein expression as determined by Flow cytometry. (B) DIRECTED-SEND can be used to deliver Cas9 mRNA and sgRNAs to Jurkat T cells in the presence of Anti-CD3 or Anti-CD5 antibodies, but not in the absence of a targeting antibody. Data represents the surface protein expression as determined by Flow cytometry.

FIG. 9 shows a protein level readout for H2B-mCherry delivered using a lentiviral vector coexpressing VSV-G (K47Q, R354Q) and a membrane-bound SNAP tag (SNAP-TM) analyzed 3 days after transduction of primary mouse splenocytes. The viral vector preparation was either co-incubated with αCD5-Benzylguanine (against mouse) or with no antibody.

FIGS. 10A-10E. (A)-(C) Retro-orbital injection of 3 mice each with VSIV-G, VSIV-G (K47Q, R354Q)+pAG (dmp), or dmp+αMHC-ClassI, at ˜1E11 lentiviral particles per mouse, with H2B-mCherry-P2A-NanoLuc as the transgene. Compared to lentiviral vectors with VSIV-G envelop, lentiviral vectors with dmp and dmp+αMHC-ClassI envelops show 1.8-fold and 4.4-fold reduction in mCherry signals in liver cells, respectively, thereby demonstrating liver de-targeting. (D)-(E) Retro-orbital injection of 3 mice each with VSIV-G, VSIV-G (K47Q, R354Q)+pAG (dmp), or dmp+αMHC-ClassI, at ˜1E11 lentiviral particles per mouse, with H₂B-mCherry-P2A-NanoLuc as the transgene. Compared to lentiviral vectors with VSIV-G envelop, lentiviral vectors with dmp and dmp+αMHC-ClassI envelops show 1.7-fold and 2.9-fold reduction in mCherry signals in spleen B cells, respectively, thereby demonstrating spleen de-targeting.

FIG. 11. Exemplary protocols for optional HSC mobilization.

FIGS. 12A-12D. Mixing of target and non-target cells. (A) Surface-HA HEK293FT cells (target cells) were mixed with HEK293FT cells (non-target cells). Two preparation of VSV-G K47Q,R354Q+protein AG (pAG) virus were produced and titrated using RT-qPCR for viral genomes (VGs). Incubated with two preparations of VSV-G K47Q,R354Q+protein AG (pAG) virus in presence or absence of targeting antibody at different multiplicities of infection (MOIs; 500, 750, 1000 VGs/cell). ˜25% of cells were Surface-HA+ (target cells) in all conditions as determined by flow cytometry after staining with aHA-PB450. (B) Overall infection of cells was determined by Flow cytometry (detecting the H2B-mCherry transgene that was delivered). Presence of antibody shows a boost of cells that get infected over no antibody pAG2 preparation shows higher transduction efficiency overall. (C)-(D) Analysis of the percentage of successfully transduced target cells in presence of aHA antibody reveals high percentage of target cells transduced. In the absence of an antibody the frequency of target cells is ˜25%, which would be expected due to the frequency with which target cells are represented in the population. Of the ˜1% of cells that were transduced at MOI 1000 in the absence of antibody 70-75% were non-target cells and 25-30% were Surface-HA+ (target cells).

FIGS. 13A-13B. Comparison of SNAP and proteinAG (pAG) strategy for targeting of different receptors expressed on Surface-HA+Jurkat cells. (A) SNAP shows high transduction efficiency in the presence of αHA-BG, αCD5-BG, αCD46-BG, and αCD3-BG. As expected, αHA without benzylguanine does not increase transduction. Similarly, absence of any antibody does not result in successful infection of Surface-HA+Jurkat cells. (B) pAG allows efficient transduction of Surface-HA+Jurkat cells with αHA, and αCD3. However, αCD5 and αCD46 do not allow efficient infection of Surface-HA+Jurkat cells. This may be caused by different turnover rates of the target receptors. While SNAP results in covalent immobilization of antibody-BG substrates on virions, the pAG strategy relies on protein-protein interactions, which are intrinsically more transient.

FIGS. 14A-14B. Targeting of CD117 (c-Kit) on Kasumi-1 cells. (A) Viral particles were incubated with antibody-BG for 15 minutes at room temperature, before excess antibody-BG was removed by ultrafiltration (100 kDa cutoff) by washing for 3 times with an excess of PBS. (B) CD117 (c-Kit) is a receptor that is highly expressed on hematopoietic stem cells (HSCs), therefore representing an attractive target for gene delivery to HSCs. Using CD117+Kasumi-1 cells we could show that co-incubation of VSV-G mutant K47Q, R354Q+SNAP particles with αCD117-BG result in efficient transduction of these cells, to levels similar to VSV-G. αCD20-BG and the absence of antibody show no successful transduction as evaluated by Flow cytometry for the H2B-mCherry transgene.

FIG. 15. Overview of virus harvest and purification.

FIG. 16. Analysis of transduced cells in vivo in liver Kupffer cells. In animals injected with VSV-G, an increase in macrophages was observed. Overall a similar amount of cells were transduced. Percentage of Kupffer cells of all tdTomato+ cells was ˜50-60%. All variants were observed to cause infection of Kupffer cells with high efficiency. However, only VSV-G causes an increase in Kupffer cells, which could be indicative of an immune response. Most of the virus seems to be taken up by macrophages.

FIGS. 17A-17B. Analysis of transduced cells in vivo-Spleen. (A) Overall transduction. (B) Normalized transduction. Overall similar transduction efficiencies were observed in all conditions. Majority of transduced cells are CD20+ (B cells)

FIG. 18. Analysis of transduced cells in vivo-Spleen CD20+ cells. CD20 relative cell abundance was reduced in VSV-G condition. Overall a similar amount of cells was transduced. Around 50-70% of transduced cells are CD20+. CD20+ cells are the most efficiently transduced in all conditions. Decrease in CD20+ cells in spleens could indicate inflammation in VSV-G condition.

### DETAILED DESCRIPTION

### Definitions

As used herein, the term “fusogen” refers to an agent or molecule (e.g. a protein) that creates an interaction between two membrane-enclosed lumens. In some embodiments, fusogen promotes membrane fusion. In another embodiment, fusogen creates a connection, e.g., a pore, between two lumens (e.g., the lumen of a retroviral vector and the cytoplasm of a target cell).

In some embodiments, the fusogen is a re-targeted fusogen. As used herein, a “re-targeted fusogen” refers to a fusogen that comprises a targeting moiety having a sequence that is not part of the naturally-occurring form of the fusogen. In some embodiments, the fusogen comprises a different targeting moiety relative to the targeting moiety in the naturally-occurring form of the fusogen. In some embodiments, the naturally-occurring form of the fusogen lacks a targeting domain, and the re-targeted fusogen comprises a targeting moiety that is absent from the naturally-occurring form of the fusogen. In some embodiments, the fusogen is modified to comprise a targeting moiety. In some embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “gesicle” is a delivery system comprising a microvesicle secreted by a eukaryotic cell overexpressing a viral membrane fusion protein and a protein of interest as described in Mangeot, P E, et al., Molecular Therapy, 19.9 (2011): 1656-1666, and U.S. Pat. No. 9,695,446B2 which are incorporated herein in its entirety.

As used herein, a “selective endogenous encapsidation for cellular delivery system” (“SEND”) is a delivery system that comprises non-naturally occurring self-assembling polypeptides for transferring nucleic acids and/or proteins to a cell as described in Segel, M., et al., Science, 373.6557 (2021): 882-889, and US20200347100A1 which are incorporated herein in its entirety.

As used herein, a “nanoblade” is a delivery system that comprises a virus-derived particle as described in Mangeot, P E., et al., Nature Communications, 10.1 (2019): 1-15, and US20210284697A1 which are incorporated herein in its entirety.

As used herein, the phrase “conservative substitution” refers to substituting an amino acid residue for a different amino acid residue that has similar chemical properties. Exemplary conservative substitutions include the ones listed in the table below.

### Amino Acids and Substitutions

| | Conservative | |
|---|---|---|
| Original Residue | Substitutions | Exemplary Substitutions |
| alanine Ala (A) | Val | Val; Leu; Ile |
| arginine Arg (R) | Lys | Lys; Gln; Asn |
| asparagine Asn (N) | Gln | Gln; His; Asp, Lys; Arg |
| aspartatic Asp (D) | Glu | Glu; Asn |
| cysteine Cys (C) | Ser | Ser; Ala |
| glutamine Gln (Q) | Asn | Asn; Glu |
| glutamic Glu (E) | Asp | Asp; Gln |
| glycine Gly (G) | Ala | Ala |
| histidine His (H) | Arg | Asn; Gln; Lys; Arg |
| isoleucine Ile (I) | Leu | Leu; Val; Met; Ala; Phe; |
| | | Norleucine |
| leucine Leu (L) | Ile | Norleucine; Ile; Val; Met; |
| | | Ala; Phe |
| lysine Lys (K) | Arg | Arg; Gln; Asn |
| methionine Met (M) | Leu | Leu; Phe; Ile |
| phenylalanine Phe (F) | Tyr | Leu; Val; Ile; Ala; Tyr |
| proline Pro (P) | Ala | Ala |
| serine Ser (S) | Thr | Thr |
| threonine Thr (T) | Ser | Ser |
| tryptophan Trp (W) | Tyr | Tyr; Phe |
| tyrosine Tyr (Y) | Phe | Trp; Phe; Thr, Ser |
| valine Val (V) | Leu | Ile; Leu; Met; Phe; Ala; |
| | | Norleucine |

As used herein, the phrase “nonconservative substitution” refers to substituting an amino acid residue for a different amino acid residue that has different chemical properties. The nonconservative substitutions include, but are not limited to aspartic acid (D) replaced with glycine (G); asparagine (N) replaced with lysine (K); or alanine (A) replaced with arginine (R).

Naturally occurring residues in amino acids are divided into groups based on common side-chain properties:
i. Non-polar: Norleucine, Met, Ala, Val, Leu, Ile;
ii. Polar without charge: Cys, Ser, Thr, Asn, Gln;
iii. Acidic (negatively charged): Asp, Glu;
iv. Basic (positively charged): Lys, Arg;
V. Residues that influence chain orientation: Gly, Pro; and
vi. Aromatic: Trp, Tyr, Phe, His.

In some embodiments, non-conservative substitutions are made by exchanging a member of one of these groups (based on common side chain properties) for another class.

### Targeted Delivery Vehicle

An aspect of this disclosure is directed to a targeted delivery vehicle comprising: a lipid bilayer membrane, wherein the lipid bilayer membrane forms a vesicle; a fusogen embedded in the lipid bilayer membrane; a targeting moiety embedded in the lipid bilayer membrane, wherein the targeting moiety is separate and different from the fusogen; and a cargo within the vesicle.

In some embodiments, the fusogen is an envelope protein from a virus. In some embodiments, the envelope protein is modified to not have a targeting function.

In some embodiments, the virus is selected from the group consisting of genera Arenaviridae, Filoviridae, Orthomyxoviridae, Rhabdoviridae, Togaviridae, Matonaviridae, Hantaviridae, Bunyaviridae, Retroviridae, Coronaviridae, Bornaviridae and Orthomyxoviridae. In some embodiments, the virus is selected from the group consisting of Pichinde virus, Ebola virus, Dhori virus, Duvenhage lyssavirus, European bat 1 lyssavirus, Isfahan virus, Mokola virus, Rabies virus, Chikungunya virus, Eastern equine encephalitis virus, O'nyong'nyong virus, Rubella virus, Hantaan orthohantavirus, Dugbe virus, La Crosse virus, Influenza A virus, Quaranfil virus, Lassa mammarenavirus, Lymphocytic Choriomeningitis virus, Mammalian Bornavirus 1, Marburg virus, Feline immunodeficiency virus, Rabies virus, Arizona vesiculovirus, Eastern equine encephalitis virus, Semliki Forest virus, Hantaan orthohantavirus, Indiana vesiculovirus, Severe acute respiratory syndrome coronavirus, Severe acute respiratory syndrome coronavirus 2, Influenza A virus, Baboon endogenous virus, Vesicular Stomatitis Virus and Sindbis virus.

In some embodiments, the fusogen is an endogenized viral envelope protein. In some embodiments, the endogenized viral envelope protein is Syn1, Syn2, SynA, SynB, or ERV-K180.

In some embodiments, the fusogen is a C2-domain containing protein such as Perforin 1, and Synaptotagmin.

In some embodiments, the fusogen is a pH-dependent fusogen (e.g., the fusogen has a pH-dependent uptake mechanism). In some embodiments, the pH-dependent fusogen is selected from the group consisting of Sindbis Virus E2 protein, Vesicular Stomatitis Virus G protein, Cocal Virus G protein, and Chikungunya Virus E2 protein.

In some embodiments, the fusogen is a Vesicular Stomatitis Virus G (VSV-G) protein. In some embodiments, the VSV-G protein comprises a sequence that is at least 80%, 85%, 90%, 95%, 99% or more identical to SEQ ID NO: 2, or the VSV-G protein is encoded by a nucleic acid sequence that is at least 80%, 85%, 90%, 95%, 99% or more identical to SEQ ID NO: 1. In some embodiments, the VSV-G protein comprises at least one nonconservative point mutation at a position selected from H8 (corresponding to H24 of SEQ ID NO: 2), K47 (corresponding to K63 of SEQ ID NO: 2), Y209 (corresponding to Y225 of SEQ ID NO: 2), and R354 (corresponding to R370 of SEQ ID NO: 2). In some embodiments, the VSV-G protein comprises at least one mutation selected from H8A, K47Q, Y209A, and R354Q.

In some embodiments, the fusogen is the Cocal Virus G protein. In some embodiments, the Cocal Virus G protein comprises a sequence that is at least 80%, 85%, 90%, 95%, 99% or more identical to SEQ ID NO: 4, or the Cocal Virus G protein is encoded by a nucleic acid sequence that is at least 80%, 85%, 90%, 95%, 99% or more identical to SEQ ID NO: 3. In some embodiments, the Cocal Virus G protein comprises at least one nonconservative point mutation at a position selected from the group consisting of Q25, K64, Y226, and R371 of SEQ ID NO: 4. In some embodiments, the Cocal Virus G protein comprises at least one mutation selected from Q25A, K64Q, Y226A, and R371Q.

In some embodiments, the fusogen is a Chikungunya Virus E2 protein. In some embodiments, the Chikungunya Virus E2 protein comprises a sequence that is at least 80%, 85%, 90%, 95%, 99% or more identical to SEQ ID NO: 8. In some embodiments, the Chikungunya Virus E2 protein comprises at least one nonconservative point mutation at a position selected from the group consisting of W64, D71, T116, 1121, 1190, Y199, and I217 (according to the amino acid positions shown in SEQ ID NO: 8). In some embodiments, the fusogen is the Chikungunya Virus E2, wherein the Chikungunya Virus E2 protein comprises at least one mutation selected from the group consisting of D71A, 1121A, 1190A, Y199A, and I217A.

In some embodiments, the fusogen comprises a transmembrane domain. In some embodiments, the transmembrane domain is selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

In some embodiments, the targeting moiety comprises a binding domain specific for a target cell of interest. In some embodiments, the binding domain comprises a receptor, an antibody, or an antigen-binding fragment. In some embodiments, the antibody fragment is selected from the group consisting of a Fab, a Fab′, a F(ab′)₂, an Fd, an Fv, a domain antibody, a complementarity determining region (CDR), a single chain variable fragment antibody (scFv), a maxibody, a minibody, an intrabody, a diabody, a triabody, a tetrabody, a variable domain of new antigen receptor (v-NAR) and a bispecific scFv (bis-scFv).

In some embodiments, the targeting moiety comprises a tag, and wherein the binding domain is attached to the targeting domain through the tag. In some embodiments, the tag is selected from the group consisting of a SNAP tag, a biotin tag, an Isopeptag, a SpyTag, a SpyCatcher tag (Spy Tag and SpyCatcher tag are defined in Reddington, Samuel C., and Mark Howarth. Current opinion in chemical biology 29 (2015): 94-99, which is incorporated herein in its entirety), a SnoopTag, a SnoopTagJr, a SnoopCatcher tag (SnoopTag, SnoopTagJr and SnoopCatcher tags are as defined in Hatlem, Daniel, et al., International journal of molecular sciences 20.9 (2019): 2129, which is incorporated herein in its entirety), a DogTag, a DogCatcher tag (DogTag and DogCatcher tags are as defined in Keeble, Anthony H., et al. Cell chemical biology (2021), which is incorporated herein in its entirety), a Gluthatione-S-transferase tag, a CLIP tag (a modified version of SNAP-tag. It is also a self-labeling protein derived from human 06-alkylguanine-DNA-alkyltransferase. Instead of benzylguanine derivatives, CLIP tag is engineered to react with benzylcytosine derivatives), a Protein A tag, a Protein G tag, a Protein AG tag, a GFP tag, an HA tag, a FLAG tag and a HiBiT-tag (as described in Hoare, Bradley L., et al., Pharmacology research & perspectives 7.4 (2019): e00513, which is incorporated herein in its entirety).

In some embodiments, the targeting moiety comprises a secretion signal (e.g., the secretion signal from VSV-G) in addition to a transmembrane domain that allows the targeting moiety to end up on the lipid bilayer surface.

In some embodiments, the targeting moiety comprises a transmembrane domain selected from the group consisting of a Vesicular Stomatitis Virus G C terminal domain (VSVG-CTD), a transmembrane domain of Beta-2 microglobulin (B2M), a transmembrane domain of Human Leukocyte Antigen-A (HLA-A), and a transmembrane domain of platelet derived growth factor receptor beta (PDGFRB-TM).

In some embodiments, the target cell of interest is a mammalian cell. In some embodiments, the target cell of interest is a cancer cell. In some embodiments, the targeted delivery vehicle delivers the cargo to a B cell, a CD4+ T cell, a CD8+ T cell, a lung cell, a colorectal cell, a hematopoietic stem cell, a muscle cell, a cardiac cell, a hepatocyte, a monocyte, a macrophage or a neuronal cell.

In some embodiments, the cargo comprises a deoxyribonucleic acid (DNA), a ribonucleic acid (RNA), a protein, a ribonucleoprotein (RNP) or a combination thereof. In some embodiments, the cargo comprises an expression vector, a gene editing tool, or a gene silencing tool. In some embodiments, the cargo comprises Cre mRNA or Cas9-RNP.

In some embodiments, the targeted delivery vehicle is a pseudotyped lentiviral vector, a selective endogenous encapsidation for cellular delivery system (“SEND”), a nanoblade, an engineered virus-like particle (eVLP), or a gesicle.

In some embodiments, the targeting vehicle comprises a targeting molecule and a separate and different fusogen, wherein the ratio of the amount of targeting molecule to the amount of fusogen is between about 1:1.5 (meaning there are about 1.5 fusogen molecules for every 1 targeting molecule) and about 1:100 (meaning there are about 100 fusogen molecules for every 1 targeting molecule) on the targeting vehicle. In some embodiments, the ratio of targeting molecule and fusogen is about 1:2, about 1:3, about 1:4, about 1:5, about 1:7, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, or about 1:100. As used in this disclosure, the term “about” refers to +10% of a given value.

In some embodiments, the disclosure is directed to modular and specific expansion of tropism of any delivery technology that contains a membrane, which includes not only biologically derived membranes (e.g., retroviral vectors, alpha virus based vectors, baculoviral vectors, extracellular vesicles, and VLPs) but also synthetic membrane containing particles (e.g., liposomes).

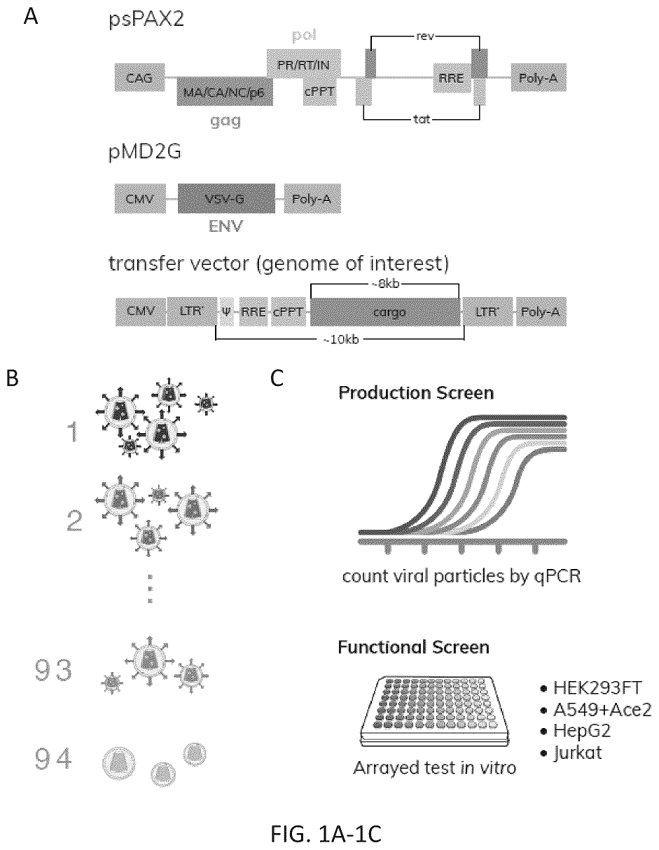

Another aspect of the disclosure relates to a vector system for producing the targeted delivery vehicle, wherein the vector system comprises one or more vectors encoding the fusogen and the targeting moiety (FIG. 1A). Another aspect of the disclosure relates to a host cell comprising or transformed with the vector system (FIG. 1A). Another aspect of the disclosure relates to a host cell for producing the targeted delivery vehicle, wherein the host cell comprising one or more polynucleotides encoding the fusogen and the targeting moiety (FIG. 1A). Another aspect of the disclosure relates to a method for producing the targeted delivery vehicle, comprising expressing one or more polynucleotides encoding the fusogen and the targeting moiety in a host cell in the presence of the cargo (FIG. 1A).

### Pseudotyped Lentiviral Vector

A further aspect of the disclosure is directed to modular and specific expansion of tropism of a lentiviral vector delivery system. In some embodiments, the lentiviral vector system consists of vector particles bearing glycoproteins (GPs) derived from other enveloped viruses. Such particles possess the tropism of the virus from which the GP was derived. In some embodiments, the lentiviral vector system's endogenous GP is replaced with a fusogen (e.g., an engineered VSV-G with decreased or substantially eliminated infectivity) and a targeting moiety (e.g., an antibody or antigen-binding molecule) that is separate and different from the fusogen, as described herein, resulting in expansion of the tropism of the lentiviral vector system. In some embodiments, the fusogen is a pH-dependent fusogen. In some embodiments, the pH-dependent fusogen is selected from the group consisting of Sindbis Virus E2 protein, Vesicular Stomatitis Virus G protein, Cocal Virus G protein, and Chikungunya Virus E2 protein. In some embodiments, the lentiviral vector system with expanded tropism is used to package and deliver an mRNA encoding a CRISPR-Cas protein or a fusion protein thereof (e.g., a base editor or prime editor). In some embodiments, the lentiviral vector system with expanded tropism is used to package and deliver an mRNA encoding a CRISPR-Cas protein or a fusion protein thereof (e.g., a base editor or prime editor), and a gRNA of the CRISPR-Cas protein.

### Send

A further aspect of the disclosure is directed to modular and specific expansion of tropism of a selective endogenous encapsidation for cellular delivery system (“SEND”). In some embodiments, the VSV-G fusogen of the SEND system as described in Segel, M., et al., Science, 373.6557 (2021): 882-889, is replaced with a fusogen (e.g., an engineered VSV-G with decreased or substantially eliminated infectivity) and a targeting moiety (e.g., an antibody or antigen-binding molecule) that is separate and different from the fusogen, as described herein, resulting in expansion of the tropism of the SEND system. In some embodiments, the SEND system with expanded tropism is used to package and deliver an mRNA encoding a CRISPR-Cas protein or a fusion protein thereof (e.g., a base editor or prime editor). In some embodiments, the SEND system with expanded tropism is used to package and deliver an mRNA encoding a CRISPR-Cas protein or a fusion protein thereof (e.g., a base editor or prime editor), and a gRNA of the CRISPR-Cas protein. In some embodiments, the mRNA encoding a CRISPR-Cas protein or a fusion protein thereof is flanked by Peg10 UTR sequences, as described in Segel, M., et al., Science, 373.6557 (2021): 882-889, which is incorporated herein by reference in its entirety.

### Nanoblade

A further aspect of the disclosure is directed to modular and specific expansion of tropism of a nanoblade delivery system. In some embodiments, the VSV-G and BaEVRLess fusogen of the nanoblade system as described in Mangeot et al., Nature Communications, 10.1 (2019): 1-15, is replaced with a fusogen (e.g., an engineered VSV-G with decreased or substantially eliminated infectivity) and a targeting moiety (e.g., an antibody or antigen-binding molecule) that is separate and different from the fusogen, as described herein, resulting in modular and specific expansion of the tropism of the nanoblade system. In some embodiments, the nanoblade system with expanded tropism is used to package and deliver a CRISPR-Cas protein or a fusion protein thereof (e.g., a base editor or prime editor). In some embodiments, the nanoblade system with expanded tropism is used to package and deliver an ribonucleoprotein comprising a CRISPR-Cas protein or a fusion protein thereof (e.g., a base editor or prime editor), and a gRNA of the CRISPR-Cas protein. In some embodiments, the CRISPR-Cas protein or a fusion protein thereof is fused to a gag protein (e.g., ML Vgag) via a cleavable linker, wherein cleavage of the linker in the target cell exposes a nuclear localization signal (NLS) positioned between the linker and the CRISPR-Cas protein or fusion protein, as described in Banskota, et al., Cell, 185 (2021): 1-16, which is incorporated herein by reference in its entirety. In some embodiments, the fusion protein comprises (e.g., from 5′ to 3′) a gag protein (e.g., MLVgag), one or more nuclear export signals (NESs), a cleavable linker, one or more nuclear localization signals (NLSs), and a CRISPR-Cas protein or a fusion protein thereof (e.g., a base editor or prime editor).

### Gesicle

A further aspect of the disclosure is directed to modular and specific expansion of tropism of a gesicle delivery system. In some embodiments, the VSV-G fusogen of the gesicle system as described in Mangeot, P E, et al., Molecular Therapy, 19.9 (2011): 1656-1666, is replaced with a fusogen (e.g., an engineered VSV-G with decreased or substantially eliminated infectivity) and a targeting moiety (e.g., an antibody or antigen-binding molecule) that is separate and different from the fusogen, as described herein, resulting in expansion of the tropism of the gesicle system. In some embodiments, the gesicle system with expanded tropism is used to package and deliver a CRISPR-Cas protein or a fusion protein thereof (e.g., a base editor or prime editor). In some embodiments, the gesicle system with expanded tropism is used to package and deliver a ribonucleoprotein comprising a CRISPR-Cas protein or a fusion protein thereof (e.g., a base editor or prime editor), and a gRNA of the CRISPR-Cas protein. In some embodiments, the CRISPR-Cas protein or a fusion protein thereof is fused to a first dimerizable domain capable of dimerization or heterodimerization with a second dimerizable domain fused to a membrane protein, wherein presence of a ligand facilitates said dimerization and enriches the CRISPR-Cas protein or a fusion protein thereof into the gesicle system, as described in Campbell, et al., Molecular Therapy, 27 (2019): 151-163, which is incorporated herein by reference in its entirety.

Engineered Virus-Like Particle (eVLP)

A further aspect of the disclosure is directed to modular and specific expansion of tropism of an engineered virus-like particle (eVLP). In some embodiments, the eVLP is as described in Banskota et al. (′el/185 (2): 250-265 (2022). In some embodiments, the glycoprotein of the eVLP is replaced with a fusogen (e.g., an engineered VSV-G with decreased or substantially eliminated infectivity) and a targeting moiety (e.g., an antibody or antigen-binding molecule) that is separate and different from the fusogen, as described herein, resulting in expansion of the tropism of the eVLP system. In some embodiments, the eVLP system with expanded tropism is used to package and deliver a CRISPR-Cas protein or a fusion protein thereof (e.g., a base editor or prime editor). In some embodiments, the eVLP system with expanded tropism is used to package and deliver a ribonucleoprotein comprising a CRISPR-Cas protein or a fusion protein thereof (e.g., a base editor or prime editor), and a gRNA of the CRISPR-Cas protein. In some embodiments, the CRISPR-Cas protein or a fusion protein thereof is fused to a gag protein (e.g., ML Vgag) via a cleavable linker, wherein cleavage of the linker in the target cell exposes a nuclear localization signal (NLS) positioned between the linker and the CRISPR-Cas protein or fusion protein. In some embodiments, the fusion protein comprises (e.g., from 5′ to 3′) a gag protein (e.g., ML Vgag), one or more nuclear export signals (NESs), a cleavable linker, one or more nuclear localization signals (NLSs), and a CRISPR-Cas protein or a fusion protein thereof (e.g., a base editor or prime editor). In some embodiments, the CRISPR-Cas protein or a fusion protein thereof is fused to a first dimerizable domain capable of dimerization or heterodimerization with a second dimerizable domain fused to a membrane protein, wherein presence of a ligand facilitates said dimerization and enriches the CRISPR-Cas protein or a fusion protein thereof into the eVLP system.

### Methods for Delivery

Another aspect of the disclosure is directed to a method for targeted delivery of a cargo comprising administering a targeted delivery vehicle to a subject in need of the cargo, wherein the targeted delivery vehicle comprises: a lipid bilayer membrane, wherein the lipid bilayer membrane forms a vesicle; a fusogen embedded in the lipid bilayer membrane; a targeting moiety embedded in the lipid bilayer membrane, wherein the targeting moiety is separate and different from the fusogen; and a cargo within the vesicle.

In some embodiments, the fusogen is an envelope protein from a virus. In some embodiments, the envelope protein is modified to not have a targeting function.

In some embodiments, the virus is selected from the group consisting of genera Arenaviridae, Filoviridae, Orthomyxoviridae, Rhabdoviridae, Togaviridae, Matonaviridae, Hantaviridae, Bunyaviridae, Retroviridae, Coronaviridae, Bornaviridae and Orthomyxoviridae. In some embodiments, the virus is selected from the group consisting of Pichinde virus, Ebola virus, Dhori virus, Duvenhage lyssavirus, European bat 1 lyssavirus, Isfahan virus, Mokola virus, Rabies virus, Chikungunya virus, Eastern equine encephalitis virus, O'nyong'nyong virus, Rubella virus, Hantaan orthohantavirus, Dugbe virus, La Crosse virus, Influenza A virus, Quaranfil virus, Lassa mammarenavirus, Lymphocytic Choriomeningitis virus, Mammalian Bornavirus 1, Marburg virus, Feline immunodeficiency virus, Rabies virus, Arizona vesiculovirus, Eastern equine encephalitis virus, Semliki Forest virus, Hantaan orthohantavirus, Indiana vesiculovirus, Severe acute respiratory syndrome coronavirus, Severe acute respiratory syndrome coronavirus 2, Influenza A virus, Baboon endogenous virus, Vesicular Stomatitis Virus and Sindbis virus.

In some embodiments, the fusogen is a pH-dependent fusogen. In some embodiments, the pH-dependent fusogen is selected from the group consisting of Sindbis Virus E2 protein, Vesicular Stomatitis Virus G protein, Cocal Virus G protein, and Chikungunya Virus E2 protein.

In some embodiments, the fusogen is a Vesicular Stomatitis Virus G (VSV-G) protein. In some embodiments, the VSV-G protein comprises a sequence that is at least 80%, 85%, 90%, 95%, 99% or more identical to SEQ ID NO: 2, or the VSV-G protein is encoded by a nucleic acid sequence that is at least 80%, 85%, 90%, 95%, 99% or more identical to SEQ ID NO: 1. In some embodiments, the VSV-G protein comprises at least one nonconservative point mutation at a position selected from H8, K47, Y209, and R354. In some embodiments, the VSV-G protein comprises at least one mutation selected from H8A, K47Q, Y209A, and R354Q.

In some embodiments, the fusogen is the Cocal Virus G protein. In some embodiments, the Cocal Virus G protein comprises a sequence that is at least 80%, 85%, 90%, 95%, 99% or more identical to SEQ ID NO: 4, or the Cocal Virus G protein is encoded by a nucleic acid sequence that is at least 80%, 85%, 90%, 95%, 99% or more identical to SEQ ID NO: 3. In some embodiments, the Cocal Virus G protein comprises at least one nonconservative point mutation at a position selected from the group consisting of Q25, K64, Y226, and R371 of SEQ ID NO: 4. In some embodiments, the Cocal Virus G protein comprises at least one mutation selected from Q25A, K64Q, Y226A, and R371Q.

In some embodiments, the fusogen is a Chikungunya Virus E2 protein. In some embodiments, the Chikungunya Virus E2 protein comprises a sequence that is at least 80%, 85%, 90%, 95%, 99% or more identical to SEQ ID NO: 8. In some embodiments, the Chikungunya Virus E2 protein comprises at least one nonconservative point mutation at a position selected from the group consisting of W64, D71, T116, 1121, 1190, Y199, and I217 (according to the amino acid positions shown in SEQ ID NO: 8). In some embodiments, the fusogen is the Chikungunya Virus E2, wherein the Chikungunya Virus E2 protein comprises at least one mutation selected from the group consisting of D71A, 1121A, 1190A, Y199A, and I217A.

In some embodiments, the fusogen comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

In some embodiments, the targeting moiety comprises a binding domain specific for a target cell of interest. In some embodiments, the binding domain comprises a receptor, an antibody, or an antigen-binding fragment. In some embodiments, the antibody fragment is selected from the group consisting of a Fab, a Fab′, a F(ab′)₂, an Fd, an Fv, a domain antibody, a complementarity determining region (CDR), a single chain variable fragment antibody (scFv), a maxibody, a minibody, an intrabody, a diabody, a triabody, a tetrabody, a variable domain of new antigen receptor (v-NAR) and a bispecific scFv (bis-scFv).

In some embodiments, the targeting moiety comprises a tag, and wherein the binding domain is attached to the targeting domain through the tag. In some embodiments, the tag is selected from the group consisting of a SNAP tag, a biotin tag, an Isopeptag, a Spy Tag, a SpyCatcher tag, a SnoopTag, a SnoopTagJr, a SnoopCatcher tag, a DogTag, a DogCatcher tag, a Gluthatione-S-transferase tag, a CLIP tag, a Protein A tag, a Protein G tag, a Protein AG tag, a GFP tag, an HA tag, a FLAG tag and a HiBiT-tag.

In some embodiments, the targeting moiety comprises a transmembrane domain selected from the group consisting of a Vesicular Stomatitis Virus G C terminal domain (VSVG-CTD), a transmembrane domain of Beta-2 microglobulin (B2M), a transmembrane domain of Human Leukocyte Antigen-A (HLA-A), and a transmembrane domain of platelet derived growth factor receptor beta (PDGFRB-TM).

In some embodiments, the target cell of interest is a mammalian cell. In some embodiments, the target cell of interest is a cancer cell. In some embodiments, the targeted delivery vehicle delivers the cargo to a B cell, a CD4+ T cell, a CD8+ T cell, a lung cell, a colorectal cell, a hematopoietic stem cell, a muscle cell, a cardiac cell, a hepatocyte, a monocyte, a macrophage or a neuronal cell.

In some embodiments, the cargo comprises a deoxyribonucleic acid (DNA), a ribonucleic acid (RNA), a protein, a ribonucleoprotein (RNP) or a combination thereof. In some embodiments, the cargo comprises an expression vector, a gene-editing tool, or a gene-silencing tool. In some embodiments, the cargo comprises Cre mRNA or Cas9-RNP.

In some embodiments, the targeted delivery vehicle is a pseudotyped lentiviral vector, a selective endogenous encapsidation for cellular delivery system (SEND), a nanoblade, an engineered virus-like particle (eVLP), or a gesicle.

In some embodiments, the targeted delivery vehicle is administered locally or systemically.

### Expanded Example Cargo Molecules

The delivery vesicles described herein may be used and further comprise a number of different cargo molecules for delivery. Representative cargo molecules may include, but are not limited to, nucleic acids, polynucleotides, proteins, polypeptides, polynucleotide/polypeptide complexes, small molecules, sugars, or a combination thereof. Cargos that can be delivered in accordance with the systems and methods described herein include, but are not necessarily limited to, biologically active agents, including, but not limited to, therapeutic agents, imaging agents, and monitoring agents. A cargo may be an exogenous material or an endogenous material.

### Polynucleotides

In some embodiments, the cargo is a cargo polynucleotide. As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA, including but not limited to, tRNA (transfer RNA), snRNA (small nuclear RNA), IRNA (ribosomal RNA), anti-sense RNA, RNAi (RNA interference construct), siRNA (short interfering RNA), microRNA (miRNA), or ribozymes, aptamers, guide RNA (gRNA), or coding mRNA (messenger RNA).

In some embodiments, the cargo polynucleotide is DNA. In some embodiments, the cargo polynucleotide is RNA. In some embodiments, the cargo polynucleotide is a polynucleotide (a DNA or an RNA) that encodes an RNA and/or a polypeptide. As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Interference RNAs

In certain example embodiments, the one or more polynucleotides may encode one or more interference RNAs. Interference RNAs are RNA molecules capable of suppressing gene expressions. Example types of interference RNAs include small interfering RNA (siRNA), microRNA (miRNA), and short hairpin RNA (shRNA).

In certain example embodiments, the interference RNA may be a small interfering RNA (siRNA). siRNA molecules are capable of inhibiting target gene expression by interfering RNA. siRNAs may be chemically synthesized, or may be obtained by in vitro transcription, or may be synthesized in vivo in target cell. siRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode small hairpin RNAs (shRNAs). In shRNAs the antiparallel strands that form siRNA are connected by a loop or hairpin region.

The interference RNA (e.g., siRNA) may suppress expression of genes to promote long-term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway. TGFβ pathway genes may comprise one or more of ACVR1, ACVRIC, ACVR2A, ACVR2B, ACVRL1, AMH, AMHR2, BMP2, BMP4, BMP5, BMP6, BMP7, BMP8A, BMP8B, BMPRIA, BMPRIB, BMPR2, CDKN2B, CHRD, COMP, CREBBP, CUL1, DCN, E2F4, E2F5, EP300, FST, GDF5, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2R1B, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

In some embodiments, the cargo polynucleotide is an RNAi molecule, antisense molecule, and/or a gene silencing oligonucleotide or a polynucleotide that encodes an RNAi molecule, antisense molecule, and/or gene silencing oligonucleotide.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, and short-hairpin RNA (shRNA). Commercially available programs and tools are available to design the nucleotide sequence of gene silencing oligonucleotides for a desired gene, based on the gene sequence and other information available to one of ordinary skill in the art.

### Therapeutic Polynucleotides

In some embodiments, the cargo molecule is a therapeutic polynucleotide. Therapeutic polynucleotides are those that provide a therapeutic effect when delivered to a recipient cell. The polynucleotide can be a toxic polynucleotide (a polynucleotide that when transcribed or translated results in the death of the cell) or polynucleotide that encodes a lytic peptide or protein. In embodiments, delivery vesicles having a toxic polynucleotide as a cargo molecule can act as an antimicrobial or antibiotic. This is discussed in greater detail elsewhere herein. In some embodiments, the cargo molecule can be exogenous to the producer cell and/or a first cell. In some embodiments, the cargo molecule can be endogenous to the producer cell and/or a first cell. In some embodiments, the cargo molecule can be exogenous to the recipient cell and/or a second cell. In some embodiments, the cargo molecule can be endogenous to the recipient cell and/or second cell.

As described herein the cargo polynucleotide can be any polynucleotide endogenous or exogenous to the eukaryotic cell. For example, the cargo polynucleotide can be a polynucleotide residing in the nucleus of the eukaryotic cell. The cargo polynucleotide can be a sequence coding a gene product (e.g., a protein) or a non-coding sequence (e.g., a regulatory polynucleotide).

In some embodiments, the cargo polynucleotide is a DNA or RNA (e.g., a mRNA) vaccine.

### Aptamers

In certain example embodiments, the polynucleotide may be an aptamer. In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′—NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present invention may utilize any aptamer containing any modification as described herein.

In certain other example embodiments, the polynucleotide may be a ribozyme or other enzymatically active polynucleotide.

### Biologically Active Agents

In some embodiments, the cargo is a biologically active agent. Biologically active agents include any molecule that induces, directly or indirectly, an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic acid cargo. Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein and the like. Reporter probes include photo-emitting compounds, such as phosphors, radioactive moieties and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors and regulator proteins used as recognition elements, which can be used either in whole cells or isolated and used independently (D'Souza, 2001, Biosensors and Bioelectronics 16:337-353).

One or two or more different cargoes may be delivered by the delivery particles described herein.

In some embodiments, the cargo may be linked to one or more envelope proteins by a linker, as described elsewhere herein. A suitable linker may include, but is not necessarily limited to, a glycine-serine linker. In some embodiments, the glycine-serine linker is (GGS)3 (SEQ ID NO: 48).

In some embodiments, the cargo comprises a ribonucleoprotein. In specific embodiments, the cargo comprises a genetic modulating agent.

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar terms. As used herein, “modulating” or “to modulate” generally means either reducing or inhibiting the activity of a target or antigen, or alternatively increasing the activity of the target or antigen, as measured using a suitable in vitro, cellular or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

### Gene Modifying Systems

In some embodiments, the cargo is a polynucleotide modifying system or component(s) thereof. In some embodiments the polynucleotide modifying system is a gene modifying system. In some embodiments, the gene modifying system is or is composed of a gene modulating agent. In some embodiments, the genetic modulating agent may comprise one or more components of a polynucleotide modification system (e.g., a gene editing system) and/or polynucleotides encoding thereof.

In some embodiments, the gene editing system may be an RNA-guided system or other programmable nuclease system. In some embodiments, the gene editing system is an IscB system. In some embodiments, the gene editing system may be a CRISPR-Cas system.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

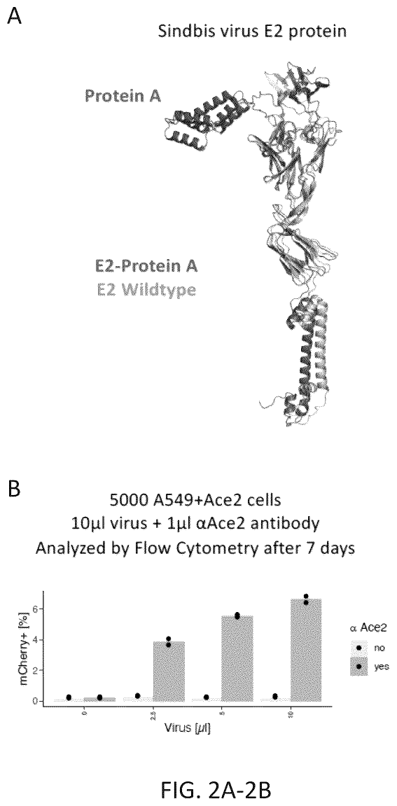

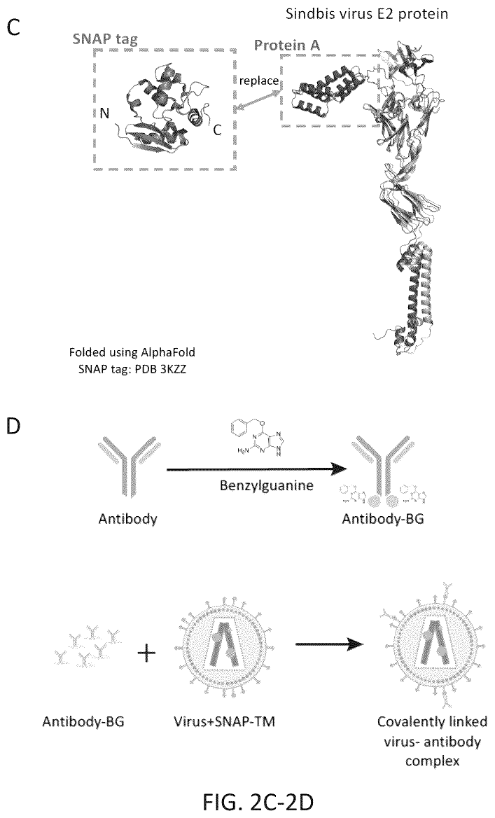

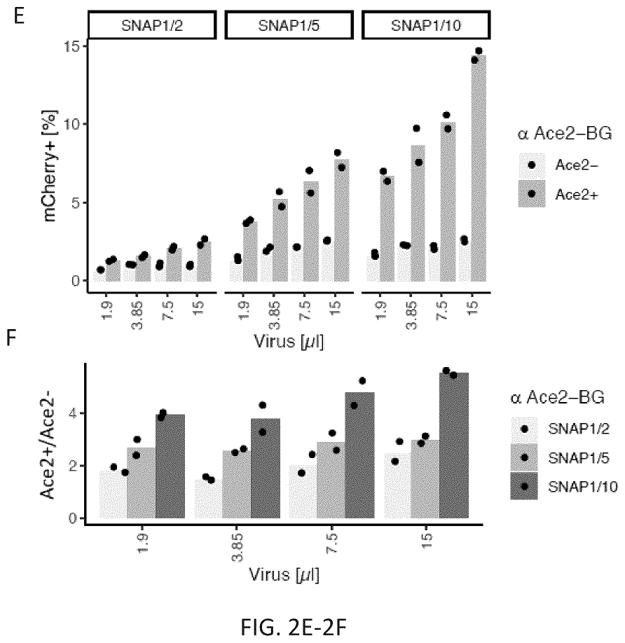

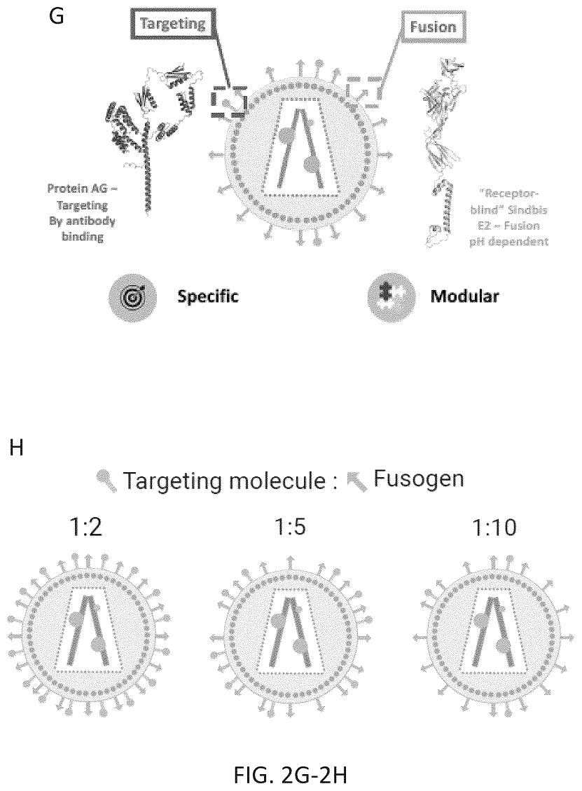

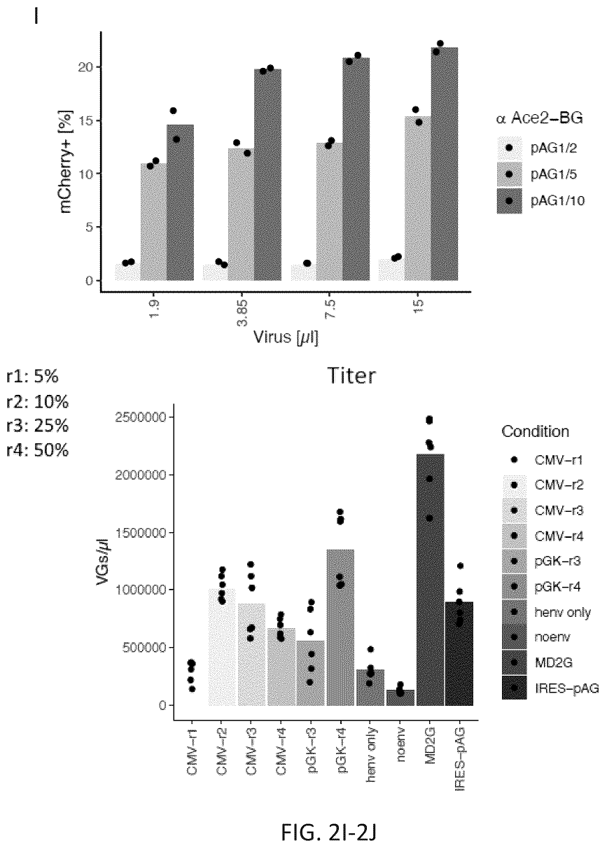

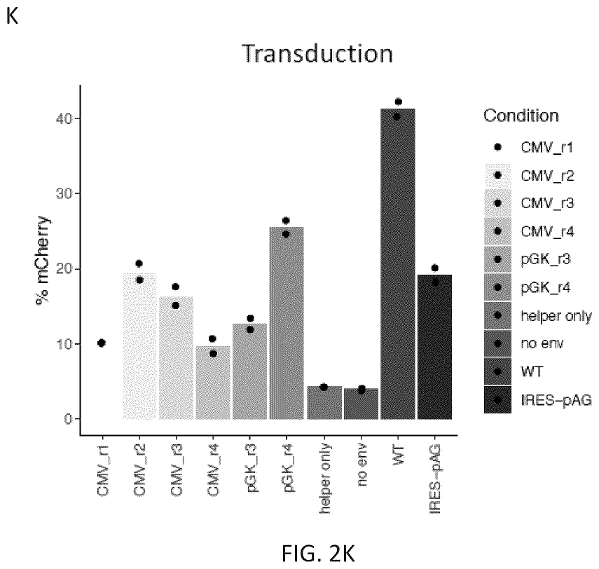

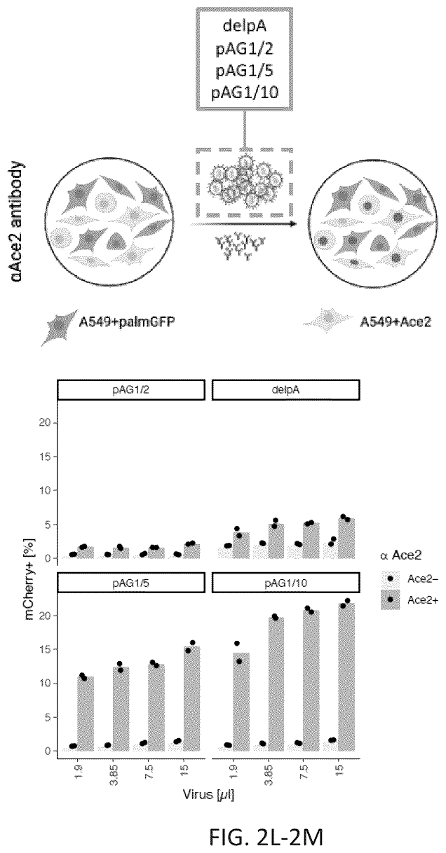

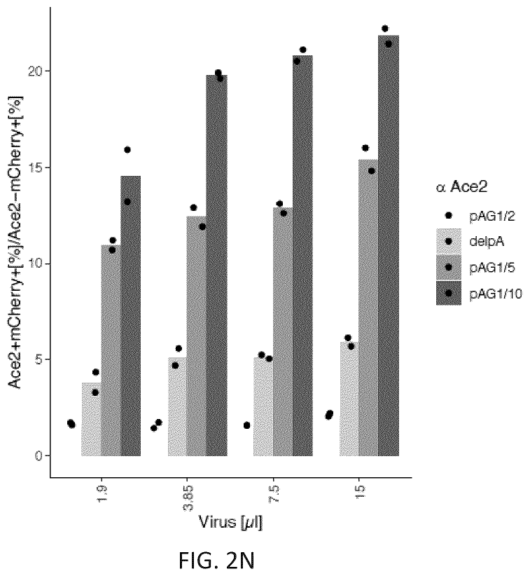

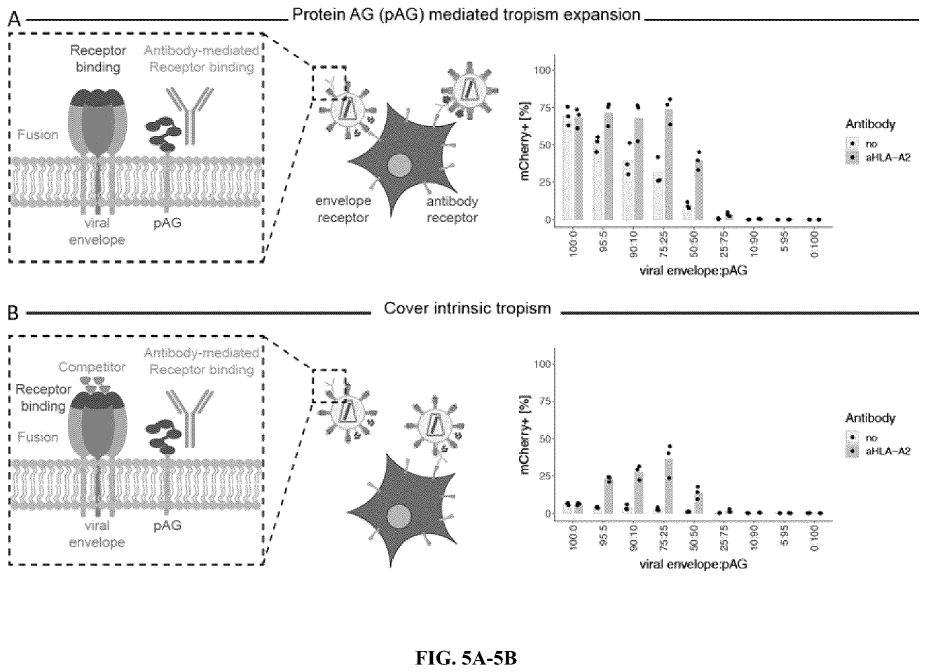

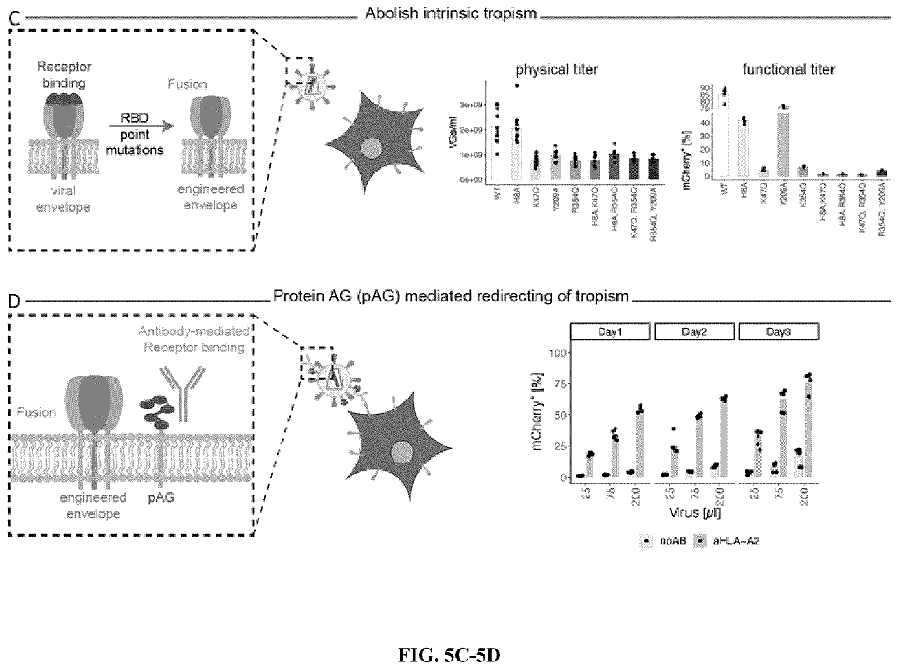

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas 10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374:20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36 (4) 702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some embodiments, the target sequence may be a sequence within an RNA molecule selected from the group consisting of mRNA, pre-mRNA, and rRNA. In some embodiments, the target sequence may be a sequence within an RNA molecule selected from the group consisting of ncRNA, and lncRNA. In some embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nucleotides (nt). In certain embodiments, the spacer length of the guide RNA is at least 15 nt. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333], which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16 (4): 504-517. Table 3 (from Gleditzsch et al. 2019) below shows several Cas polypeptides and the PAM sequence they recognize.

TABLE 3
Example PAM Sequences

| | |
|---|---|
| Cas Protein | PAM Sequence |
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a specific embodiment, the CRISPR effector protein may recognize a 3′ PAM. In certain embodiments, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3): 733-740; Atschul et al. 1990. J. Mol. Biol. 215:403-410; Biswass et al. 2013 RNA Biol. 10:817-827; and Grissa et al. 2007. Nucleic Acid Res. 35: W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16 (4): 504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16 (4): 504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 13) or PKKKRKVEAS (SEQ ID NO: 14); the NLS from nucleoplasmin (e.g., the nucleoplasmin bipartite NLS with the sequence KRPAATKKAGQAKKKK (SEQ ID NO: 15)); the c-myc NLS having the amino acid sequence PAAKRVKLD (SEQ ID NO: 16) or RQRRNELKRSP (SEQ ID NO: 17); the hRNPA1 M9 NLS having the sequence NQSSNFGPMKGGNFGGRSSGPYGGGGQYFAKPRNQGGY (SEQ ID NO: 18); the sequence RMRIZFKNKGKDTAELRRRRVEVSVELRKAKKDEQILKRRNV (SEQ ID NO: 19) of the IBB domain from importin-alpha; the sequences VSRKRPRP (SEQ ID NO: 20) and PPKKARED (SEQ ID NO: 21) of the myoma T protein; the sequence PQPKKKPL (SEQ ID NO: 22) of human p53; the sequence SALIKKKKKMAP (SEQ ID NO: 23) of mouse c-abl IV; the sequences DRLRR (SEQ ID NO: 24) and PKQKKRK (SEQ ID NO: 25) of the influenza virus NS1; the sequence RKLKKKIKKL (SEQ ID NO: 26) of the Hepatitis virus delta antigen; the sequence REKKKFLKRR (SEQ ID NO: 27) of the mouse Mx1 protein; the sequence KRKGDEVDGVDEVAKKKSKK (SEQ ID NO: 28) of the human poly(ADP-ribose) polymerase; and the sequence RKCLQAGMNLEARKTKK (SEQ ID NO: 29) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In some embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

It will be appreciated that NLS and NES described herein with respect to Cas proteins can be used with other cargos, in particularly, gene modifying agents herein, and other proteins that can benefit from translocation in or out of a nuclease of a cell, such as a target cell.

### Donor Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 1 1, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154 (6): 1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33 (2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19 (12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19 (12): 770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358:1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576:149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576:149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

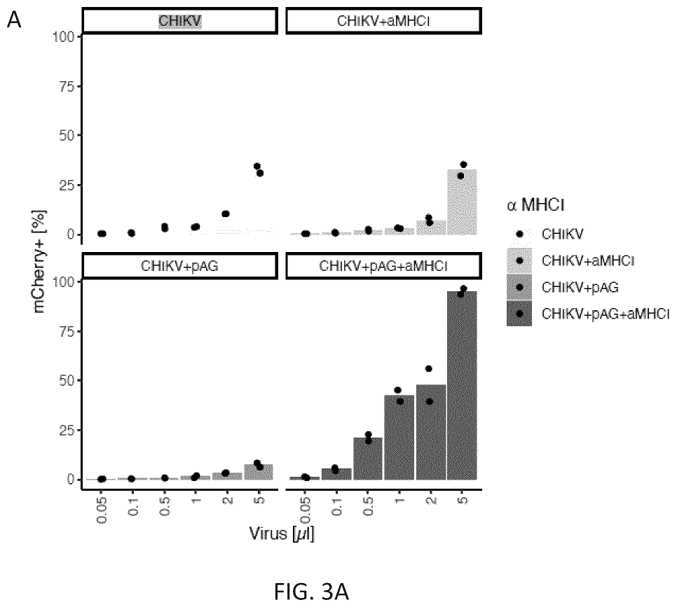

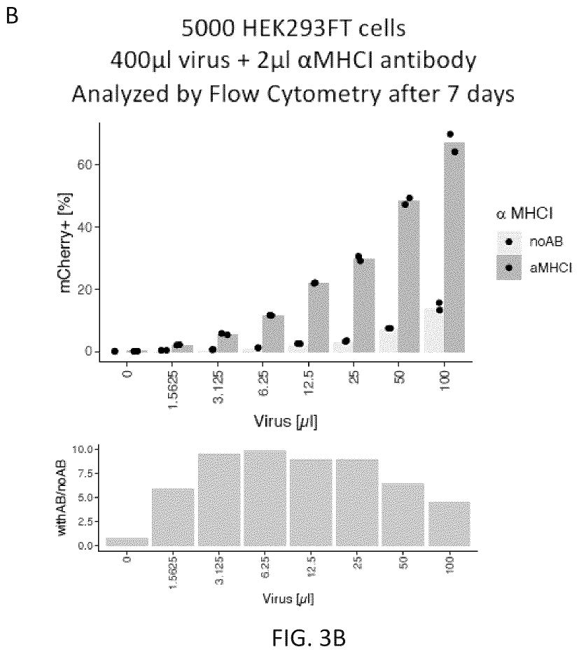

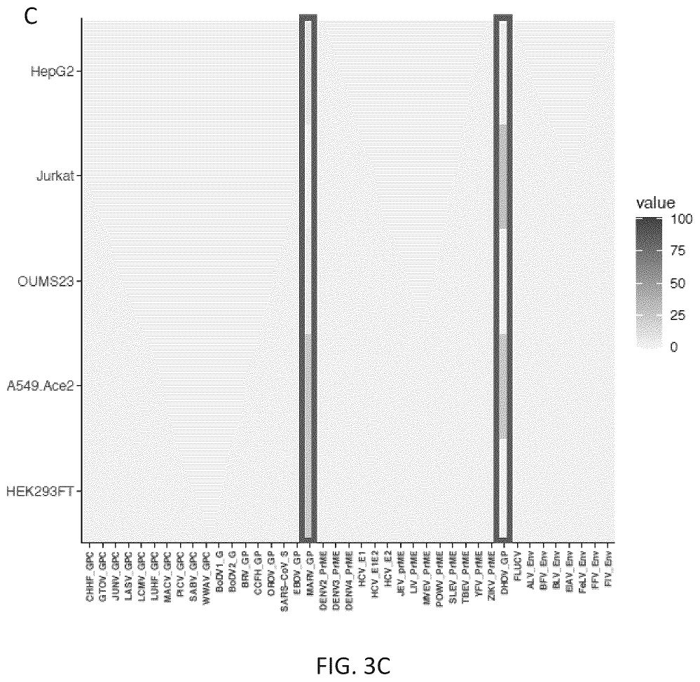

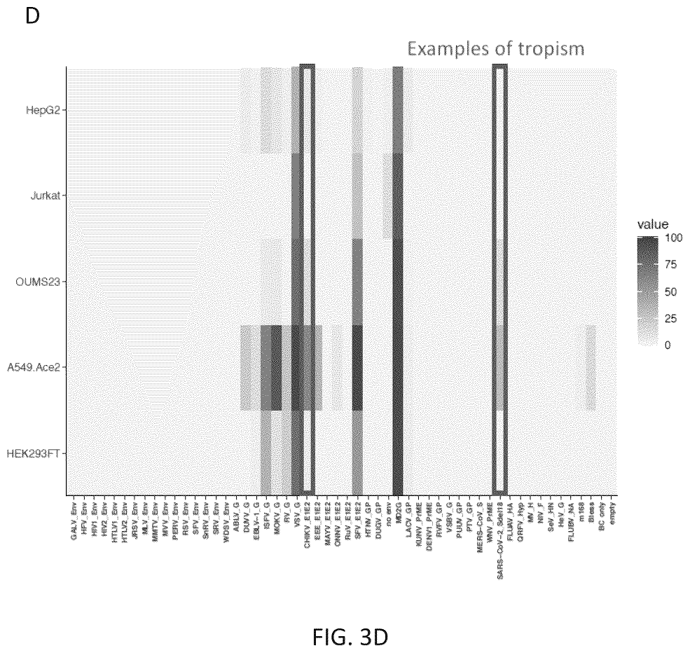

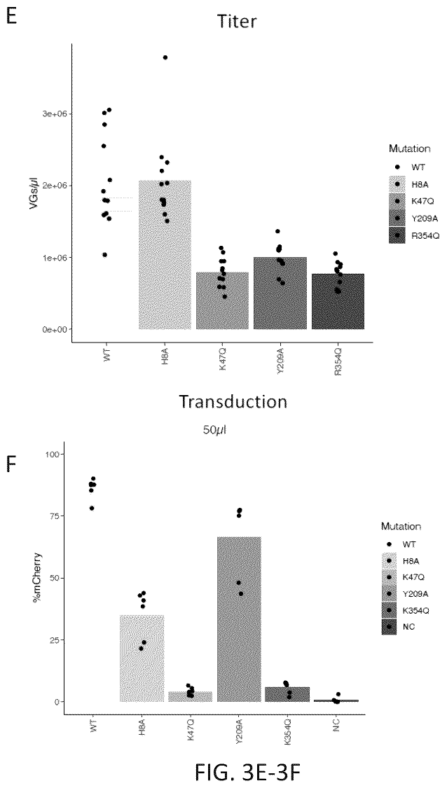

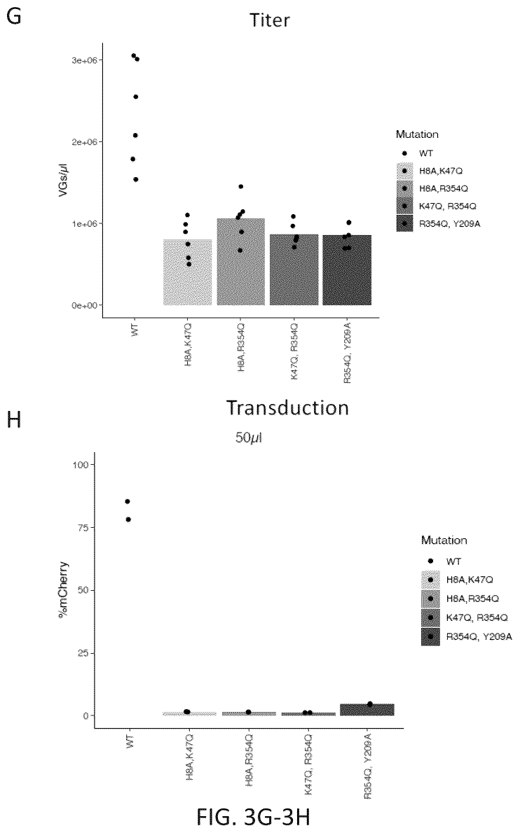

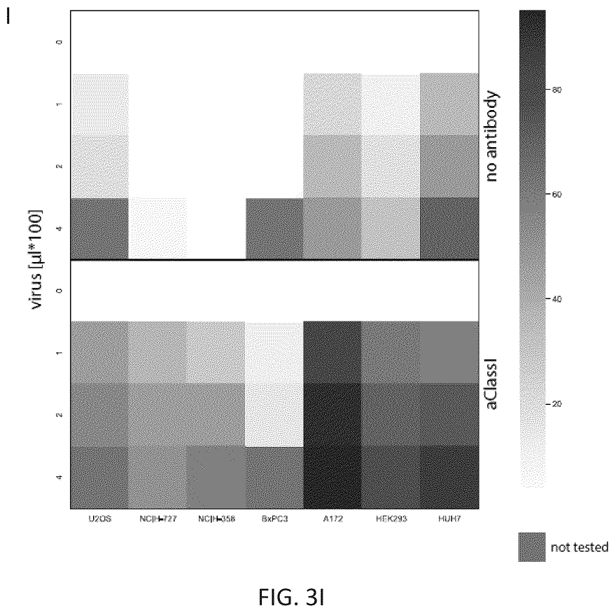

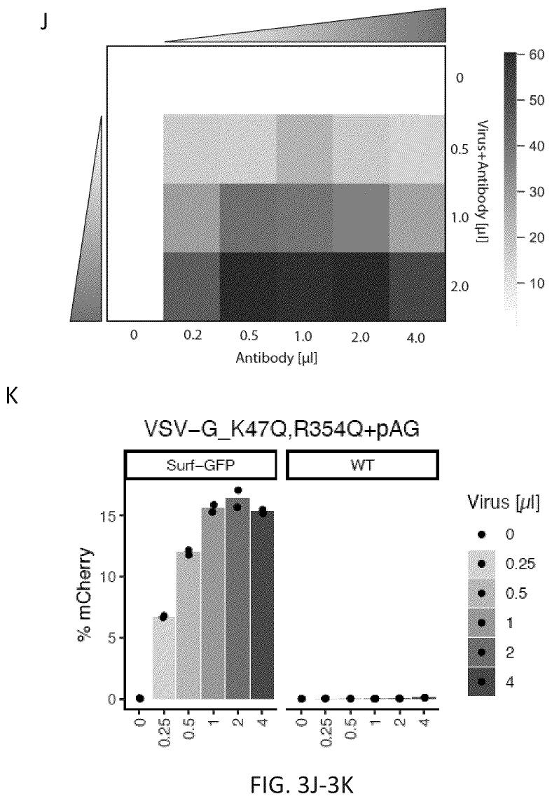

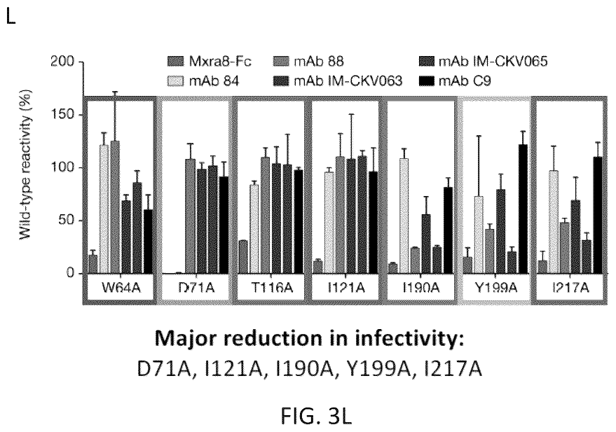

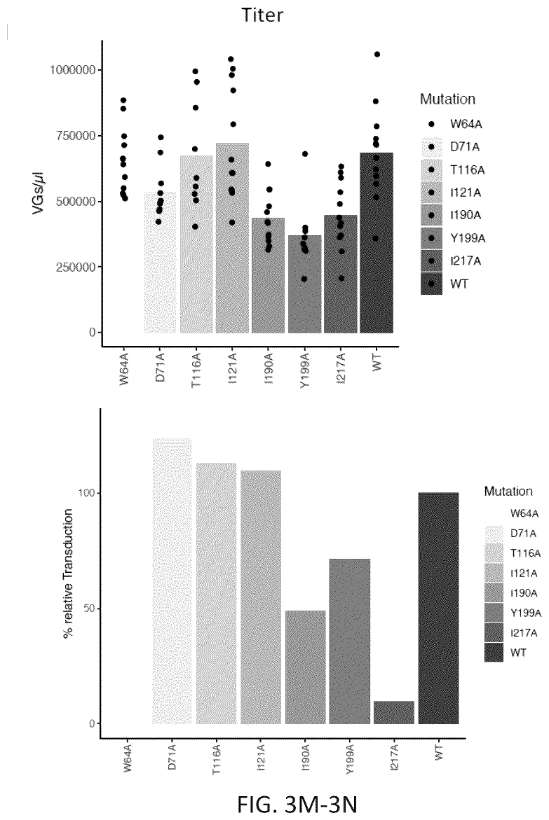

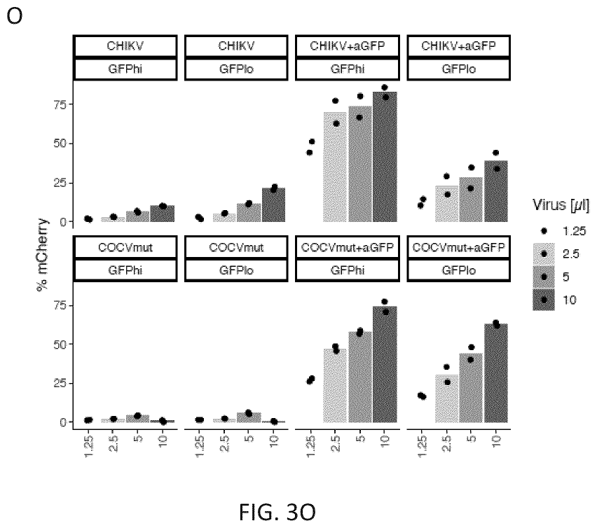

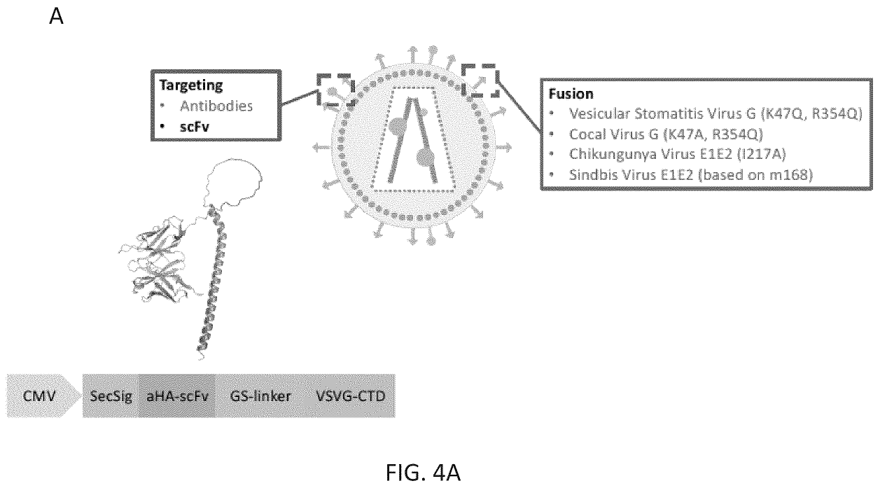

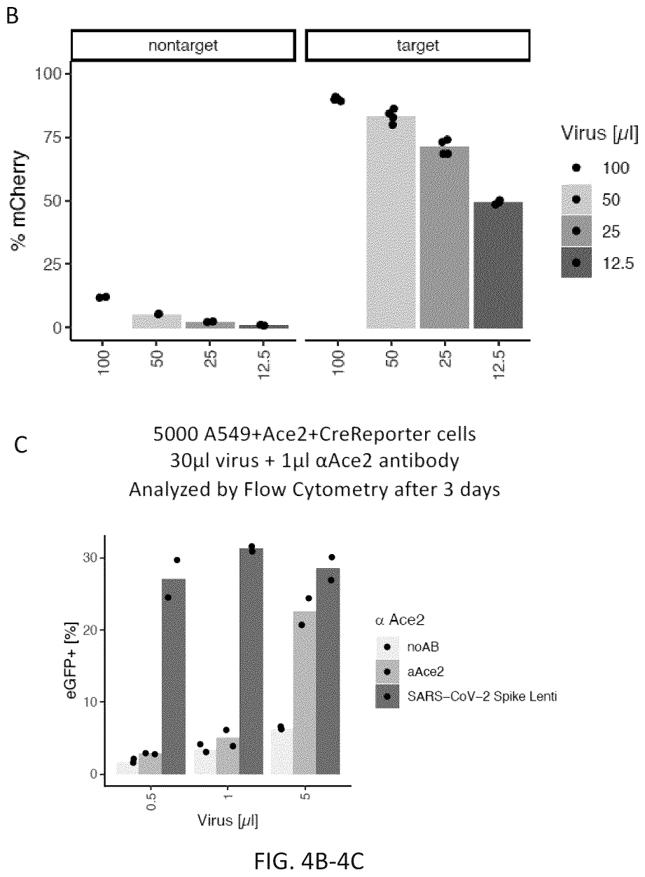

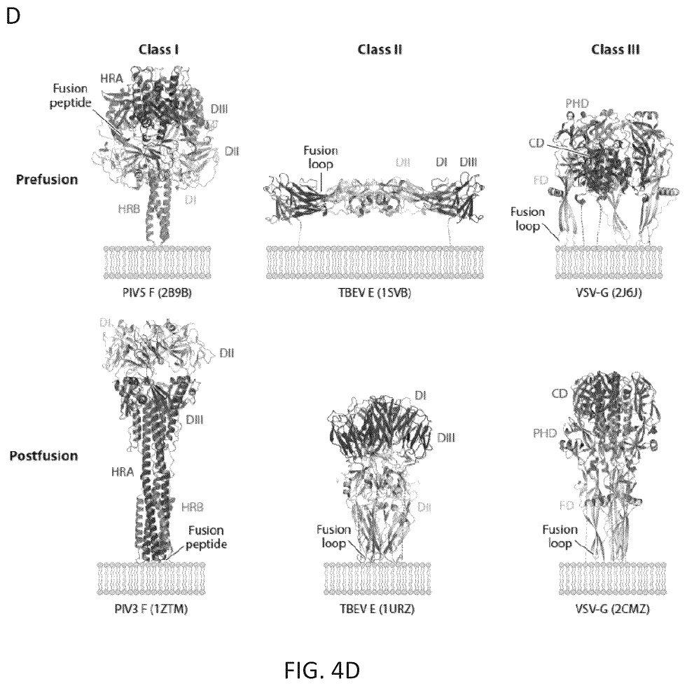

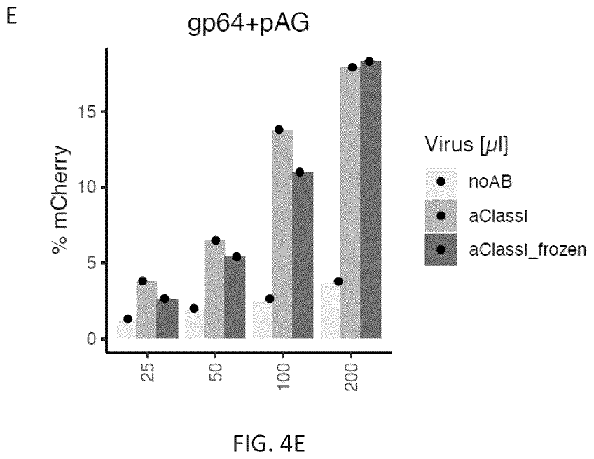

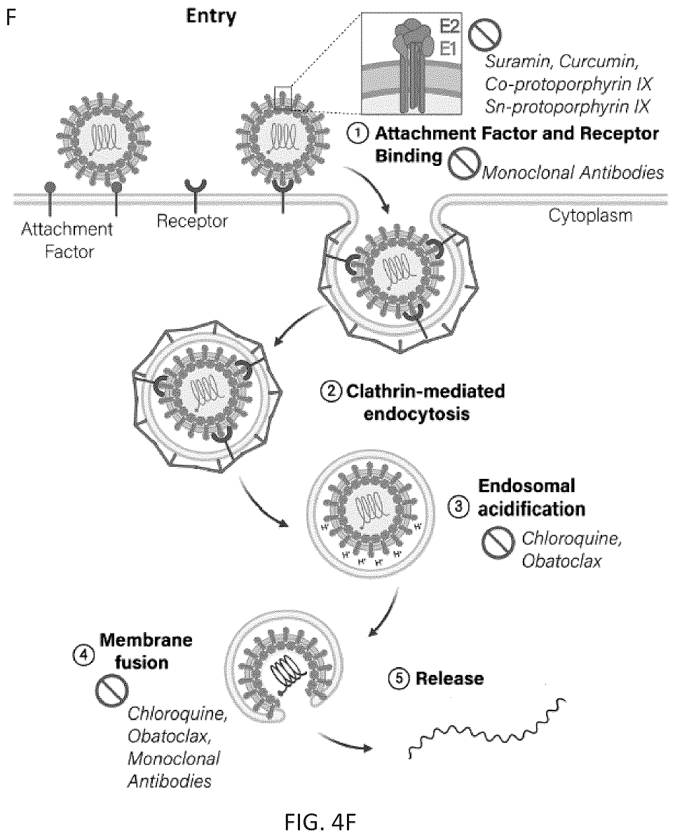

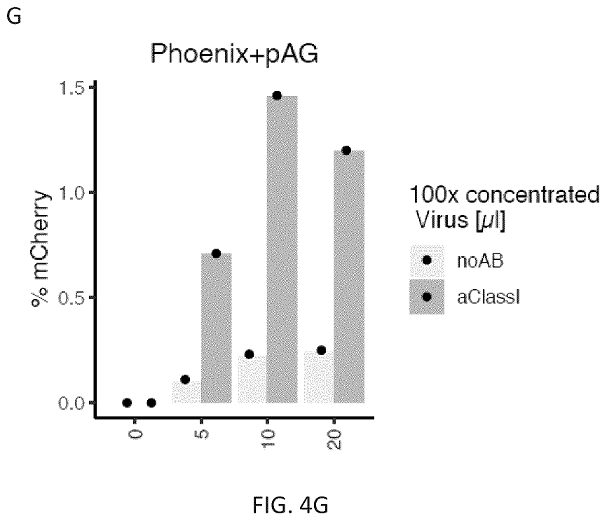

In some embodiments, the prime editing system can be a PE1 system or variant thereof, a PE2 system or variant thereof, or a PE3 (e.g., PE3, PE3b) system. See e.g., Anzalone et al. 2019. Nature. 576:149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi: 10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### IscBs

In some embodiments, the nucleic acid-guided nucleases herein may be IscB proteins. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated.

In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198 (5): 797-807. doi: 10.1128/JB.00783-15, which is incorporated by reference herein in its entirety.

In some embodiments, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises an N-terminal X domain, a RuvC domain (e.g., including a RuvC-I, RuvC-II, and RuvC-III subdomains), a Bridge Helix domain, an HNH domain, and a C-terminal Y domain.

In some embodiments, the nucleic acid-guided nucleases may have a small size. For example, the nucleic acid-guided nucleases may be no more than 50, no more than 100, no more than 150, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 amino acids in length.

In some examples, the IscB protein shares at least 80%, at least 85%, at least 90%, at least 95%, at least 99%, or 100% sequence identity with a IscB protein selected from Table 4.

TABLE 3

| No. | Proteins | Sequences |
|---|---|---|
| 1 | IscB(−HNH) | MSTDATLIRTTPSHAEADATDTLVATPLMPPRRVISPWPGPGE |
| | EFH81386 | GQSLMRIPVVDIRGMALMPCTPAKARHLLKSGNARPKRNKL |
| | | GLFYVQLSYEQEPDNQSLVAGVDPGSKFEGLSVVGTKDTVL |
| | | NLMVEAPDHVKGAVQTRRTMRRARRQRKWRRPKRFHNRLN |
| | | RMQRIPPSTRSRWEAKARIVAHLRTILPFTDVVVEDVQAVTR |
| | | KGKGGTWNGSFSPVQVGKEHLYRLLRAMGLTLHLREGWQT |
| | | KELREQHGLKKTKSKSKQSFESHAVDSWVLAASISGAEHPTC |
| | | TRLWYMVPAILHRRQLHRLQASKGGVRKPYGGTRSLGVKRG |
| | | TLVEHKKYGRCTVGGVDRKRNTISLHEYRTNTRLTQAAKVE |
| | | TCRVLTWLSWRSWLLRGKRTSSKGKGSHSS (SEQ ID NO: 30) |
| 2 | IscB(+HNH) | MQPAKQQNWVFQINGDKQPLDMINPGRCRELQNRGKLASFR |
| | TAE54104.1 | RFPYVVIQQQTIENPQTKEYILKIDPGSQWTGFAIQCGNDILFR |
| | | AELNHRGEAIKFDLVKRAWFRRGRRSRNLRYRKKRLNRAKP |
| | | EGWLAPSIRHRVLTVETWIKRFMRYCPIAWIEIEQVRFDTQKL |
| | | ANPEIDGVEYQQGELQGYEVREYLLQKWGRKCAYCGTENVP |
| | | LEVEHIQSKSKGGSSRIGNLTLACHVCNVKKGNLDVRDFLAK |
| | | SPDILNQVLENSTKPLKDAAAVNSTRYAIVKMAKSICENVKC |
| | | SSGARTKMNRVRQGLEKTHSLDAACVGESGASIRVLTDRPLL |
| | | ITCKGHGSRQSIRVNASGFPAVKNAKTVFTHIAAGDVVRFTIG |
| | | KDRKKAQAGTYTARVKTPTPKGFEVLIDGAR |
| | | ISLSTMSNVVFVHRSDGYGYEL (SEQ ID NO: 31) |
| 3 | IscB(+HNH) | MAVFVIDKHKRPLMPCSEKRARLLLERGRAVVHRQVPFV |
| | WP_038093640.1 | IRLKDRTVQHSAVQPLRVALDPGSRATGMALVREKNTVD |
| | | TGTGEVYRERIALNLFELVHRGHRIREQLDQRRNFRRRRR |
| | | GANLRYRAPRFDNRRRPPGWLAPSLQHRVDTTMAWVRR |
| | | LCRWAPASAIGIETVRFDTQRLQNPEISGVEYQQGALAGC |
| | | EVREYLLEKWGRKCAYCGAENVPLEIEHIVPKSRGGSDRV |
| | | SNLALACRACNQAKGNRDVRAFLADQPERLARILAQAKA |
| | | PLKDAAAVNATRWALYRALVDTGLPVEAGTGGRTKWNR |
| | | TRLGLPKTHALDALCVGQVDQVRHWRVPVLGIRCAGRGS |
| | | YRRTRLTRHGFPRGYLTRNKSAFGFQTGDLIRAVVTKGK |
| | | KAGTYLGRIAIRASGSFNIQTPMGVVQGIHHRFCTLLQRA |
| | | DGYGYFVQPKPTEAALSSPRLKAGVSSAGN (SEQ ID NO: |
| | | 32) |
| 4 | IscB(+HNH) | MTTNVVFVIDTNQKPLQPCSAAVARKLLLRGKAAMFRRY |
| | WP_052490348.1 | PAVIILKKEVDSVGKPKIELRIDPGSKYTGFALVDSKDNAD |
| | | FIIWGTELEHRGAAICKELTKRSAIRRSRRNRKTRYRKKRF |
| | | ERRKPEGWLAPSLQHRVDTTLTWVKRICKFVPIMSISVEQ |
| | | VKFDLQKLENSDIQGIEYQQGTLAGYTLREALLEHWGRK |
| | | CAYCDVENVFLEIEHIYPKSKGGSDKFSNLTLACHKCNIN |
| | | KGNKSIDEFLLSDHKRLEQIKLHQKKTLKDAAAVNATRK |
| | | KLVTTLQEKTFLNVLVSDGASTKMTRLSSSLAKRHWIDA |
| | | GCVNTTLIVILKTLQPLQVKCNGHGNKQFVTMDAYGFPR |
| | | KSYEPKKVRKDWKAGDIIRVTKKDGTMLMGRVKKAAKK |
| | | LVYIPFGGKEASFSSENAKAIHRSDGYRYSFAAIDSELLQK |
| | | MAT (SEQ ID NO: 33) |
| 5 | IscB(+HNH) | MPNKYAFVLDSKGKLLDPTKSKKAWYLIRKGKASLVEEY |
| | WP_015325818.1 | PLIIKLKREVPKDQVNSDKLILGIDDGTKKVGFALVQKCQ |
| | | TKNKVLFKAVMEQRQDVSKKMEERRGYRRYRRSHKRYR |
| | | PARFDNRSSSKRKGRIPPSILQKKQAILRVVNKLKKYIRID |
| | | KIVLEDVSIDIRKLTEGRELYNWEYQESNRLDENLRKATL |
| | | YRDDCTCQLCGTTETMLHAHHIMPRRDGGADSIYNLITLC |
| | | KACHKDKVDNNEYQYKDQFLAIIDSKELSDLKSASHVMQ |
| | | GKTWLRDKLSKIAQLEITSGGNTANKRIDYEIEKSHSNDAI |
| | | CTTGLLPVDNIDDIKEYYIKPLRKKSKAKIKELKCFRQRDL |
| | | VKYTKRNGETYTGYITSLRIKNNKYNSKVCNFSTLKGKIF |
| | | RGYGFRNLTLLNRPKGLMIV (SEQ ID NO: 34) |
| 6 | sp\|G3ECR1\| | MLFNKCIIISINLDFSNKEKCMTKPYSIGLDIGTNSVGWAVI |
| | CAS9_STRTR | TDNYKVPSKKMKVLGNTSKKYIKKNLLGVLLFDSGITAE |
| | | GRRLKRTARRRYTRRRNRILYLQEIFSTEMATLDDAFFQR |
| | | LDDSFLVPDDKRDSKYPIFGNLVEEKVYHDEFPTIYHLRK |
| | | YLADSTKKADLRLVYLALAHMIKYRGHFLIEGEFNSKNN |
| | | DIQKNFQDFLDTYNAIFESDLSLENSKQLEEIVKDKISKLE |
| | | KKDRILKLFPGEKNSGIFSEFLKLIVGNQADFRKCFNLDEK |
| | | ASLHFSKESYDEDLETLLGYIGDDYSDVFLKAKKLYDAIL |
| | | LSGFLTVTDNETEAPLSSAMIKRYNEHKEDLALLKEYIRNI |
| | | SLKTYNEVFKDDTKNGYAGYIDGKTNQEDFYVYLKNLLA |
| | | EFEGADYFLEKIDREDFLRKQRTFDNGSIPYQIHLQEMRAI |
| | | LDKQAKFYPFLAKNKERIEKILTFRIPYYVGPLARGNSDFA |
| | | WSIRKRNEKITPWNFEDVIDKESSAEAFINRMTSFDLYLPE |
| | | EKVLPKHSLLYETFNVYNELTKVRFIAESMRDYQFLDSKQ |
| | | KKDIVRLYFKDKRKVTDKDIIEYLHAIYGYDGIELKGIEKQ |
| | | FNSSLSTYHDLLNIINDKEFLDDSSNEAIIEEIIHTLTIFEDRE |
| | | MIKQRLSKFENIFDKSVLKKLSRRHYTGWGKLSAKLINGI |
| | | RDEKSGNTILDYLIDDGISNRNFMQLIHDDALSFKKKIQKA |
| | | QIIGDEDKGNIKEVVKSLPGSPAIKKGILQSIKIVDELVKVM |
| | | GGRKPESIVVEMARENQYTNQGKSNSQQRLKRLEKSLKE |
| | | LGSKILKENIPAKLSKIDNNALQNDRLYLYYLQNGKDMYT |
| | | GDDLDIDRLSNYDIDHIIPQAFLKDNSIDNKVLVSSASNRG |
| | | KSDDFPSLEVVKKRKTFWYQLLKSKLISQRKFDNLTKAER |
| | | GGLLPEDKAGFIQRQLVETRQITKHVARLLDEKFNNKKDE |
| | | NNRAVRTVKIITLKSTLVSQFRKDFELYKVREINDFHHAH |
| | | DAYLNAVIASALLKKYPKLEPEFVYGDYPKYNSFRERKSA |
| | | TEKVYFYSNIMNIFKKSISLADGRVIERPLIEVNEETGESV |
| | | WNKESDLATVRRVLSYPQVNVVKKVEEQNHGLDRGKPK |
| | | GLFNANLSSKPKPNSNENLVGAKEYLDPKKYGGYAGISNS |
| | | FAVLVKGTIEKGAKKKITNVLEFQGISILDRINYRKDKLNF |
| | | LLEKGYKDIELIIELPKYSLFELSDGSRRMLASILSTNNKRG |
| | | EIHKGNQIFLSQKFVKLLYHAKRISNTINENHRKYVENHK |
| | | KEFEELFYYILEFNENYVGAKKNGKLLNSAFQSWQNHSID |
| | | ELCSSFIGPTGSERKGLFELTSRGSAADFEFLGVKIPRYRDY |
| | | TPSSLLKDATLIHQSVTGLYETRIDLAKLGEG (SEQ ID NO: |
| | | 35) |
| 7 | sp\|J7RUA5\| | MKRNYILGLDIGITSVGYGIIDYETRDVIDAGVRLFKEANV |
| | CAS9_STAAU | ENNEGRRSKRGARRLKRRRRHRIQRVKKLLFDYNLLTDH |
| | | SELSGINPYEARVKGLSQKLSEEEFSAALLHLAKRRGVHN |
| | | VNEVEEDTGNELSTKEQISRNSKALEEKYVAELQLERLKK |
| | | DGEVRGSINRFKTSDYVKEAKQLLKVQKAYHQLDQSFID |
| | | TYIDLLETRRTYYEGPGEGSPFGWKDIKEWYEMLMGHCT |
| | | YFPEELRSVKYAYNADLYNALNDLNNLVITRDENEKLEY |
| | | YEKFQIIENVFKQKKKPTLKQIAKEILVNEEDIKGYRVTST |
| | | GKPEFTNLKVYHDIKDITARKEIIENAELLDQIAKILTIYQS |
| | | SEDIQEELTNLNSELTQEEIEQISNLKGYTGTHNLSLKAINL |
| | | ILDELWHTNDNQIAIFNRLKLVPKKVDLSQQKEIPTTLVDD |
| | | FILSPVVKRSFIQSIKVINAIIKKYGLPNDIIIELAREKNSKDA |
| | | QKMINEMQKRNRQTNERIEEIIRTTGKENAKYLIEKIKLHD |
| | | MQEGKCLYSLEAIPLEDLLNNPFNYEVDHIIPRSVSFDNSF |
| | | NNKVLVKQEENSKKGNRTPFQYLSSSDSKISYETFKKHIL |
| | | NLAKGKGRISKTKKEYLLEERDINRFSVQKDFINRNLVDT |
| | | RYATRGLMNLLRSYFRVNNLDVKVKSINGGFTSFLRRKW |
| | | KFKKERNKGYKHHAEDALIIANADFIFKEWKKLDKAKKV |
| | | MENQMFEEKQAESMPEIETEQEYKEIFITPHQIKHIKDFKD |
| | | YKYSHRVDKKPNRELINDTLYSTRKDDKGNTLIVNNLNG |
| | | LYDKDNDKLKKLINKSPEKLLMYHHDPQTYQKLKLIMEQ |
| | | YGDEKNPLYKYYEETGNYLTKYSKKDNGPVIKKIKYYGN |
| | | KLNAHLDITDDYPNSRNKVVKLSLKPYRFDVYLDNGVYK |
| | | FVTVKNLDVIKKENYYEVNSKCYEEAKKLKKISNQAEFIA |
| | | SFYNNDLIKINGELYRVIGVNNDLLNRIEVNMIDITYREYL |
| | | ENMNDKRPPRIIKTIASKTQSIKKYSTDILGNLYEVKSKKH |
| | | PQIIKKG (SEQ ID NO: 36) |
| 8 | Streptococcus_ | KYSIGLDIGTNSVGWAVITDEYKVPSKKFKVLGNTDRHSI |
| | pyogenes_ | KKNLIGALLFDSGETAEATRLKRTARRRYTRRKNRICYLQ |
| | SF370 | EIFSNEMAKVDDSFFHRLEESFLVEEDKKHERHPIFGNIVD |
| | | EVAYHEKYPTIYHLRKKLVDSTDKADLRLIYLALAHMIKF |
| | | RGHFLIEGDLNPDNSDVDKLFIQLVQTYNQLFEENPINASG |
| | | VDAKAILSARLSKSRRLENLIAQLPGEKKNGLFGNLIALSL |
| | | GLTPNFKSNFDLAEDAKLQLSKDTYDDDLDNLLAQIGDQ |
| | | YADLFLAAKNLSDAILLSDILRVNTEITKAPLSASMIKRYD |
| | | EHHQDLTLLKALVRQQLPEKYKEIFFDQSKNGYAGYIDG |
| | | GASQEEFYKFIKPILEKMDGTEELLVKLNREDLLRKQRTF |
| | | DNGSIPHQIHLGELHAILRRQEDFYPFLKDNREKIEKILTFR |
| | | IPYYVGPLARGNSRFAWMTRKSEETITPWNFEEVVDKGAS |
| | | AQSFIERMTNFDKNLPNEKVLPKHSLLYEYFTVYNELTKV |
| | | KYVTEGMRKPAFLSGEQKKAIVDLLFKTNRKVTVKQLKE |
| | | DYFKKIECFDSVEISGVEDRFNASLGTYHDLLKIIKDKDFL |
| | | DNEENEDILEDIVLTLTLFEDREMIEERLKTYAHLFDDKV |
| | | MKQLKRRRYTGWGRLSRKLINGIRDKQSGKTILDFLKSDG |
| | | FANRNFMQLIHDDSLTFKEDIQKAQVSGQGDSLHEHIANL |
| | | AGSPAIKKGILQTVKVVDELVKVMGRHKPENIVIEMAREN |
| | | QTTQKGQKNSRERMKRIEEGIKELGSQILKEHPVENTQLQ |
| | | NEKLYLYYLQNGRDMYVDQELDINRLSDYDVDHIVPQSF |
| | | LKDDSIDNKVLTRSDKNRGKSDNVPSEEVVKKMKNYWR |
| | | QLLNAKLITQRKFDNLTKAERGGLSELDKAGFIKRQLVET |
| | | RQITKHVAQILDSRMNTKYDENDKLIREVKVITLKSKLVS |
| | | DFRKDFQFYKVREINNYHHAHDAYLNAVVGTALIKKYPK |
| | | LESEFVYGDYKVYDVRKMIAKSEQEIGKATAKYFFYSNIM |
| | | NFFKTEITLANGEIRKRPLIETNGETGEIVWDKGRDFATVR |
| | | KVLSMPQVNIVKKTEVQTGGFSKESILPKRNSDKLIARKK |
| | | DWDPKKYGGFDSPTVAYSVLVVAKVEKGKSKKLKSVKE |
| | | LLGITIMERSSFEKNPIDFLEAKGYKEVKKDLIIKLPKYSLF |
| | | ELENGRKRMLASAGELQKGNELALPSKYVNFLYLASHYE |
| | | KLKGSPEDNEQKQLFVEQHKHYLDEIIEQISEFSKRVILAD |
| | | ANLDKVLSAYNKHRDKPIREQAENIIHLFTLTNLGAPAAF |
| | | KYFDTTIDRKRYTSTKEVLDATLIHQSITGLYETRIDLSQL |
| | | GGD (SEQ ID NO: 37) |
| No. | Proteins | Domains and amino acid positions |
| 1 | IscB(−HNH) | X domain: 51-97 |
| | EFH81386 | RuvC-I: 104-118 |
| | | Bridge Helix: 140-160 |
| | | RuvC-II: 169-212 |
| | | RuvC-III: 226-278 |
| 2 | IscB(+HNH) | X domain: 11-56 |
| | TAE54104.1 | RuvC-I: 63-77 |
| | | Bridge Helix: 100-121 |
| | | RuvC-II: 129-172 |
| | | HNH: 211-243 |
| | | RuvC-III: 279-321 |
| 3 | IscB(+HNH) | X domain: 4-50 |
| | WP_038093640.1 | RuvC-I: 57-71 |
| | | Bridge Helix: 108-129 |
| | | RuvC-II: 138-181 |
| | | HNH: 220-252 |
| | | RuvC-III: 288-330 |
| 4 | IscB(+HNH) | X domain: 7-52 |
| | WP_052490348.1 | RuvC-I: 59-73 |
| | | Bridge Helix: 100-121 |
| | | RuvC-II: 129-172 |
| | | HNH: 211-243 |
| | | RuvC-III: 279-322 |
| 5 | IscB(+HNH) | X domain: 7-52 |
| | WP_015325818.1 | RuvC-I: 61-75 |
| | | Bridge Helix: 101-121 |
| | | RuvC-II: 132-175 |
| | | HNH: 215-247 |
| | | RuvC-III: 284-327 |
| 6 | sp\|G3ECR1\| | RuvC-I: 28-42 |
| | CAS9_STRTR | Bridge Helix: 85-108 |
| | | Rec: 118-736 |
| | | RuvC-II: 750-799 |
| | | HNH: 864-896 |
| | | RuvC-III: 957-1019 |
| | | PAM Interaction (PI): 1119-1409 |
| 7 | sp\|J7RUA5\| | RuvC-I: 7-21 |
| | CAS9_STAAU | Bridge Helix: 49-72 |
| | | Rec: 80-433 |
| | | RuvC-II: 445-493 |
| | | HNH: 553-585 |
| | | RuvC-III: 654-709 |
| | | PAM Interaction (PI): 789-1053 |
| 8 | Streptococcus_ | RuvC-I: 4-18 |
| | pyogenes_SF370 | Bridge Helix: 61-84 |
| | | Rec: 94-718 |
| | | RuvC-II: 725-774 |
| | | HNH: 833-865 |
| | | RuvC-III: 926-988 |
| | | PAM Interaction (PI): 1099-1365 |

### X Domains

In some embodiments, the IscB proteins comprise an X domain, e.g., at its N-terminal.

In certain embodiments, the X domain include the X domains in Table 4. Examples of the X domains also include any polypeptides a structural similarity and/or sequence similarity to a X domain described in the art. In some examples, the X domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with X domains in Table 4.

In some examples, the X domain may be no more than 10, no more than 20, no more than 30, no more than 40, no more than 50, no more than 60, no more than 70, no more than 80, no more than 90, or no more than 100 amino acids in length. For example, the X domain may be no more than 50 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Y Domain

In some embodiments, the IscB proteins comprise a Y domain, e.g., at its C-terminal.

In certain embodiments, the X domain include Y domains in Table 4. Examples of the Y domain also include any polypeptides a structural similarity and/or sequence similarity to a Y domain described in the art. In some examples, the Y domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with Y domains in Table 4.

### RuvC Domain

In some embodiments, the IscB proteins comprises at least one nuclease domain. In certain embodiments, the IscB proteins comprise at least two nuclease domains. In certain embodiments, the one or more nuclease domains are only active upon presence of a cofactor. In certain embodiments, the cofactor is Magnesium (Mg). In embodiments where more than one nuclease domain is present and the substrate is a double-strand polynucleotide, the nuclease domains each cleave a different strand of the double-strand polynucleotide. In certain embodiments, the nuclease domain is a RuvC domain.

The IscB proteins may comprise a RuvC domain. The RuvC domain may comprise multiple subdomains, e.g., RuvC-I, RuvC-II and RuvC-III. The subdomains may be separated by interval sequences on the amino acid sequence of the protein.

In certain embodiments, examples of the RuvC domain include those in Table 4. Examples of the RuvC domain also include any polypeptides a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains in Table 4.

### Bridge Helix

The IscB proteins comprise a bridge helix (BH) domain. The bridge helix domain refers to a helix and arginine rich polypeptide. The bridge helix domain may be located next to anyone of the amino acid domains in the nucleic-acid guided nuclease. In some embodiments, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In certain embodiments, examples of the BH domain include those in Table 4. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Table 4.

### HNH Domain

The IscB proteins comprise an HNH domain. In certain embodiments, at least one nuclease domain shares a substantial structural similarity or sequence similarity to a HNH domain described in the art.

In some examples, the nucleic acid-guided nuclease comprises a HNH domain and a RuvC domain. In the cases where the RuvC domain comprises RuvC-I, RuvC-II, and RuvC-III domain, the HNH domain may be located between the Ruv C II and RuvC III subdomains of the RuvC domain.

In certain embodiments, examples of the HNH domain include those in Table 4. Examples of the HNH domain also include any polypeptides a structural similarity and/or sequence similarity to a HNH domain described in the art. For example, the HNH domain may share a structural similarity and/or sequence similarity to a HNH domain of Cas9. In some examples, the HNH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with HNH domains in Table 4.

hRNA

In some examples, the IscB proteins capable of forming a complex with one or more hRNA molecules. The hRNA complex can comprise a guide sequence and a scaffold that interacts with the IscB polypeptide. An hRNA molecules may form a complex with an IscB polypeptide nuclease or IscB polypeptide and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g., spacer, that is not derived from the same species as the IscB polypeptide nuclease, e.g. IscB protein. For example, a heterologous hRNA molecule of a IscB polypeptide nuclease derived from species A comprises a polynucleotide derived from a species different from species A, or an artificial polynucleotide.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35) z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein, the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 38)
MDPIRSRTPSPARELLSGPQPDGVQPTADRGVSPPAGGPLDGLP
ARRTMSRTRLPSPPAPSPAFSADSFSDLLRQFDPSLFNTSLFDS
LPPFGAHHTEAATGEWDEVQSGLRAADAPPPTMRVAVTAARP
PRAKPAPRRRAAQPSDASPAAQVDLRTLGYSQQQQEKIKPKVR
STVAQHHEALVGHGFTHAHIVALSQHPAALGTVAVKYQDMIA
ALPEATHEAIVGVGKQWSGARALEALLTVAGELRGPPLQLDT
GQLLKIAKRGGVTAVEAVHAWRNALTGAPLN.

An exemplary amino acid sequence of a C-terminal capping region is:

(SEQ ID NO: 39)
[0230]RPALESIVAQLSRPDPALAALTNDHLVALACLGGRPALDA
VKKGLPHAPALIKRTNRRIPERTSHRVADHAQVVRVLGFFQCH
SHPAQAFDDAMTQFGMSRHGLLQLFRRVGVTELEARSGTLPP
ASQRWDRILQASGMKRAKPSPTSTQTPDQASLHAFADSLERDL
DAPSPMHEGDQTRAS.

As used herein, the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933, 113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein, “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Polypeptides

In certain example embodiments, the cargo molecule may one or more polypeptides. The polypeptide may be a full-length protein or a functional fragment or functional domain thereof, that is a fragment or domain that maintains the desired functionality of the full-length protein. As used within this section, “protein” is meant to refer to full-length proteins and functional fragments and domains thereof. A wide array of polypeptides may be delivered using the engineered delivery vesicles described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered delivery vesicles and systems described herein via delivery of the corresponding encoding polynucleotide.

### Secretory Proteins

In certain example embodiments, the one or more polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In some embodiments, the protein is secreted by the secretory pathway. In some embodiments, the proteins are exocrine secretion proteins or peptides, comprising enzymes in the digestive tract. In some embodiments the protein is endocrine secretion protein or peptide, for example, insulin and other hormones released into the blood stream. In some embodiments, the protein is involved in signaling between or within cells via secreted signaling molecules, for example, paracrine, autocrine, endocrine or neuroendocrine. In some embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., al-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a specific embodiment, the one or more polynucleotides is inserted in the middle of the c-peptide. In some aspects, the secretory protein is GLP-1, glucagon, betatrophin, pancreatic amylase, pancreatic lipase, carboxypeptidase, secretin, CCK, a PPAR (e.g. PPAR-alpha, PPAR-gamma, PPAR-delta or a precursor thereof (e.g. preprotein or preproprotein). In aspects, the secretory protein is fibronectin, a clotting factor protein (e.g. Factor VII, VIII, IX, etc.), a2-macroglobulin, al-antitrypsin, antithrombin III, protein S, protein C, plasminogen, a2-antiplasmin, complement components (e.g. complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cells in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+thymocytes, CD4−/CD8-thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th 17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins may be immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-β, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-2, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR), or may include one or more PPARs. In some embodiments, the protein is PPARα, PPAR γ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and/or Transplant Survival Functions

In certain example embodiments, the one or more polypeptides may comprise an proteins that proteins that promote tissue regeneration and/or transplant survival functions. In some cases, such proteins may induce and/or up-regulate the expression of genes for pancreatic β cell regeneration. In some cases, the proteins that promote transplant survival and functions include the products of genes for pancreatic β cell regeneration. Such genes may include proislet peptides that are proteins or peptides derived from such proteins that stimulate islet cell neogenesis. Examples of genes for pancreatic β cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfa1, Mafa, Pax6, Pax4, Nkx6.1, Nkx2.2, PDGF, vglycin, placental lactogens (somatomammotropins, e.g. CSH1, CHS2), isoforms thereof, homologs thereof, and orthologs thereof. In certain embodiments, the protein promoting pancreatic B cell regeneration is a cytokine, myokine, and/or adipokine.

### Hormones

In certain embodiments, the one or more polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoidds. In a particular embodiment, the hormone is insulin or incretins such as exenatide, GLP-1.

### Neurohormones

In embodiments, the secreted peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is α-defensin HD-6, HNP-1 and β-defensin hBD-3, lysozyme, cathelcidin LL-37, C-type lectin RegIIIalpha, for example. See, e.g. Wang, “Human Antimicrobial Peptide and Proteins” Pharma, May 2014, 7 (5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some aspects the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed with. In some aspects, the anti-fibrillating agent is P4 (VITYF) (SEQ ID NO: 40), P5 (VVVVV) (SEQ ID NO: 41), KR7 (KPWWPRR) (SEQ ID NO: 42), NK9 (NIVNVSLVK) (SEQ ID NO: 43), iAb5p (Leu-Pro-Phe-Phe-Asp) (SEQ ID NO: 44), KLVF (SEQ ID NO: 45) and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3: e01681, D-(PGKLVYA) (SEQ ID NO: 49), RI-OR2-TAT, cyclo(17, 21)-(Lys17, Asp21) A_(1-28), SEN304, SEN1576, D3, R8-AB (25-35), human yD-crystallin (HGD), poly-lysine, heparin, poly-Asp, polyGl, poly-L-lysine, poly-L-glutamic acid, LVEALYL (SEQ ID NO: 46), RGFFYT (SEQ ID NO: 47), a peptide set forth or as designed/generated by the method set forth in U.S. Pat. No. 8,754,034, and combinations thereof. In some embodiments, the anti-fibrillating agent is a D-peptide. In some embodiments, the anti-fibrillating agent is an L-peptide. In some embodiments, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In some embodiments, the retro-inverso modified peptide is derived from a natural or synthetic Aβ peptide. In some aspects, the polynucleotide encodes a fibrillation resistant protein. In some aspects, the fibrillation resistant protein is a modified insulin, see e.g. U.S. Pat. No. 8,343,914.

### Antibodies

In certain embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such, these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, lgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, lgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-y4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×107 M−1 (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, InM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CHI domains; (ii) the Fab′ fragment, which is a Fab fragment having one or more cysteine residues at the C-terminus of the CHI domain; (iii) the Fd fragment having VH and CHI domains; (iv) the Fd′ fragment having VH and CHI domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8 (10): 1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92 (6): 1981-1988 (1998); Chen et al., Cancer Res. 58 (16): 3668-3678 (1998); Harrop et al., J. Immunol. 161 (4): 1786-1794 (1998); Zhu et al., Cancer Res. 58 (15): 3209-3214 (1998); Yoon et al., J. Immunol. 160 (7): 3170-3179 (1998); Prat et al., J. Cell. Sci. III (Pt2): 237-247 (1998); Pitard et al., J. Immunol. Methods 205 (2): 177-190 (1997); Liautard et al., Cytokine 9 (4): 233-241 (1997); Carlson et al., J. Biol. Chem. 272 (17): 11295-11301 (1997); Taryman et al., Neuron 14 (4): 755-762 (1995); Muller et al., Structure 6 (9): 1153-1167 (1998); Bartunek et al., Cytokine 8 (1): 14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Protease Cleavage Sites

The one or more cargo polypeptides, as exemplified above, may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein. Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1 (ULP-1) protease cleavage site. In certain examples, the protease cleavage site comprises Lys-Arg.

### Small Molecules

In some embodiments, the engineered delivery vesicle can deliver one or more small molecule compounds. Thus, in some embodiments, the cargo molecule is a small molecule. In some embodiments, the small molecule compound(s) can be linked or directly attached to a polynucleotide that can bind a polynucleotide binding protein that can be included in the engineered delivery system polynucleotide. In some embodiments, the engineered delivery system polynucleotide can include a small molecule binding protein (e.g. a receptor for the small molecule) that, like the polynucleotide binding protein discussed elsewhere herein, can be incorporated in to the engineered delivery vesicle.

In some embodiments, the small molecule compound(s) can be linked or directly attached to a polynucleotide that can bind a polynucleotide binding protein that can be included in the engineered delivery system polynucleotide or delivery vesicle. In some embodiments, the engineered delivery system polynucleotide or delivery vesicle can include a small molecule binding protein (e.g. a receptor for the small molecule) that, like the polynucleotide binding protein discussed elsewhere herein, can be incorporated in to the engineered delivery system polynucleotide or delivery vesicle.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosteron Cortisol). Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### Engineered Cells

Described herein are various aspects of engineered cells that can include one or more of the engineered delivery system polynucleotides, polypeptides, vectors, and/or vector systems, and/or engineered delivery vesicles (e.g., those produced from an engineered delivery system polynucleotide and/or vector(s)) described elsewhere herein. In some aspects, the engineered cells can express one or more of the engineered delivery system polynucleotides and/or can produce one or more engineered delivery vesicles, which are described in greater detail herein. Such cells are also referred to herein as “producer cells” or donor cells, depending on the context. It will be appreciated that these engineered cells are different from “modified cells” described elsewhere herein in that the modified cells are not necessarily producer or donor cells (e.g., they do not make engineered delivery vesicles) unless they include one or more of the engineered delivery system molecules or vectors described herein that render the cells capable of producing an engineered delivery vesicle. Modified cells can be recipient cells of an engineered delivery vesicle and can, in some embodiments, be said to be modified by the engineered delivery vesicles and/or a cargo present in the engineered delivery vesicle that is delivered to the recipient cell. The term “modification” can be used in connection with modification of a cell that is not dependent on being a recipient cell. For example, isolated cells can be modified prior to receiving an engineered delivery system or engineered delivery vesicle and/or cargo.

In an aspect, the invention provides a non-human eukaryotic organism; for example, a multicellular eukaryotic organism, including a eukaryotic host cell containing one or more components of an engineered delivery system described herein according to any of the described embodiments. In other aspects, the invention provides a eukaryotic organism; preferably a multicellular eukaryotic organism, comprising a eukaryotic host cell containing one or more components of an engineered delivery system described herein according to any of the described embodiments. In some embodiments, the organism is a host of lentivirus or AAV.

The engineered cell can be any eukaryotic cell, including but not limited to, human, non-human animal, plant, algae, and the like.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces. Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21 (DE3), DL21 (DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue (DE3), BLR, C41 (DE3), C43 (DE3), Lemo21 (DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some aspects the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CVI, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML TI, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

Further, the engineered cell may be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered delivery vesicle is produced it can package one or more molecules that are within the producer cell that can be related to the desired physiological and/or biological characteristic. In this context, the cargo molecules incorporated into the delivery vesicles can be capable of transferring the desired characteristic to a recipient cell.

In some embodiments, a cell can be obtained from a subject, modified such that it is an engineered delivery vesicle producer cell, and administered back to the subject from which it was obtained (autologous) or delivered to an allogenic subject. In other words, a producer cell described herein can be used in an autologous or allogenic context, such as in a cell therapy. In these embodiments, the cells can deliver a cargo, such as a therapeutic cargo or a cargo that can manipulate a cellular microenvironment within the subject.

Conventional viral and non-viral based gene transfer methods can be used to introduce nucleic acids (e.g. such as one or more of the polynucleotides of the engineered delivery system described herein) in cells or target tissues. Such methods can be used to administer nucleic acids encoding components of a nucleic acid-targeting system to cells in culture, or in a host organism. In some aspects, a delivery is via a polynucleotide molecule (e.g. a DNA or RNA molecule) not contained in a vector. In some aspects, delivery is via a vector. In some aspects, delivery, is via viral particles. In some aspects, delivery is via a particle, (e.g. a nanoparticle) carrying one or more engineered delivery system polynucleotides, vectors, or viral particles. Particles, including nanoparticles, are discussed in greater detail elsewhere herein.

Vector delivery can be appropriate in some aspects, where in vivo expression is envisaged. It will be appreciated that the engineered cells can be generated in vitro, ex vivo, in situ, or in vivo by delivery of one or more components of the engineered delivery systems as described elsewhere herein.

Suitable conventional viral and non-viral based methods of engineering cells to contain and/or express the engineered delivery system polynucleotides and/or vectors described herein are generally known in the art and/or described elsewhere herein.

### Formulations

Component(s) of the engineered delivery system, engineered cells, engineered delivery vesicles, or combinations thereof can be included in a formulation that can be delivered to a subject or cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, and combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In aspects where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In aspects where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

### Pharmaceutically Acceptable Carriers and Auxiliary Ingredients and Agents

In aspects, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered delivery vesicles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g. micronized) compound or salt or solvate thereof, is defined by a Dso value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g. the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, manitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. Such unit doses may therefore be administered once or more than once a day. Such pharmaceutical formulations may be prepared by any of the methods well known in the art.

### EXAMPLES

### Example 1: Pseudotyped Lentiviral Vectors

The host range of retroviral vectors including lentiviral vectors can be expanded or altered by a process known as pseudotyping. Pseudotyped lentiviral vectors consist of vector particles bearing glycoproteins (GPs) derived from other enveloped viruses. Such particles possess the tropism of the virus from which the GP was derived. For example, to exploit the natural neural tropism of rabies virus, vectors designed to target the central nervous system have been pseudotyped using rabies virus-derived GPs. Among the first and still most widely used GPs for pseudotyping lentiviral vectors is the vesicular stomatitis virus GP (VSV-G), due to the very broad tropism and stability of the resulting pseudotypes. Pseudotypes involving VSV-G have become effectively the standard for evaluating the efficiency of other pseudotypes.

A series of lentiviral pseudotypes were produced using lentiviral vectors (an example set of vectors is shown in FIG. 1A) wherein each pseudotype had a different envelope protein (FIG. 1B). The viruses that were produced were screened for successful production (ability to form viral particles) and function (i.e., ability to infect target cells). It was observed that different pseudotypes produce at different rates. Finctional evaluation of the viral psudotypes was performed on a panel of cell lines that included HEK293T cells, A549+Ace2 cells (Ace2-expressing A549 cells), HepG1 cells, OUMS23 cells and Jurkat cells. Tropism on functional pseudotypes was not highly specific. It also was not clear if some pseudotypes even produced any functional particles. Pseudotypes that show functionality on any of the tested cell lines are shown in Table 1.

TABLE 1
Pseudotypes that show functionality on any one of the
tested cell lines.

| Number | ID | Family - Name |
|---|---|---|
| 1 | LASV_GPC | Arenaviridae Lassa mammarenavirus |
| 2 | LCMV_GPC | Arenaviridae Lymphocytic Choriomeningitis |
| | | virus |
| 3 | BoDV1_G | Bornaviridae Mammalian Bornavirus 1 |
| 4 | EBOV_GP | Filoviridae Ebola virus |
| 5 | MARV_GP | Filoviridae Marburg virus |
| 6 | DHOV_GP | Orthomyxoviridae Dhori virus |
| 7 | FIV_Env | Retroviridae Feline immunodeficiency virus |
| 8 | DUVV_G | Rhabdoviridae Duvenhage lyssavirus |
| 9 | EBLV-1_G | Rhabdoviridae European bat 1 lyssavirus |
| 10 | ISFV_G | Rhabdoviridae Isfahan virus |
| 11 | MOKV_G | Rhabdoviridae Mokola virus |
| 12 | RV_G | Rhabdoviridae Rabies virus |
| 13 | VSV_G | Rhabdoviridae Arizona vesiculovirus |
| 14 | CHIKV_E1E2 | Togaviridae Chikungunya virus |
| 15 | EEE_E1E2 | Togaviridae Eastern equine encephalitis virus |
| 16 | ONNV_E1E2 | Togaviridae O'nyong'nyong virus |
| 17 | SFV_E1E2 | Togaviridae Semliki Forest virus |
| 18 | HTNV_GP | Hantaviridae Hantaan orthohantavirus |
| 19 | DUGV_GP | Bunyaviridae Dugbe virus |
| 20 | MD2G | Rhabdoviridae Indiana vesiculovirus |
| 21 | LACV_GP | Bunyaviridae La Crosse virus |
| 22 | SARS-COV- | Coronaviridae Severe acute respiratory |
| | 2_Sdel18 | syndrome coronavirus 2 |
| 23 | FLUAV_HA | Orthomyxoviridae Influenza A virus |
| 24 | m168 | Togaviridae Sindbis virus |
| 25 | Bless | Retroviridae Baboon endogenous virus |

### Example 2: Engineering of Sindbis Envelope (m168) Targeting Using Antibodies

As a proof of concept, the inventors engineered the Sindbis virus envelope protein (m168) by using antibodies for retargeting. Protein A was fused to the Sindbis virus envelope protein (FIG. 2A), and pseudotyped lentiviruses expressing this fusion envelope protein were produced. Ace2-expressing and control A549 cells were incubated with the virus and anti-Ace2 antibody. Ace2-expressing cells were specifically targeted by viruses that expressed the protein A-fused Sindbis virus envelope protein. See FIG. 2B.

In another experiment, Protein A on the Sindbis virus envelope protein was replaced with a SNAP tag sequence (FIG. 2C) which can bind to an antibody labeled with benzylguanine using click chemistry (FIG. 2D). Pseudoviruses that were covalently liked to an antibody using a SNAP tag had much better targeting to specific cells compared to control. See FIGS. 2E and 2F.

Based on these successful proof-of-concept experiments, the inventors devised specific, modular and versatile targeted delivery vehicle where the targeting and fusion functions are separated (FIG. 2G).

The inventors found that only a small amount of targeting molecule is sufficient for targeted transduction. Viruses that expressed different ratios (1:2, 1:5 and 1:10) of Targeting molecule/Fusogen were compared. Consistently, viruses that expressed Targeting molecule/Fusogen at 1:5 or 1:10 transduced at higher levels. See FIGS. 2H and 21. High levels of targeting moieties may result in lower transduction, possibly due to reduction in fusogen expression by promoter competition or due to interference with fusogen trimerization.

Next, the envelope protein was expressed from a different promoter than other viral particles. It was found that expression of the helper envelope from a different promoter increased production of viral particles. See, FIG. 2J-2K.

Overall, antibody-based retargeting of Sindbis envelope allowed specific targeted transduction of target cells. See FIGS. 2L-2N.

### Example 3: The Antibody-Based Retargeting Approach Works in Different Pseudotypes

Inventors of this disclosure tested multiple fusogens with antibody-based retargeting and determined suitable pseudotypes (see Table 2).

TABLE 2
Pseudotypes suitable for antibody-based retargeting

| Number | ID | Family - Name |
|---|---|---|
| 1 | PICV_GPC | Arenaviridae Pichinde virus |
| 2 | EBOV_GP | Filoviridae Ebola virus |
| 3 | DHOV_GP | Rhabdoviridae Duvenhage lyssavirus |
| 4 | DUVV_G | Rhabdoviridae European bat 1 lyssavirus |
| 5 | MARV_GP | Filoviridae Marburg virus |
| 6 | DHOV_GP | Orthomyxoviridae Dhori virus |
| 7 | EBLV-1_G | Rhabdoviridae European bat 1 lyssavirus |
| 8 | ISFV_G | Rhabdoviridae Isfahan virus |
| 9 | CHIKV_E1E2 | Togaviridae Chikungunya virus |
| 10 | EEE_E1E2 | Togaviridae Eastern equine encephalitis virus |
| 11 | ONNV_E1E2 | Togaviridae O'nyong'nyong virus |
| 12 | RuV_E1E2 | Matonaviridae Rubella virus |
| 13 | HTNV_GP | Hantaviridae Hantaan orthohantavirus |
| 14 | DUGV_GP | Bunyaviridae Dugbe virus |
| 15 | LACV_GP | Bunyaviridae La Crosse virus |
| 16 | FLUAV_HA | Orthomyxoviridae Influenza A virus |
| 17 | QRFV_Hyp | Orthomyxoviridae Quaranfil virus |
| 18 | m168 | Togaviridae Sindbis virus |

Retargeting the Chikungunya virus envelope protein. Wild type Chikungunya virus envelope protein (CHIKV) normally shows low transduction efficiency for HEK293FT cells. CHIKV was fused to a Protein AG moiety (called “CHIKV+pAG”). The pseudotyped virus containing CHIKV+pAG was able to efficiently and specifically target and transduce HEK293FT cells in the presence of MHCI antibodies (αMHCI) that can bind these cells. See FIG. 3A.

Retargeting the Cocal virus envelope protein. The cocal virus envelope protein (COCV) is related to Vesicular Stomatitis Virus Envelope protein (VSV-G), but it is not inactivated by serum and complement proteins. The inventors found that COCV can also be retargeted using an antibody-based approach. Briefly, COCV was fused to protein AG and incubated with target HEL 293 FT cells in the presence or absence of MHCI antibodies. It was observed that pseudotyped viruses that displayed COCV+pAG specifically and efficiently targeted HEK293FT cells in the presence of MHCI antibodies. FIG. 3B.

### Example 4: Supplementing or Abolishing the Intrinsic Tropisms of Envelope Proteins

Wild type viral envelope proteins show differing degrees of tropism towards different cell types. See FIGS. 3C and 3D. With the methods of the instant disclosure, it is possible to supplement or replace the natural tropism of an envelope protein. For instance, wild-type VSV-G targets cells with LDL receptor (LDLR). One can add additional targeting moieties (e.g., antibodies) and add more targets to the VSV-G target repertoire or one can abolish the intrinsic tropism of an envelope protein (through point mutations or deletions in the targeting domain of the envelope protein) and have the added targeting moiety (e.g., antibody) alone determine the tropism.

The inventors aimed to abolish any intrinsic tropism of viral envelopes so that the targeting can be engineered towards a cell type of interest and any off-target effects can be minimized.

VSV-G. The inventors introduced the following point mutations to VSV-G: H8A, K47Q, Y209A and R354Q and observed that point mutations decrease virus infectivity by affecting titer and transduction efficiency. See FIGS. 3E-3F. The inventors identified K47Q and R354Q as the most potent mutations in disrupting VSV-G inherent tropism.

The inventors then constructed double mutants of VSV-G and found that double mutants decrease infection even further. See FIGS. 3G-3H. The inventors identified K47Q-R354Q double mutant as having only minimal residual targeting activity.

A panel of adherent cell lines expressing ClassI were tested for transduction with the VSV-G K47Q-R354Q double mutant. Some cell lines showed high basal transduction (A172, HUH7). See FIG. 3I. Presence of anti-classI antibody (aClassI) boosted infection rates up to 30-fold. See FIG. 3I. 50000 Jurkat T cells were infected with the indicated amounts of concentrated virus (y-axis) that was pre-incubated with the indicated amounts of antibody (x-axis) (FIG. 3J). In absence of antibody, there was very low transduction (white squares in FIG. 3J).

Jurkat-Surf-GFP cells can be transduced with αGFP targeted virus: VSV-G K47Q-R354Q double mutant virus were targeted to Jurkat+surfGFP cells (Jurkat cells expressing GFP on their surface) with protein AG (pAG) and anti-GFP antibodies. Briefly, Jurkat+surfGFP were transduced with indicated amounts of concentrated virus (1000×) (FIG. 3K). Cells were stained with αGFP antibody (homebrew) and subsequently incubated with indicated virus amounts (FIG. 3K). The cells were analyzed by flow cytometry after 5 days. See FIG. 3K.

Cocal virus envelope. The following amino acids were identified as important for inherent tropism of cocal virus envelope: Q25, K64, Y226, and R371 (the numbering refers to the full length sequence before processing (i.e., including the secretion signal)).

Chikungunya virus envelope. The following amino acids were identified as important for inherent tropism of Chikungunya virus envelope: W64, D71, T116 and 1121 in A domain in E2, and 1190, Y199 and 1217 in B domain in E2. The most pronounced infectivity decreases were observed for conserved residues 1190, Y199 and 1217. See FIGS. 3L-3N.

HEK293FT Surf-GFP transduced with αGFP-targeted viruses. HEK293FT+surfGFP (HEK293FT cells expressing GFP on their surfaces) transduced with indicated amounts of concentrated virus with retargeted Chikungunya virus envelope (WT and I217A mutant) (100×). All conditions in presence of αGFP antibody. Analyzed for high and low GFP on target cells. HEK293FT cells FIG. 3O.

### Example 5: Alternative Strategies for Targeting

The inventors next explored alternative targeting moieties such as scFvs (FIG. 4A).

Viruses expressing Chikungunya virus envelope E1E2 (CHIKV-E1E2) fused to scFV against HA (anti-HA scFV) was developed. HEK 293 FT+Surf-HA cells (HEK293FT cells expressing HA on the surface) were targeted. HEK 293 FT were control nontarget cells. Anti-HA scFv was able to efficiently and selectively target HEK 293 FT+Surf-HA cells. See FIG. 4B.

### Alternative Envelopes

In addition to a pseudotyped lentivirus, the inventors envision that other vesicle-based delivery vehicles can be utilized for delivery methods of this disclosure such as selective endogenous encapsidation for cellular delivery system (SEND), nanoblade, an engineered virus-like particle (eVLP), or gesicle.

Directed viral envelope works with SEND. Using selective endogenous encapsidation for cellular delivery system (SEND) as the envelope structure, a directed cocal envelope protein was used to target cells. Briefly, 5000 A549+Ace2+CreReporter cells were incubated with 30 μl virus+1 μl αAce2 antibody. Cells were analyzed by Flow Cytometry after 3 days. See FIG. 4C.

Different Fusion Proteins can be retargeted. Fusion proteins can be classified in three groups, as Class I-III fusogens (FIG. 4D), and any fusogen from any class can be retargeted using the approaches disclose herein.

Class I fusogens show structural and sequence similarity in envelope sequences. For instance, Baculoviral envelope GP64 shows high structural similarity with Orthomyxoviral envelopes. Quaranfil Quaranjavirus (QRFVHA) shares 55% sequence similarity with GP64, Dhori Virus (DHOVGP) shares 60% sequence similarity with GP64. Class I representatives QRFV-HA and DHOVGP work with antibody retargeting. GP64 can also be retargeted and withstands freeze-thaw. See FIG. 4E. Different amounts of unconcentrated virus were used on 10000 HEK293FT cells. Increased infection in presence of antibody (˜5-fold increase) was observed (FIG. 4E). Basal infection of cells is believed to be caused due to cholesterol binding on target cells. MYENDLL (SEQ ID NO: 50) is a Cholesterol Recognition Amino Acid Consensus (CRAC) motif. Y311A mutation (in MYENDLL (SEQ ID NO: 50)) abolishes Cholesterol binding and can be used to reduce the basal infection rate.

Some Class II fusion proteins are pH-dependent (e.g. Alphaviruses use an E1E2 dimer as fusogen—examples include Sindbis Virus E2, VSV-G, Cocal Virus G and Chikungunya virus envelope E1E2). These fusion proteins go through attachment/receptor binding, clathrin-mediated endocytosis, endosomal acidification which induces membrane fusion (when pH drops) and release of the cargo. See, FIG. 4F. Receptor binding is mediated by E2 and fusion is initiated by E1 upon exposure to low pH. E1 shows high structure and sequence conservation among different viruses.

Some Class III fusion proteins are also pH-dependent (e.g., Rhabdoviral G proteins). These fusogens have a conserved two partite fusion loop, which mediates fusion upon exposure to low pH. The pH sensor on these fusogens comprises a conserved histidine residue.

### Sequences

SEQ ID NO: 1 (VSV-G Nucleotide Sequence):
atgaagtgccttttgtacttagcctttttattcattggggtgaattgcaagttcaccatagtttttccacacaaccaaaaaggaaactggaaaa
atgttccttctaattaccattattgcccgtcaagctcagatttaaattggcataatgacttaataggcacagccttacaagtcaaaatgcccaa
gagtcacaaggctattcaagcagacggttggatgtgtcatgcttccaaatgggtcactacttgtgatttccgctggtatggaccgaagtatata
acacattccatccgatccttcactccatctgtagaacaatgcaaggaaagcattgaacaaacgaaacaaggaacttggctgaatccaggcttcc
ctcctcaaagttgtggatatgcaactgtgacggatgccgaagcagtgattgtccaggtgactcctcaccatgtgctggttgatgaatacacagg
agaatgggttgattcacagttcatcaacggaaaatgcagcaattacatatgccccactgtccataactctacaacctggcattctgactataag
gtcaaagggctatgtgattctaacctcatttccatggacatcaccttcttctcagaggacggagagctatcatccctgggaaaggagggcacag
ggttcagaagtaactactttgcttatgaaactggaggcaaggcctgcaaaatgcaatactgcaagcattggggagtcagactcccatcaggtgt
ctggttcgagatggctgataaggatctctttgctgcagccagattccctgaatgcccagaagggtcaagtatctctgctccatctcagacctca
gtggatgtaagtctaattcaggacgttgagaggatcttggattattccctctgccaagaaacctggagcaaaatcagagcgggtcttccaatct
ctccagtggatctcagctatcttgctcctaaaaacccaggaaccggtcctgctttcaccataatcaatggtaccctaaaatactttgagaccag
atacatcagagtcgatattgctgctccaatcctctcaagaatggtcggaatgatcagtggaactaccacagaaagggaactgtgggatgactgg
gcaccatatgaagacgtggaaattggacccaatggagttctgaggaccagttcaggatataagtttcctttatacatgattggacatggtatgt
tggactccgatcttcatcttagctcaaaggctcaggtgttcgaacatcctcacattcaagacgctgcttcgcaacttcctgatgatgagagttt
attttttggtgatactgggctatccaaaaatccaatcgagcttgtagaaggttggttcagtagttggaaaagctctattgcctcttttttcttt
atcatagggttaatcattggactattcttggttctccgagttggtatccatctttgcattaaattaaagcacaccaagaaaagacagatttata
cagacatagagatgaaccgacttggaaagtaa
SEQ ID NO: 2 (VSV-G Amino acid Sequence):
MKCLLYLAFLFIGVNCKFTIVFPHNQKGNWKNVPSNYHYCPSSSDLNWHNDLIGTALQ
VKMPKSHKAIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFINGKCSNYICP
TVHNSTTWHSDYKVKGLCDSNLISMDITFFSEDGELSSLGKEGTGFRSNYFAYETGGKA
CKMQYCKHWGVRLPSGVWFEMADKDLFAAARFPECPEGSSISAPSQTSVDVSLIQDVE
RILDYSLCQETWSKIRAGLPISPVDLSYLAPKNPGTGPAFTIINGTLKYFETRYIRVDIAAPI
LSRMVGMISGTTTERELWDDWAPYEDVEIGPNGVLRTSSGYKFPLYMIGHGMLDSDLH
LSSKAQVFEHPHIQDAASQLPDDESLFFGDTGLSKNPIELVEGWFSSWKSSIASFFFIIGLII
GLFLVLRVGIHLCIKLKHTKKRQIYTDIEMNRLGK*
SEQ ID NO: 3 (COCV-G Nucleotide Sequence):
ATGAATTTTCTGCTCCTTACCTTTATAGTGCTCCCACTCTGTAGTCACGCGAAATTTA
GTATTGTATTCCCCCAATCTCAAAAGGGCAATTGGAAGAACGTACCAAGCAGCTACC
ACTATTGCCCCAGTAGTTCCGACCAGAATTGGCATAATGACCTCTTGGGGATCACTA
TGAAGGTGAAGATGCCAGCGACGCACAAGGCTATCCAAGCCGACGGCTGGATGTGC
CATGCAGCGAAATGGATTACGACATGCGATTTTCGCTGGTACGGGCCAAAGTACAT
AACACACAGCATACATAGTATACAACCTACTTCCGAGCAATGCAAGGAGAGTATTA
AGCAAACTAAGCAGGGGACCTGGATGAGCCCGGGATTTCCGCCACAGAACTGCGGT
TATGCCACTGTGACGGATAGTGTAGCTGTCGTTGTCCAAGCAACCCCGCATCACGTC
CTCGTCGATGAGTACACGGGTGAATGGATTGATTCTCAATTCCCTAACGGAAAATGT
GAAACAGAGGAATGTGAGACAGTTCATAATTCAACGGTGTGGTATTCAGACTACAA
AGTTACAGGCTTGTGTGATGCGACGTTGGTCGACACAGAAATCACGTTCTTTAGTGA
AGATGGCAAAAAGGAATCCATCGGAAAACCAAACACAGGTTACCGCAGCAACGCA
TTTGCTTATGAAAAAGGGGACAAGGTCTGTAAAATGAATTATTGTAAACACGCGGG
TGTGAGACTCCCATCCGGCGTCTGGTTTGAaTTtGTGGACCAGGATGTCTATGCGGCG
GCTAAGCTTCCTGAGTGCCCAGTCGGTGCTACTATCAGTGCGCCCACACAGACATCA
GTTGATGTCAGCCTCATACTGGATGTCGAACGtATTCTGGATTACTCCCTTTGCCAAG
AGACATGGAGTAAGATCAGGTCCAAGCAGCCCGTAAGCCCGGTAGACCTGTCCTAT
CTCGCACCGAAGAACCCTGGTACTGGACCTGCTTTTACAATCATAAATGGAACCCTT
AAATATTTCGAAACTCGCTACATACGCATTGACATAGACAACCCGATAATCTCCAAA
ATGGTCGGAAAAATAAGTGGCTCACAAACGGAGGCCGAACTTTGGACAGAATGGTT
TCCTTATGAAGGGGTGGAGATCGGACCCAACGGAATTTTGAAGACACCAACAGGAT
ACAAGTTTCCACTGTTTATGATTGGACATGGCATGTTGGATTCTGACCTTCACAAAA
CGTCCCAAGCAGAAGTATTCGAGCACCCACATCTCGCCGAAGCGCCAAAGCAGCTT
CCTGAAGAGGAAACTCTTTTCTTTGGTGACACGGGGATTAGTAAGAATCCCGTAGAA
CTGATTGAGGGGTGGTTTAGTTCCTGGAAGTCAACAGTCGTAACCTTTTTTTTCGCAA
TAGGTGTGTTTATTTTGCTCTACGTGGTCGCCAGGATAGTTATCGCCGTCCGGTACCG
CTATCAAGGGTCTAATAATAAGCGGATATACAACGACATCGAGATGAGTCGATTTC
GCAAAtga
SEQ ID NO :4 (COCV-G Amino acid Sequence):
MNFLLLTFIVLPLCSHAKFSIVFPQSQKGNWKNVPSSYHYCPSSSDQNWHNDLLGITMK
VKMPKTHKAIQADGWMCHAAKWITTCDFRWYGPKYITHSIHSIQPTSEQCKESIKQTKQ
GTWMSPGFPPQNCGYATVTDSVAVVVQATPHHVLVDEYTGEWIDSQFPNGKCETEECE
TVHNSTVWYSDYKVTGLCDATLVDTEITFFSEDGKKESIGKPNTGYRSNYFAYEKGDKV
CKMNYCKHAGVRLPSGVWFEFVDQDVYAAAKLPECPVGATISAPTQTSVDVSLILDVE
RILDYSLCQETWSKIRSKQPVSPVDLSYLAPKNPGTGPAFTIINGTLKYFETRYIRIDIDNPI
ISKMVGKISGSQTERELWTEWFPYEGVEIGPNGILKTPTGYKFPLFMIGHGMLDSDLHKT
SQAEVFEHPHLAEAPKQLPEEETLFFGDTGISKNPVELIEGWFSSWKSTVVTFFFAIGVFIL
LYVVARIVIAVRYRYQGSNNKRIYNDIEMSRFRK*
SEQ ID NO: 5 (CHIKV-E1E2 Nucleotide Sequence):
atggagtttatccctacacaaacattttataacagaaggtatcagcctagaccttggacccccagacctacaattcaagtgattagacctaggc
ctagacctcaaaggcaagccggacagctcgcccagctcatctccgctgtcaacaaactgaccatgagggccgtcccccaacagaagcct
agaaaaaacagaaagaacaaaaaacagaaacagaagcagcaagccccccagaataacacaaatcagaagaagcagccccctaaaaag
aagcccgcccagaagaagaagaagcccggaagaagagagagaatgtgtatgaaaatcgaaaatgactgcattttcgaggtcaaacacga
aggcaaggtgaccggctacgcttgtctggtgggcgacaaggtgatgaagcccgcccacgtgaagggcacaatcgacaacgccgatctg
gccaagctggcctttaagaggtccagcaagtacgacctcgaatgtgcccagatccccgtgcacatgaagtccgatgccagcaagttcacac
acgagaagcccgaaggctactataactggcaccatggagccgtgcagtatagcggaggaagatttaccattcccaccggagccggaaaa
cccggcgacagcggaaggcccattttcgacaacaagggcagagtggtcgccatcgtgctcggaggcgctaacgaaggagctagaacag
ctctctccgtggtgacatggaacaaagacatcgtcaccaagatcacacccgagggagctgaggaatggagcctcgccattcccgtgatgtg
tctgctggctaacacaacattcccttgcagccagcctccttgcattccttgttgctacgagaaggagcccgaagagacactgaggatgctcg
aggataacgtcatgagacccggctactatcagctgctgcaagcctctctgacatgctccccccataggcagagaagatccaccaaggataa
ctttaacgtgtataaggccacaagaccctacctcgctcattgccccgactgcggcgagggccatagctgtcacagccccgtggctctggaa
aggattaggaacgaggctaccgatggaacactgaagatccaagtctctctgcagatcggcattggcacagacgattcccacgattggacaa
agctgagatacatggacaatcacatccccgccgatgccggaagagccggactctttgtgaggacaagcgctccttgcaccatcaccggca
caatgggccattttatcctcgccagatgtcccaaaggcgagacactgacagtcggcttcaccgattccagaaagattagccacagctgcacc
caccctttccatcatgacccccccgtgattggaagggagaagttccatagcagacctcaacacggaaaggaactcccttgctccacctatgt
gcaaagcaatgccgccaccgccgaagagatcgaggtgcatatgccccccgacacccccgacagaacactgctgagccaacaatccggc
aacgtgaagatcacagtcaacagccagaccgtgaggtacaagtgcaactgcggaggctccaatgagggcctcattacaaccgacaaggt
catcaacaactgcaaggtcgaccagtgccacgctgctgtcaccaaccataagaaatggcagtataacagccctctggtgcctagaaatgcc
gagctgggcgatagaaagggcaagatccatattcccttccctctggccaacgtcacatgtatggtgcccaaggccagaaaccctacagtca
cctacggcaaaaatcaagtgatcatgctgctctaccccgatcatcctacactgctgtcctatagaagcatgggagaagagcccaattaccaa
gaggagtgggtgacccacaaaaaggaggtcgtcctcaccgtgcctacagagggcctcgaggtgacatggggcaataacgagccctaca
agtactggccccagctgtccgctaacggaaccgcccacggccacccccatgagattattctgtactattacgagctgtaccccaccatgacc
gtggtggtcgtgtccgtcgctagctttattctgctgagcatggtgggaatggccgtcggaatgtgcatgtgtgctagaaggaggtgcattaca
ccctatgaactgacacccggagctaccgtcccctttctgctgtctctgatctgctgtattagaacagccaaagccgccacatatcaagaagcc
gccgtctacctctggaatgagcagcagcccctcttttggctgcaagctctgattcccctcgccgctctcatcgtgctgtgcaattgtctgagac
tgctgccttgctgctgcaaaaccctcgcctttctggctgtcatgagcatcggcgctcacaccgtgagcgcttacgagcacgtcaccgtgatcc
ccaacaccgtgggcgtcccctacaagaccctcgtgaatagacccggctacagccctatggtgctggaaatggagctgctgtccgtcacact
ggaacccacactgtccctcgactacatcacatgcgagtacaagaccgtcatcccttccccttacgtgaagtgctgcggcaccgccgagtgc
aaggacaaaaatctccccgactacagctgcaaggtgttcaccggcgtgtatccctttatgtggggaggcgcctattgcttctgcgacgccga
gaatacccaactgtccgaggctcatgtcgagaaatccgagagctgcaaaaccgagttcgccagcgcctacagagcccacacagctagcg
cttccgctaagctgagagtgctgtaccaaggcaataacatcaccgtgaccgcttatgccaacggagatcatgccgtcacagtgaaggacgc
caaattcattgtgggccccatgagctccgcttggacacccttcgataacaagatcgtcgtgtacaagggcgacgtctacaacatggattaccc
cccttttggagctggcagacccggccaattcggagatattcaatctagaacccccgaaagcaaggatgtgtacgccaacacccaactcgtg
ctgcaaaggcccgccgccggaacagtccatgtcccctatagccaagctcctagcggcttcaagtactggctgaaagagaggggagcttct
ctgcagcatacagctcctttcggatgccagatcgccaccaatcccgtgagggccatgaattgtgccgtcggcaacatgcccatttccatcgat
atccccgatgccgctttcacaagggtggtggatgccccctctctgacagacatgagctgtgaagtgcccgcttgtacccacagcagcgactt
cggcggagtcgccattattaagtacgccgtgagcaaaaagggcaagtgtgccgtgcacagcatgaccaatgccgtgaccattagagagg
ccgagatcgaagtcgaaggcaattcccagctccaaatcagctttagcaccgccctcgcctccgctgagttcagagtccaagtgtgcagcac
acaagtgcactgtgctgctgagtgtcatccccccaaagaccacatcgtcaactaccccgctagccacacaacactgggcgtgcaagacatc
tccgctacagccatgagctgggtccaaaagatcaccggaggagtgggactcgtcgtcgctgtcgccgctctgattctgatcgtggtgctctg
tgtgtccttctctagacattga
SEQ ID NO: 6 (CHIKV-E1E2 polyprotein Amino acid Sequence):
MEFIPTQTFYNRRYQPRPWTPRPTIQVIRPRPRPQRQAGQLAQLISAVNKLTMRAVPQQK
PRKNRKNKKQKQKQQAPQNNTNQKKQPPKKKPAQKKKKPGRRERMCMKIENDCIFEV
KHEGKVTGYACLVGDKVMKPAHVKGTIDNADLAKLAFKRSSKYDLECAQIPVHMKSD
ASKFTHEKPEGYYNWHHGAVQYSGGRFTIPTGAGKPGDSGRPIFDNKGRVVAIVLGGA
NEGARTALSVVTWNKDIVTKITPEGAEEWSLAIPVMCLLANTTFPCSQPPCIPCCYEKEP
EETLRMLEDNVMRPGYYQLLQASLTCSPHRQRRSTKDNFNVYKATRPYLAHCPDCGEG
HSCHSPVALERIRNEATDGTLKIQVSLQIGIGTDDSHDWTKLRYMDNHIPADAGRAGLF
VRTSAPCTITGTMGHFILARCPKGETLTVGFTDSRKISHSCTHPFHHDPPVIGREKFHSRP
QHGKELPCSTYVQSNAATAEEIEVHMPPDTPDRTLLSQQSGNVKITVNSQTVRYKCNCG
GSNEGLITTDKVINNCKVDQCHAAVTNHKKWQYNSPLVPRNAELGDRKGKIHIPFPLAN
VTCMVPKARNPTVTYGKNQVIMLLYPDHPTLLSYRSMGEEPNYQEEWVTHKKEVVLT
VPTEGLEVTWGNNEPYKYWPQLSANGTAHGHPHEIILYYYELYPTMTVVVVSVASFILL
SMVGMAVGMCMCARRRCITPYELTPGATVPFLLSLICCIRTAKAATYQEAAVYLWNEQ
QPLFWLQALIPLAALIVLCNCLRLLPCCCKTLAFLAVMSIGAHTVSAYEHVTVIPNTVGV
PYKTLVNRPGYSPMVLEMELLSVTLEPTLSLDYITCEYKTVIPSPYVKCCGTAECKDKNL
PDYSCKVFTGVYPFMWGGAYCFCDAENTQLSEAHVEKSESCKTEFASAYRAHTASASA
KLRVLYQGNNITVTAYANGDHAVTVKDAKFIVGPMSSAWTPFDNKIVVYKGDVYNMD
YPPFGAGRPGQFGDIQSRTPESKDVYANTQLVLQRPAAGTVHVPYSQAPSGFKYWLKE
RGASLQHTAPFGCQIATNPVRAMNCAVGNMPISIDIPDAAFTRVVDAPSLTDMSCEVPA
CTHSSDFGGVAIIKYAVSKKGKCAVHSMTNAVTIREAEIEVEGNSQLQISFSTALASAEF
RVQVCSTQVHCAAECHPPKDHIVNYPASHTTLGVQDISATAMSWVQKITGGVGLVVAV
AALILIVVLCVSFSRH*
SEQ ID NO: 7 (E2 protein Amino Acid Sequence)
STKDNFNVYKATRPYLAHCPDCGEGHSCHSPVALERIRNEATDGTLKIQVSLQIGIGTDD
SHDWTKLRYMDNHIPADAGRAGLFVRTSAPCTITGTMGHFILARCPKGETLTVGFTDSR
KISHSCTHPFHHDPPVIGREKFHSRPQHGKELPCSTYVQSNAATAEEIEVHMPPDTPDRTL
LSQQSGNVKITVNSQTVRYKCNCGGSNEGLITTDKVINNCKVDQCHAAVTNHKKWQY
NSPLVPRNAELGDRKGKIHIPFPLANVTCMVPKARNPTVTYGKNQVIMLLYPDHPTLLS
YRSMGEEPNYQEEWVTHKKEVVLTVPTEGLEVTWGNNEPYKYWPQLSANGTAHGHPH
EIILYYYELYPTMTVVVVSVASFILLSMVGMAVGMCMCARRRCITPYELTPGATVPFLLS
LICCI
SEQ ID NO: 8 (E1 protein Amino Acid Sequence)
RTAKAATYQEAAVYLWNEQQPLFWLQALIPLAALIVLCNCLRLLPCCCKTLAFLAVMSI
GAHTVSAYEHVTVIPNTVGVPYKTLVNRPGYSPMVLEMELLSVTLEPTLSLDYITCEYK
TVIPSPYVKCCGTAECKDKNLPDYSCKVFTGVYPFMWGGAYCFCDAENTQLSEAHVEK
SESCKTEFASAYRAHTASASAKLRVLYQGNNITVTAYANGDHAVTVKDAKFIVGPMSS
AWTPFDNKIVVYKGDVYNMDYPPFGAGRPGQFGDIQSRTPESKDVYANTQLVLQRPAA
GTVHVPYSQAPSGFKYWLKERGASLQHTAPFGCQIATNPVRAMNCAVGNMPISIDIPDA
AFTRVVDAPSLTDMSCEVPACTHSSDFGGVAIIKYAVSKKGKCAVHSMTNAVTIREAEI
EVEGNSQLQISFSTALASAEFRVQVCSTQVHCAAECHPPKDHIVNYPASHTTLGVQDISA
TAMSWVQKITGGVGLVVAVAALILIVVLCVSFSRH
SEQ ID NO: 9 (VSV-G Secretion signal nucleotide sequence):
atgaagtgccttttgtacttagcctttttattcattggggtgaattgc
SEQ ID NO: 10 (VSV-G Secretion signal Amino acid sequence):
MKCLLYLAFLFIGVNC
SEQ ID NO: 11 (VSV-G membrane proximal and TM domain nucleotide sequence):
aatccaatcgagcttgtagaaggttggttcagtagttggaaaagctctattgcctcttttttctttatcatagggttaatcattggactattct
tggttctccgagttggtatccatctttgcattaaattaaagcacaccaagaaaagacagatttatacagacatagagatgaaccgacttggaaa
ggtctga
SEQ ID NO: 12 (VSV-G membrane proximal and TM domain amino acid sequence):
NPIELVEGWFSSWKSSIASFFFIIGLIIGLFLVLRVGIHLCIKLKHTKKRQIYTDIEMNRLGK
V
SEQ ID NO: 13, Protein, Artificial sequence
PKKKRKV
SEQ ID NO: 14, Protein, Artificial Sequence
PKKKRKVEAS
SEQ ID NO: 15, Protein, Artificial Sequence
KRPAATKKAGQAKKKK
SEQ ID NO: 16, Protein, Artificial Sequence
PAAKRVKLD
SEQ ID NO: 17, Protein, Artificial Sequence
RQRRNELKRSP
SEQ ID NO: 18, Protein, Artificial Sequence
NQSSNFGPMKGGNFGGRSSGPYGGGGQYFAKPRNQGGY
SEQ ID NO: 19, Protein, Artificial Sequence
RMRIZFKNKGKDTAELRRRRVEVSVELRKAKKDEQILKRRNV
SEQ ID NO: 20, Protein Artificial Sequence
VSRKRPRP
SEQ ID NO: 21, Protein Artificial Sequence
PPKKARED
SEQ ID NO: 22, Protein, Artificial Sequence
PQPKKKPL
SEQ ID NO: 23, Protein, Artificial Sequence
SALIKKKKKMAP
SEQ ID NO: 24, Protein, Artificial Sequence
DRLRR
SEQ ID NO: 25, Protein, Artificial Sequence
PKQKKRK
SEQ ID NO: 26, Protein, Artificial Sequence
RKLKKKIKKL
SEQ ID NO: 27, Protein, Artificial Sequence
REKKKFLKRR
SEQ ID NO: 28, Protein, Artificial Sequence
KRKGDEVDGVDEVAKKKSKK
SEQ ID NO: 29, Protein, Artificial Sequence
RKCLQAGMNLEARKTKK
SEQ ID NO: 30, Protein, Artificial sequence
MSTDATLIRTTPSHAEADATDTLVATPLMPPRRVISPWPGPGEGQSLMRIPVVDIRGMAL
MPCTPAKARHLLKSGNARPKRNKLGLFYVQLSYEQEPDNQSLVAGVDPGSKFEGLSVV
GTKDTVLNLMVEAPDHVKGAVQTRRTMRRARRQRKWRRPKRFHNRLNRMQRIPPSTR
SRWEAKARIVAHLRTILPFTDVVVEDVQAVTRKGKGGTWNGSFSPVQVGKEHLYRLLR
AMGLTLHLREGWQTKELREQHGLKKTKSKSKQSFESHAVDSWVLAASISGAEHPTCTR
LWYMVPAILHRRQLHRLQASKGGVRKPYGGTRSLGVKRGTLVEHKKYGRCTVGGVDR
KRNTISLHEYRTNTRLTQAAKVETCRVLTWLSWRSWLLRGKRTSSKGKGSHSS
SEQ ID NO: 31, Protein, Artificial sequence
MQPAKQQNWVFQINGDKQPLDMINPGRCRELQNRGKLASFRRFPYVVIQQQTIENPQT
KEYILKIDPGSQWTGFAIQCGNDILFRAELNHRGEAIKFDLVKRAWFRRGRRSRNLRYR
KKRLNRAKPEGWLAPSIRHRVLTVETWIKRFMRYCPIAWIEIEQVRFDTQKLANPEIDGV
EYQQGELQGYEVREYLLQKWGRKCAYCGTENVPLEVEHIQSKSKGGSSRIGNLTLACH
VCNVKKGNLDVRDFLAKSPDILNQVLENSTKPLKDAAAVNSTRYAIVKMAKSICENVK
CSSGARTKMNRVRQGLEKTHSLDAACVGESGASIRVLTDRPLLITCKGHGSRQSIRVNA
SGFPAVKNAKTVFTHIAAGDVVRFTIGKDRKKAQAGTYTARVKTPTPKGFEVLIDGAR
ISLSTMSNVVFVHRSDGYGYEL
SEQ ID NO: 32, Protein, Artificial sequence
MAFVIDKHKRPLMPCSEKRARLLLERGRAVVHRQVPFVIRLKDRTVQHS
AVQPLRVALDPGSRATGMALVREKNTVDTGTGEVYRERIALNLFELVHRG
HRIREQLDQRRNFRRRRRGANLRYRAPRFDNRRRPPGWLAPSLQHRVDTT
MAWVRRLCRWAPASAIGIETVRFDTQRLQNPEISGVEYQQGALAGCEVRE
YLLEKWGRKCAYCGAENVPLEIEHIVPKSRGGSDRVSNLALACRACNQAK
GNRDVRAFLADQPERLARILAQAKAPLKDAAAVNATRWALYRALVDTGLPVEAGTGG
RTKWNRTRLGLPKTHALDALCVGQVDQVRHWRVPVLGIRCAGRGSYRRTRLTRHGFP
RGYLTRNKSAFGFQTGDLIRAVVTKGKKAGTYLGRIAIRASGSFNIQTPMGVVQGIHHR
FCTLLQRADGYGYFVQPKPTEAALSSPRLKAGVSSAGN
SEQ ID NO: 33, Protein, Artificial sequence
MTTNVVFVIDTNQKPLQPCSAAVARKLLLRGKAAMERRYPAVIILKKEVDSVGKPKIEL
RIDPGSKYTGFALVDSKDNADFIIWGTELEHRGAAICKELTKRSAIRRSRRNRKTRYRKK
RFERRKPEGWLAPSLQHRVDTTLTWVKRICKFVPIMSISVEQVKFDLQKLENSDIQGIEY
QQGTLAGYTLREALLEHWGRKCAYCDVENVFLEIEHIYPKSKGGSDKFSNLTLACHKC
NINKGNKSIDEFLLSDHKRLEQIKLHQKKTLKDAAAVNATRKKLVTTLQEKTFLNVLVS
DGASTKMTRLSSSLAKRHWIDAGCVNTTLIVILKTLQPLQVKCNGHGNKQFVTMDAYG
FPRKSYEPKKVRKDWKAGDIIRVTKKDGTMLMGRVKKAAKKLVYIPFGGKEASFSS
ENAKAIHRSDGYRYSFAAIDSELLQKMAT
SEQ ID NO: 34, Protein, Artificial sequence
MPNKYAFVLDSKGKLLDPTKSKKAWYLIRKGKASLVEEYPLIIKLKREVPKDQVNSDKL
ILGIDDGTKKVGFALVQKCQTKNKVLFKAVMEQRQDVSKKMEERRGYRRYRRSHKRY
RPARFDNRSSSKRKGRIPPSILQKKQAILRVVNKLKKYIRIDKIVLEDVSIDIRKLTEGREL
YNWEYQESNRLDENLRKATLYRDDCTCQLCGTTETMLHAHHIMPRRDGGADSIYNLIT
LCKACHKDKVDNNEYQYKDQFLAIIDSKELSDLKSASHVMQGKTWLRDKLSKIAQLEIT
SGGNTANKRIDYEIEKSHSNDAICTTGLLPVDNIDDIKEYYIKPLRKKSKAKIKELKCFRQ
RDLVKYTKRNGETYTGYITSLRIKNNKYNSKVCNFSTLKGKIFRGYGFRNLTLLNRPKG
LMIV
SEQ ID NO: 35, Protein, Artificial sequence
MLFNKCIIISINLDFSNKEKCMTKPYSIGLDIGTNSVGWAVITDNYKVPSKKMKVLGNTS
KKYIKKNLLGVLLFDSGITAEGRRLKRTARRRYTRRRNRILYLQEIFSTEMATLDDAFFQ
RLDDSFLVPDDKRDSKYPIFGNLVEEKVYHDEFPTIYHLRKYLADSTKKADLRLVYLAL
AHMIKYRGHFLIEGEFNSKNNDIQKNFQDFLDTYNAIFESDLSLENSKQLEEIVKDKISKL
EKKDRILKLFPGEKNSGIFSEFLKLIVGNQADFRKCFNLDEKASLHFSKESYDEDLETLLG
YIGDDYSDVFLKAKKLYDAILLSGFLTVTDNETEAPLSSAMIKRYNEHKEDLALLKEYIR
NISLKTYNEVFKDDTKNGYAGYIDGKTNQEDFYVYLKNLLAEFEGADYFLEKIDREDFL
RKQRTFDNGSIPYQIHLQEMRAILDKQAKFYPFLAKNKERIEKILTFRIPYYVGPLARGNS
DFAWSIRKRNEKITPWNFEDVIDKESSAEAFINRMTSFDLYLPEEKVLPKHSLLYETFNV
YNELTKVRFIAESMRDYQFLDSKQKKDIVRLYFKDKRKVTDKDIIEYLHAIYGYDGIELK
GIEKQFNSSLSTYHDLLNIINDKEFLDDSSNEAIIEEIIHTLTIFEDREMIKQRLSKFENIFDK
SVLKKLSRRHYTGWGKLSAKLINGIRDEKSGNTILDYLIDDGISNRNFMQLIHDDALSFK
KKIQKAQIIGDEDKGNIKEVVKSLPGSPAIKKGILQSIKIVDELVKVMGGRKPESIVVEMA
RENQYTNQGKSNSQQRLKRLEKSLKELGSKILKENIPAKLSKIDNNALQNDRLYLYYLQ
NGKDMYTGDDLDIDRLSNYDIDHIIPQAFLKDNSIDNKVLVSSASNRGKSDDFPSLEVVK
KRKTFWYQLLKSKLISQRKFDNLTKAERGGLLPEDKAGFIQRQLVETRQITKHVARLLD
EKFNNKKDENNRAVRTVKIITLKSTLVSQFRKDFELYKVREINDFHHAHDAYLNAVIAS
ALLKKYPKLEPEFVYGDYPKYNSFRERKSATEKVYFYSNIMNIFKKSISLADGRVIERPLI
EVNEETGESVWNKESDLATVRRVLSYPQVNVVKKVEEQNHGLDRGKPKGLFNANLSS
KPKPNSNENLVGAKEYLDPKKYGGYAGISNSFAVLVKGTIEKGAKKKITNVLEFQGISIL
DRINYRKDKLNFLLEKGYKDIELIIELPKYSLFELSDGSRRMLASILSTNNKRGEIHKGNQI
FLSQKFVKLLYHAKRISNTINENHRKYVENHKKEFEELFYYILEFNENYVGAKKNGKLL
NSAFQSWQNHSIDELCSSFIGPTGSERKGLFELTSRGSAADFEFLGVKIPRYRDYTPSSLL
KDATLIHQSVTGLYETRIDLAKLGEG
SEQ ID NO: 36, Protein, Artificial sequence
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
SEQ ID NO: 37, Protein, Artificial sequence
KYSIGLDIGTNSVGWAVITDEYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGY
IDGGASQEEFYKFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSG
EQKKAIVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVD
HIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGD
SEQ ID NO: 38, Protein, Artificial sequence
MDPIRSRTPSPARELLSGPQPDGVQPTADRGVSPPAGGPLDGLPARRTMSRTRLPSPPAPS
PAFSADSFSDLLRQFDPSLFNTSLFDSLPPFGAHHTEAATGEWDEVQSGLRAADAPPPTM
RVAVTAARPPRAKPAPRRRAAQPSDASPAAQVDLRTLGYSQQQQEKIKPKVRSTVAQH
HEALVGHGFTHAHIVALSQHPAALGTVAVKYQDMIAALPEATHEAIVGVGKQWSGAR
ALEALLTVAGELRGPPLQLDTGQLLKIAKRGGVTAVEAVHAWRNALTGAPLN
SEQ ID NO: 39: Protein, Artificial sequence
RPALESIVAQLSRPDPALAALTNDHLVALACLGGRPALDAVKK
GLPHAPALIKRTNRRIPERTSHRVADHAQVVRVLGFFQCHSHP
AQAFDDAMTQFGMSRHGLLQLFRRVGVTELEARSGTLPPASQ
RWDRILQASGMKRAKPSPTSTQTPDQASLHAFADSLERDLDAP
SPMHEGDQTRAS
SEQ ID NO: 40: Protein, Artificial sequence
VITYF
SEQ ID NO: 41, Protein, Artificial sequence
VVVVV
SEQ ID NO: 42, Protein, Artificial sequence
KPWWPRR
SEQ ID NO: 43, Protein, Artificial sequence
NIVNVSLVK
SEQ ID NO: 44, Protein, Artificial sequence
Leu-Pro-Phe-Phe-Asp
SEQ ID NO: 45, Protein, Artificial sequence
KLVF
SEQ ID NO: 46, Protein, Artificial sequence
LVEALYL
SEQ ID NO: 47, Protein, Artificial sequence
RGFFYT

### Example 6: Surface Engineering of Membrane Envelopes for Targeted Delivery

The delivery of molecular cargo to cells represents a major obstacle to the widespread application of gene therapy. While viral vectors and virus-like particles are capable of delivering DNA, RNA, and protein, their cell type specific targeting remains challenging. Explored here are options to manipulate the tropism of viral fusogens by engineering viral fusogens with blunted intrinsic tropism and co-expression of targeting molecules. This platform allows cell type specific targeting using multiple targeting strategies, such as antibodies, single chain antibodies, and ligands, which is name DIRECTED, for Delivery to Intended Recipient Cells Through Envelope Design. It has been shown that DIRECTED can be combined with lentiviral vectors, eVLPs, and SEND, thereby allowing for highly cell type specific delivery of different cargos. Furthermore, it has been shown that viral fusogens from multiple viral families can be harnessed for DIRECTED, opening the door to repeat dosing.

Development of a modular delivery system. Co-expression of protein AG (pAG) together with a viral fusogen (VSIV-G) allows to expand the intrinsic tropism of VSIV-G in the presence of an antibody targeting a surface receptor expressed on target cells (HEK293FT), as shown in FIG. 5A. Blocking of the intrinsic receptor binding capability of VSIV-G by co-incubation with a competitor (dimeric CR2 domain derived from human LDL-R) makes transduction completely dependent on the presence of the antibody, as shown in FIG. 5B. Genetic abolishment of intrinsic tropism of VSIV-G identifies VSIV-G double mutants which produce at ˜50% efficiency of the wildtype version, but show high attenuation of infection, as shown in FIG. 5C. Combining a VSIV-G double mutant (K47Q, R354Q) with pAG results in a highly modular delivery system where tropism can be determined by an antibody targeting cell surface receptors on target cells, as shown in FIG. 5D.

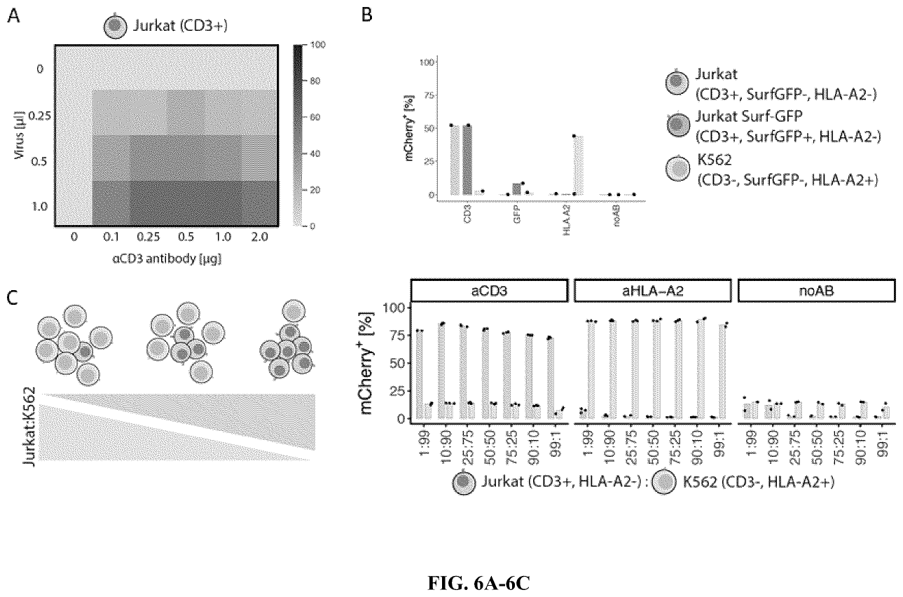

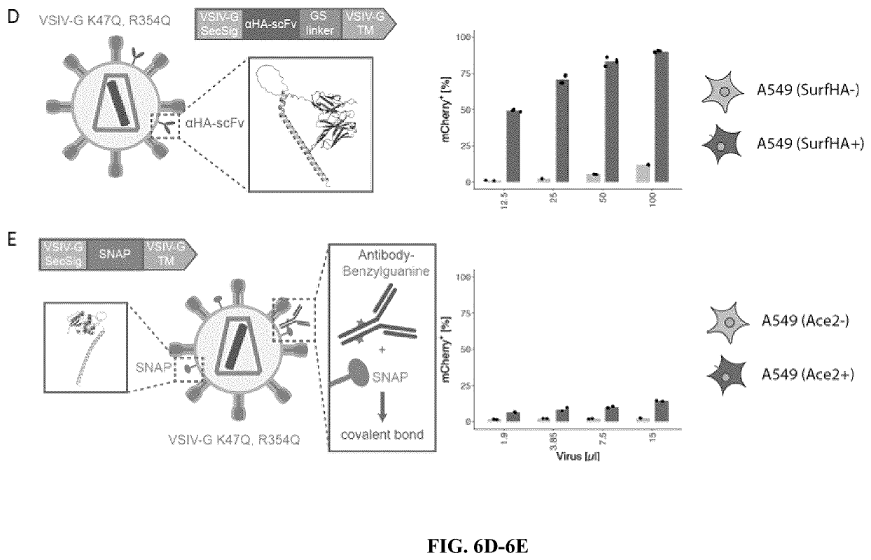

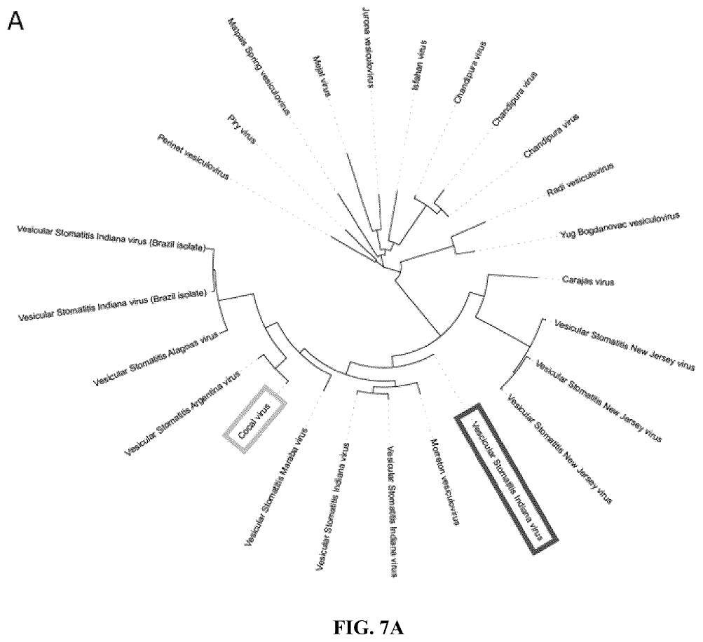

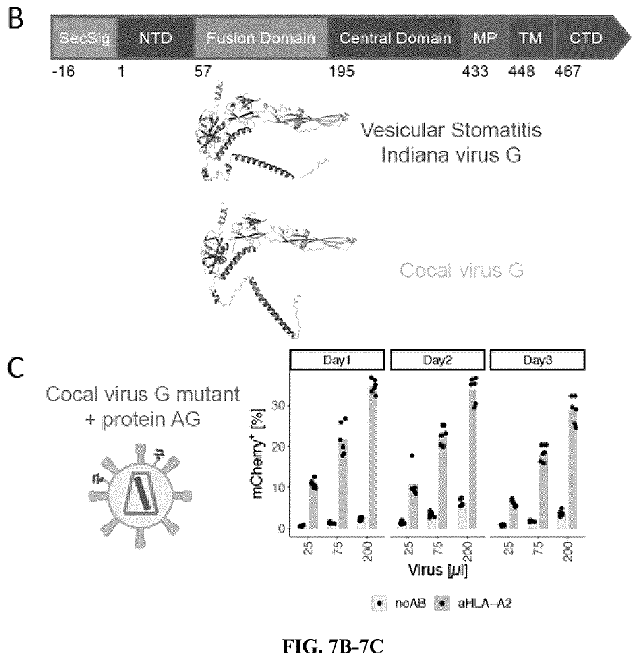

Specificity of DIRECTED. Jurkat T cells were co-incubated with different amounts of DIRECTED-Lentiviral vectors, delivering an H2B-mCherry transgene, and varying amounts of αCD3 antibody. The antibody amount determines the efficiency of cargo delivery, but is robust over a 4-fold range, as shown in FIG. 6A-6B. Co-cultures of Jurkat T cells (CD3+) and K562 cells (HLA-A2+) at different ratios are challenged with DIRECTED-Lentiviral vectors in the presence of an αCD3 antibody, an α-HLA-A2 antibody, or in the absence of antibody and the amount of cells expressing mCherry is determined by Flow cytometry 4 days later. DIRECTED allows targeting of surface marker expressing cells with high efficiency and shows low background in the absence of antibody, as shown in FIG. 6C.

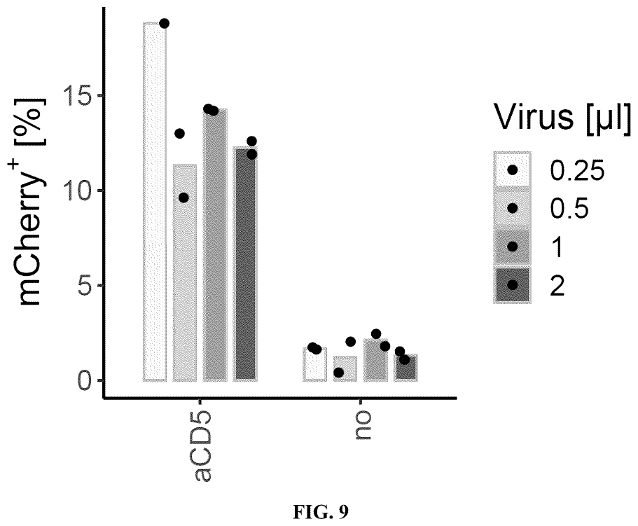

Expansion of targeting strategies. Expression of a membrane-bound single chain variable fragment (scFv) against HA on DIRECTED-Lentiviral vectors enables targeting of and transgene delivery (H2B-mCherry) to cells that express a synthetic Surface HA (SurfHA) receptor, as shown in FIG. 6D. Additionally, a SNAP tag, which interacts with Benzylguanine residues by forming a covalent bond, can be expressed on the surface of enveloped viral vectors and enables targeting of Ace2+ cells after co-incubation with an Anti-Ace2-Benzylguanine antibody, as shown in FIG. 6E. In addition, FIG. 9 shows a protein level readout for H2B-mCherry delivered using a pseudotyped lentiviral vector coexpressing VSV-G (K47Q, R354Q) and a membrane-bound SNAP tag (SNAP-TM) analyzed 3 days after transduction of primary mouse splenocytes. The viral vector preparation was either co-incubated with αCD5-Benzylguanine (against mouse) or with no antibody.

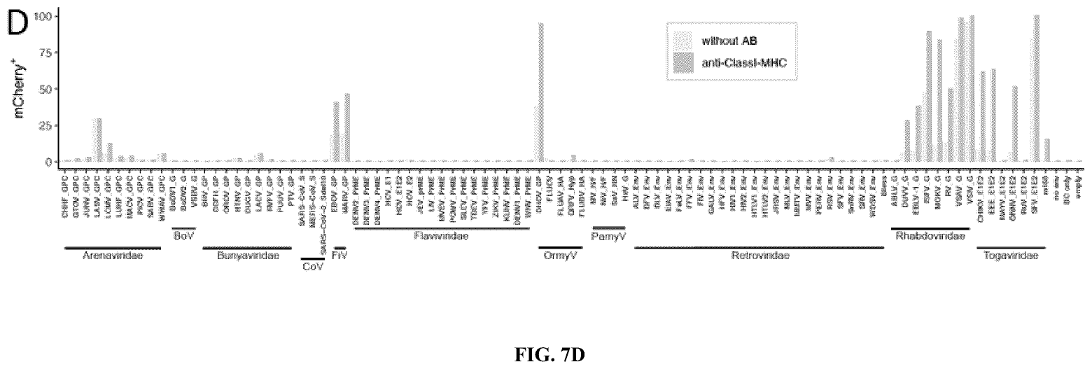

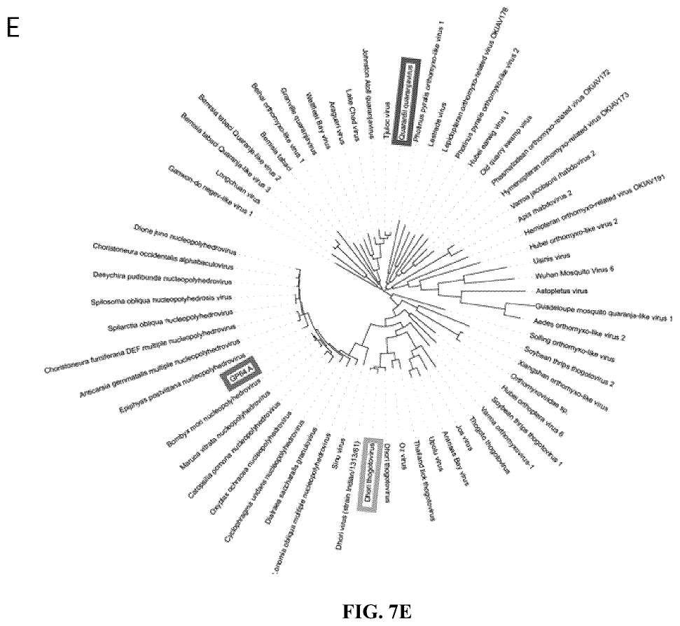

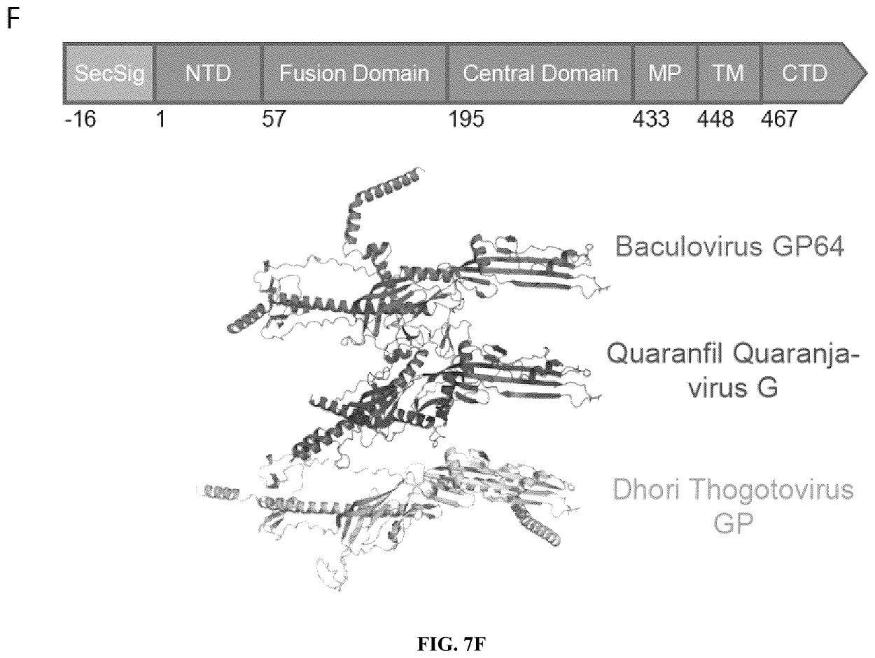

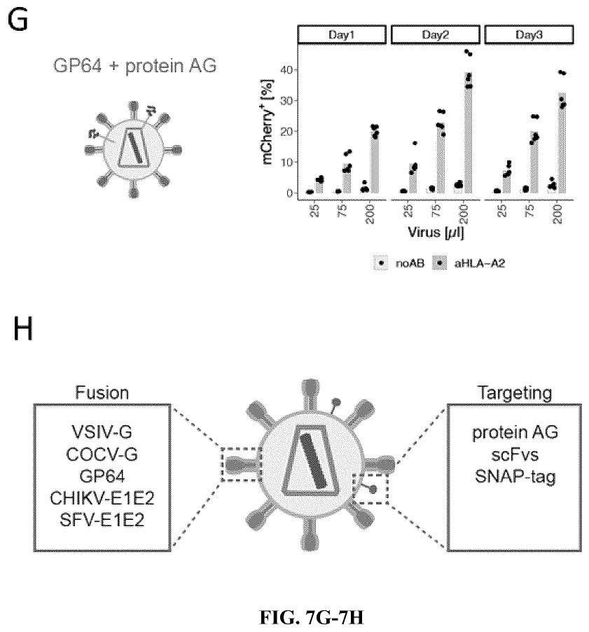

Additional envelopes can be used with DIRECTED. Screening of a library of ˜100 viral fusogens identifies proteins from multiple viral families that can be harnessed for DIRECTED, as shown in FIG. 7D. The families are Filoviridae (FiV), Orthomyxoviridae (OrmyV), Rhabodviridae and Togaviridae. All of these families have been reported to use a pH-dependent uptake mechanism. A sequence-based homology search for Orthomyxoviral envelopes reveals multiple candidates, including the surface protein from Quaranfil quaranjavirus (QRFV) and Dhori thogotovirus (DHOV), which were part of the initial library, as well as baculoviral GP64, as shown in FIG. 7E-7F. Baculoviral GP64 can be effectively redirected in the presence of protein AG (pAG), as shown in FIG. 7G. Further, a sequence-based analysis reveals multiple candidates, including Vesicular Stomatitis Indiana virus G and Cocal virus G, as shown in FIG. 7A-7B. Cocal virus G can be effectively redirected in the presence of protein AG (pAG), as shown in FIG. 7C.

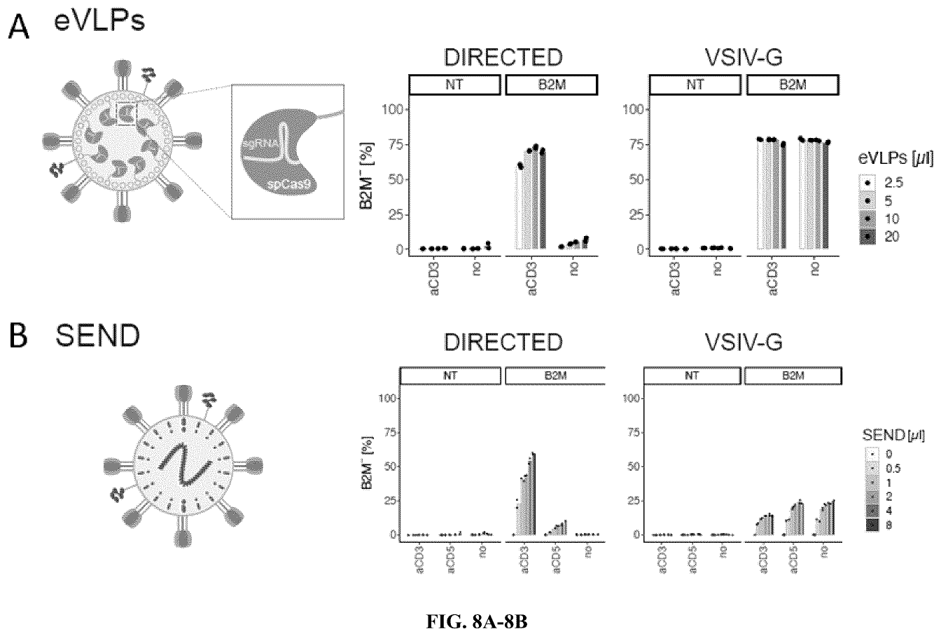

DIRECTED can be combined with eVLPs to deliver RNPs. As shown in FIG. 8A, DIRECTED-eVLPs allow the specific knockout of B2M in Jurkat cells only upon targeting via an Anti-CD3 antibody. Specifically, FIG. 8A shows a protein level readout for Beta-2-Microglobulin (B2M) on protein level by Flow Cytometry after 1 weeks of co-incubating 50,000 Jurkat T cells with eVLPs that package Cas9-sgRNA RNPs with the indicated volume of ˜300× concentrated eVLPs. The particles in the left panel coexpress VSV-G (K47Q, R354Q) and protein AG, and are targeted using the αCD3 or no antibody. The right panel depicts VSV-G wild type.

DIRECTED can be combined with SEND to deliver mRNAs and sgRNAs. As shown in FIG. 8B, DIRECTED-SEND can be used to deliver Cas9 mRNA and sgRNAs to Jurkat T cells in the presence of Anti-CD3 or Anti-CD5 antibodies, but not in the absence of a targeting antibody. Specifically, FIG. 8B shows a protein level readout for Beta-2-Microglobulin (B2M) on protein level by Flow Cytometry after 1 weeks of co-incubating 50.000 Jurkat T cells with SEND particles that package Cas9 mRNA and sgRNA (either non targeting-NT, or B2M targeting-B2M) with the indicated volume of ˜300× concentrated SEND particles. The particles used in the left panel coexpress VSV-G (K47Q, R354Q) and protein AG and are targeted with the indicated antibodies (αCD3, or αCD5, or no antibody). The right hand panel depicts VSV-G wild type.

### Example 7: In Vivo Delivery

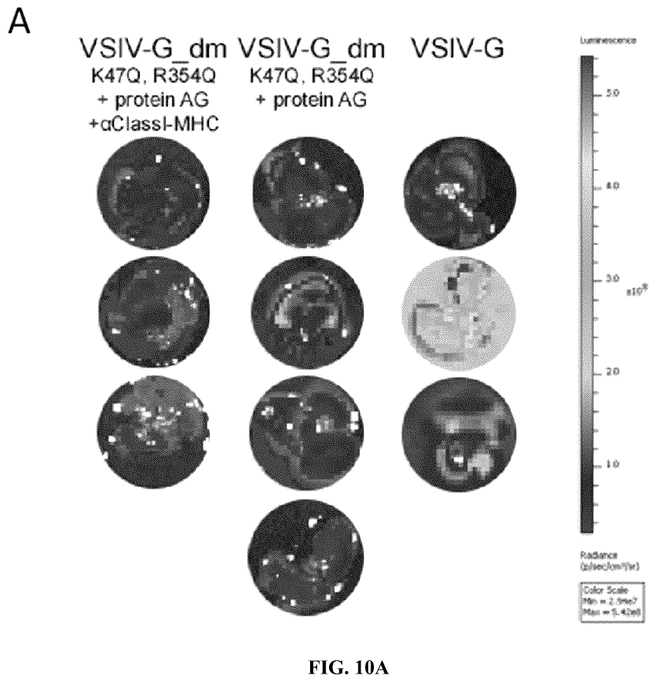

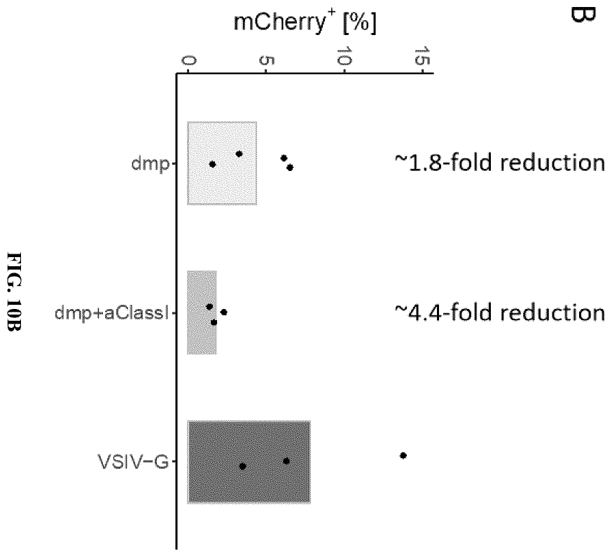

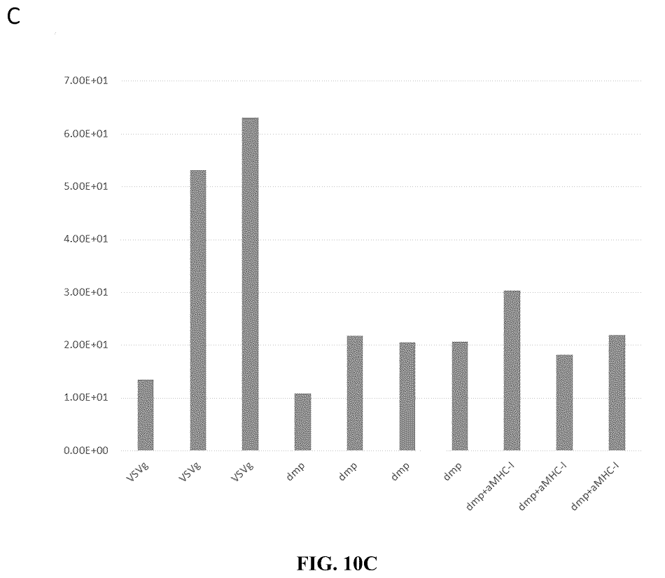

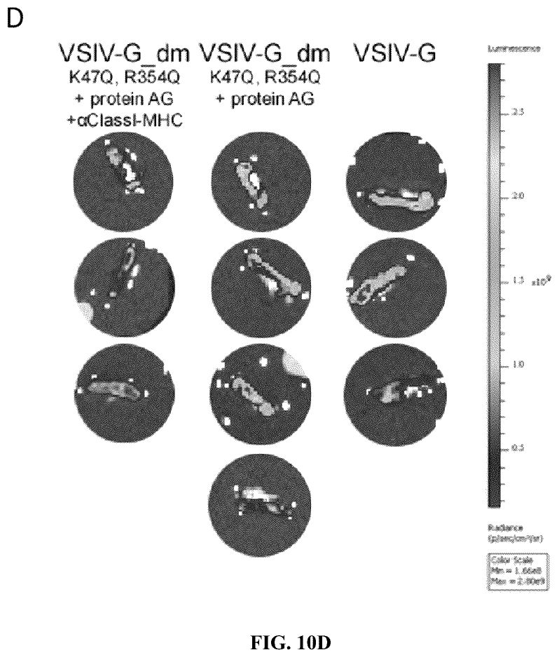

Retro-orbital injection of 3 mice each with VSIV-G, VSIV-G (K47Q, R354Q)+pAG (dmp), or dmp+αMHC-ClassI, was performed at ˜1E11 lentiviral particles per mouse, with H₂B-mCherry-P2A-NanoLuc as the transgene cargo. As shown in FIG. 10A-10C, compared to lentiviral vectors pseudotyped with VSIV-G envelop, lentiviral vectors pseudotyped with dmp and dmp+αMHC-ClassI envelops show 1.8-fold and 4.4-fold reduction in mCherry signals in liver cells, respectively, thereby demonstrating de-targeting of liver with VSIV-G (K47Q, R354Q). As shown in FIG. 10D-10E, compared to lentiviral vectors pseudotyped with VSIV-G envelop, lentiviral vectors pseudotyped with dmp and dmp+αMHC-ClassI envelops show 1.7-fold and 2.9-fold reduction in mCherry signals in spleen B cells, respectively, thereby demonstrating de-targeting of spleen with VSIV-G (K47Q, R354Q). Overall, introduction of K47Q,R354Q mutations reduces transduction of liver cells by ˜2-3-fold as compared to wild type.

Retro-orbital injection of 3 mice each with VSIV-G, VSIV-G (K47Q, R354Q)+SNAP-TM (dmS)+αCD5-BG, are performed with various transgene cargos. Co-delivery of the Benzylguanine-Antibody conjugate and the surface SNAP-tag on the virus allows redirecting of viral particles to specific cell types.

### Example 8: Tissue Targeting

Although the LDL-R receptor used by VSVG is expressed at low levels on photoreceptors (rods and cones), VLDL-R shows intermediate expression levels and is the receptor for alphaviruses (e.g., Semliki Forest Virus, Sindbis Virus). Additional transmembrane proteins that can be targeted include Atp1b2 and Cnga1.

Accordingly, VSIV-G envelop can be redirected to target photoreceptor cells, such as in the form of VSIV-G (K47Q, R354Q)+protein AG (dmp) or VSIV-G (K47Q, R354Q)+SNAP-TM (dmS) and in the presence of receptor-targeting antibodies, via either subretinal injection or intravitreal injection (with low amounts of Pronase E). This approach can be used to deliver a copy of a functional ABCA4 gene or a gene editing system (e.g., CRISPR-Cas9, CRISPR-Cas12, base editor, or prime editor) to correct a mutated ABCA4 gene, for treatment of Stargardt disease. Other diseases associated with photoreceptors and can benefit from the DIRECTED delivery system described herein include Leber congenital amaurosis type 2 (LCA2), Dry Age-related Macular Degeneration (AMD), Wet AMD, Diabetes-Related Macular Edema (DME), Retinitis pigmentosa, Glaucoma, and RGC degeneration associated with neurological decay.

DIRECTED can also be used to target muscle cells in vivo. For example, VSIV-G envelop can be redirected to target muscle cells, such as in the form of VSIV-G (K47Q, R354Q)+protein AG (dmp) or VSIV-G (K47Q, R354Q)+SNAP-TM (dmS) and in the presence of anti-Integrin-αVβVI antibodies.

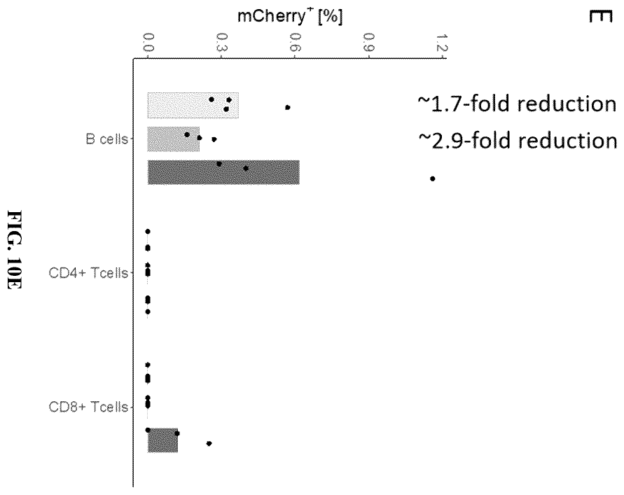

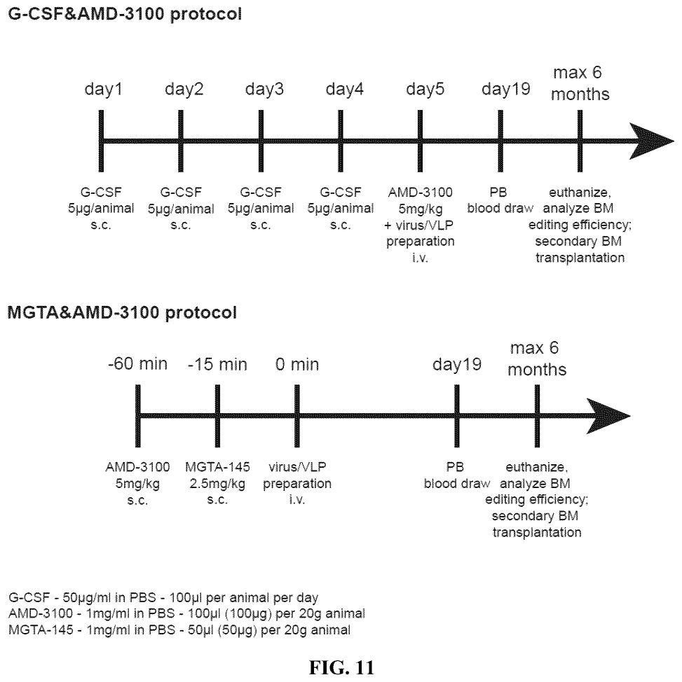

DIRECTED can also be used to target eVLPs to HSCs in vivo. Optionally, the HSCs can be mobilized with G-CSF (s.c., 5 mg/mouse/day, 4 days), followed by s.c. injection of AMD3100 (Plerixafor, 5 mg/kg) on day 5. In addition, dexamethasone (10 mg/kg) i.p. at 16 h and 2 h before virus injection can help to mobilize more HSCs. An exemplary protol includes treating mice with G-CSF (s.c., 5 mg/mouse/day, 4 days), and co-injecting virus (at least 1E10 VGs) with AMD3100 (5 mg/kg) via tail vein, with AMD3100 showing maximal mobilization 1 hour after injection. See FIG. 11. Such eVLPs targeting HSCs can be used for treatment of sickle-cell anemia caused by mutation in hemoglobin gene, such as by converting HBBS mutation to HBBG using ABE8e-NRCH base editor delivered as RNPs.

### Example 9

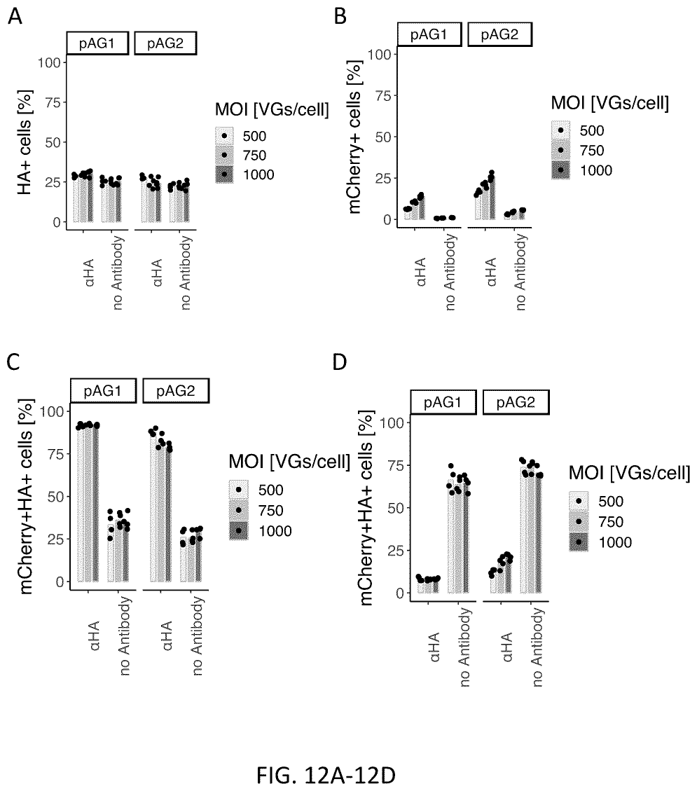

Mixing of target and non-target cells. Surface-HA HEK293FT cells (target cells) were mixed with HEK293FT cells (non-target cells). (FIG. 12A) Two preparation of VSV-G K47Q, R354Q+protein AG (pAG) virus were produced and titrated using RT-qPCR for viral genomes (VGs). Incubated with two preparations of VSV-G K47Q,R354Q+protein AG (pAG) virus in presence or absence of targeting antibody at different multiplicities of infection (MOIs; 500, 750, 1000 VGs/cell). Approximately 25% of cells were Surface-HA+ (target cells) in all conditions as determined by flow cytometry after staining with αHA-PB450. Overall infection of cells was determined by Flow cytometry (by detecting the H2B-mCherry transgene that was delivered) (FIG. 12B). Presence of antibody shows a boost of cells that get infected over no antibody pAG2 preparation shows higher transduction efficiency overall. Analysis of the percentage of successfully transduced target cells in presence of αHA antibody reveals high percentage of target cells transduced (FIGS. 12C-12D). In the absence of an antibody the frequency of target cells is about 25%, which would be expected due to the frequency with which target cells are represented in the population. Of the ˜1% of cells that were transduced at MOI 1000 in the absence of antibody 70-75% were non-target cells and 25-30% were Surface-HA+ (target cells).

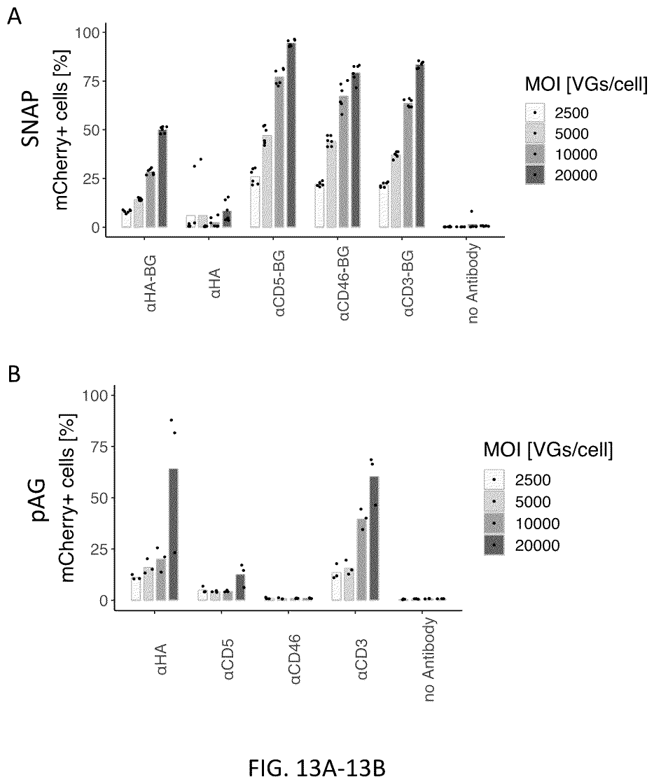

Comparison of SNAP and proteinAG (pAG) strategy for targeting of different receptors expressed on Surface-HA. Jurkat cells. SNAP shows high transduction efficiency in the presence of αHA-BG, αCD5-BG, αCD46-BG, and αCD3-BG (FIG. 13A). As expected, αHA without benzylguanine does not increase transduction. Similarly, absence of any antibody does not result in successful infection of Surface-HA+Jurkat cells. pAG allows efficient transduction of Surface-HA+Jurkat cells with αHA, and αCD3 (FIG. 13B). However, αCD5 and αCD46 do not result in efficient infection of Surface-HA+Jurkat cells. This may be caused by different turnover rates of the target receptors. While SNAP results in covalent immobilization of antibody-BG substrates on virions, the pAG strategy relies on protein-protein interactions, which are intrinsically more transient. SNAP outperforms pAG at same antibody concentration used (375 ng Antibody per 15.000 cells). Expression level of surface receptors is not main indicator of pAG efficiency. Possibly, CD5 and CD46 have a slow turnover, which doesn't allow pAG virus to interact with antibody for long enough before internalization.

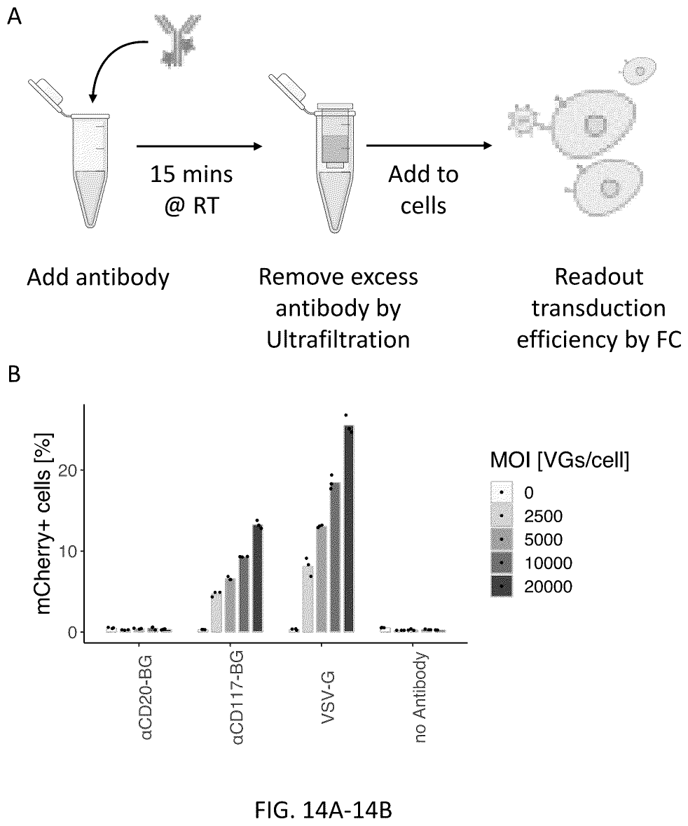

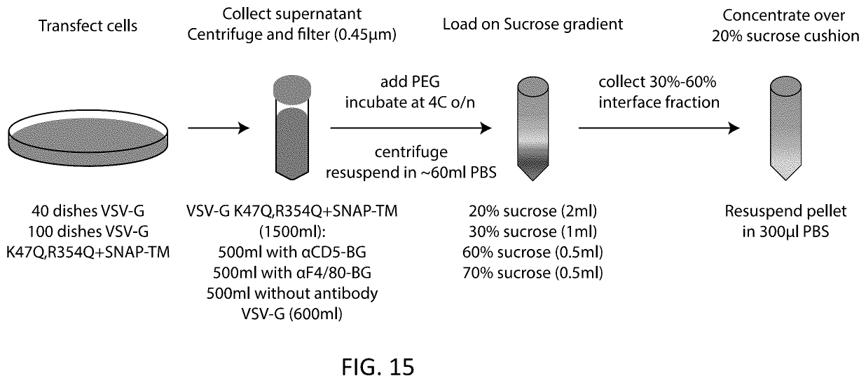

Targeting of CD117 (c-Kit) on Kasumi-1 cells. FIG. 14A shows the experimental setup: Viral particles were incubated with antibody-BG for 15 minutes at room temperature, before excess antibody-BG was removed by ultrafiltration (100 kDa cutoff) by washing for 3 times with an excess of PBS. FIG. 14B shows the results of the experiment. CD117 (c-Kit) is a receptor that is highly expressed on hematopoietic stem cells (HSCs), therefore representing an attractive target for gene delivery to HSCs. Using CD117+Kasumi-1 cells we could show that co-incubation of VSV-G mutant K47Q, R354Q+SNAP particles with αCD117-BG result in efficient transduction of these cells, to levels similar to VSV-G. αCD20-BG and the absence of antibody show no successful transduction as evaluated by Flow cytometry for the H2B-mCherry transgene.

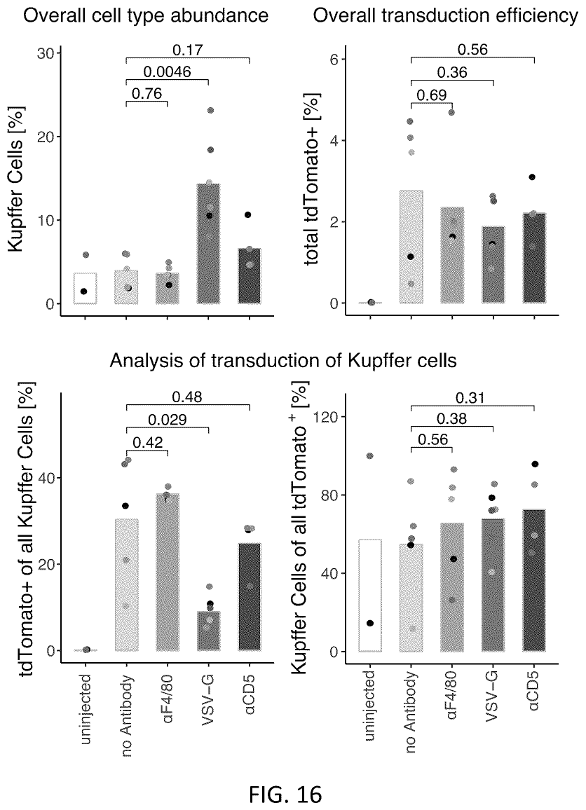

Analysis of transduced cells in vivo in liver Kupffer cells. CreVLP preps were normalized to functional Cre amount (which correlated very well with overall Cre content of CreVLPs). Animals were injected with 50 μl of CreVLPs via tail-vein. VSV-G (n=6), dmS (no Antibody; n=5), dmS+αCD5 (n=4), and dmS+αF4/80 (n=5). As a result, in animals injected with VSV-G, an increase in macrophages was observed. Overall a similar amount of cells were transduced. Percentage of Kupffer cells of all tdTomato+ cells was ˜50-60%. (FIG. 16). All variants were observed to cause infection of Kupffer cells with high efficiency. However, only VSV-G causes an increase in Kupffer cells, which could be indicative of an immune response. Most of the virus seems to be taken up by macrophages.

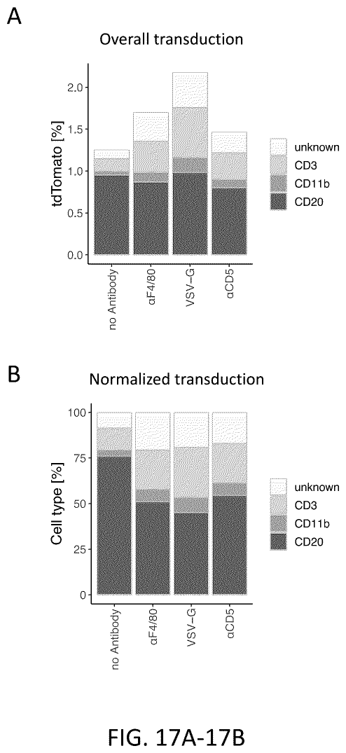

Analysis of transduced cells in vivo in spleen cells. FIG. 17A shows overall transduction. FIG. 17B shows normalized transduction. Overall similar transduction efficiencies were observed in all conditions. Majority of transduced cells are CD20+ (B cells)

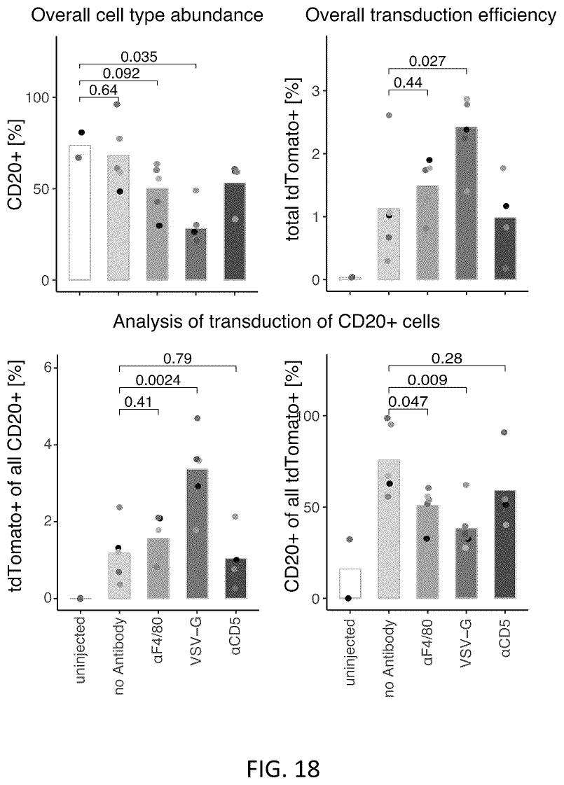

Analysis of transduced cells in vivo—Spleen CD20+ cells. CD20 relative cell abundance was reduced in VSV-G condition (FIG. 18). Overall a similar amount of cells was transduced. Around 50-70% of transduced cells are CD20+. CD20+ cells are the most efficiently transduced in all conditions. Decrease in CD20+ cells in spleens could indicate inflammation in VSV-G condition.

1-57. (canceled)

58. A targeted delivery vehicle comprising:
a lipid bilayer membrane, wherein the lipid bilayer membrane forms a vesicle;
a fusogen embedded in the lipid bilayer membrane;
a targeting moiety embedded in the lipid bilayer membrane, wherein the targeting moiety is separate and different from the fusogen; and
a cargo within the vesicle.

59. The targeted delivery vehicle of claim 58, wherein the fusogen is an envelope protein from a virus.

60. The targeted delivery vehicle of claim 59, wherein the envelope protein is modified to not have a targeting function.

61. The targeted delivery vehicle of claim 59, wherein the virus is selected from the group consisting of genera Arenaviridae, Filoviridae, Orthomyxoviridae, Rhabdoviridae, Togaviridae, Matonaviridae, Hantaviridae, Bunyaviridae, Retroviridae, Coronaviridae, Bornaviridae and Orthomyxoviridae.

62. The targeted delivery vehicle of claim 59, wherein the virus is selected from the group consisting of Pichinde virus, Ebola virus, Dhori virus, Duvenhage lyssavirus, European bat 1 lyssavirus, Isfahan virus, Mokola virus, Rabies virus, Chikungunya virus, Eastern equine encephalitis virus, O'nyong'nyong virus, Rubella virus, Hantaan orthohantavirus, Dugbe virus, La Crosse virus, Influenza A virus, Quaranfil virus, Lassa mammarenavirus, Lymphocytic Choriomeningitis virus, Mammalian Bornavirus 1, Marburg virus, Feline immunodeficiency virus, Rabies virus, Arizona vesiculovirus, Eastern equine encephalitis virus, Semliki Forest virus, Hantaan orthohantavirus, Indiana vesiculovirus, Severe acute respiratory syndrome coronavirus, Severe acute respiratory syndrome coronavirus 2, Influenza A virus, Baboon endogenous virus, Vesicular Stomatitis Virus and Sindbis virus.

63. The targeted delivery vehicle of claim 58, wherein the fusogen is a pH-dependent fusogen.

64. The targeted delivery vehicle of claim 63, wherein the pH-dependent fusogen is selected from the group consisting of Sindbis Virus E2 protein, Vesicular Stomatitis Virus G protein, Cocal Virus G protein, and Chikungunya Virus E2 protein.

65. The targeted delivery vehicle of claim 64, wherein:
(a) the fusogen is Vesicular Stomatitis Virus G (VSV-G) protein, and the VSV-G protein comprises at least one nonconservative point mutation at a position selected from the group consisting of H8, K47, Y209, and R354 or comprises at least one mutation selected from the group consisting of H8A, K47Q, Y209A, and R354Q; or
(b) the fusogen is Cocal Virus G protein, and the Cocal Virus G protein comprises at least one nonconservative point mutation at a position selected from the group consisting of Q25, K64, Y226, and R371 or comprises at least one mutation selected from the group consisting of Q25A, K64Q, Y226A, and R371Q; or
(c) the fusogen is Chikungunya Virus E2 protein, and the Chikungunya Virus E2 protein comprises at least one nonconservative point mutation at a position selected from the group consisting of W64, D71, T116, 1121, 1190, Y199, and I217 or comprises at least one mutation selected from the group consisting of D71A, 1121A, 1190A, Y199A, and I217A.

66. The targeted delivery vehicle of claim 58, wherein the fusogen comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM); and/or wherein the targeting moiety comprises a transmembrane domain selected from the group consisting of a Vesicular Stomatitis Virus G C terminal domain (VSVG-CTD), a transmembrane domain of B2M, a transmembrane domain of HLA-A, and a transmembrane domain of platelet derived growth factor receptor beta (PDGFRB-TM).

67. The targeted delivery vehicle of claim 58, wherein the targeting moiety comprises a binding domain specific for a target cell of interest.

68. The targeted delivery vehicle of claim 67, wherein the binding domain comprises a receptor, an antibody, or an antigen-binding fragment; wherein the antigen-binding fragment is selected from the group consisting of a Fab, a Fab′, a F(ab′)₂, an Fd, an Fv, a domain antibody, a complementarity determining region (CDR), a single chain variable fragment antibody (scFv), a maxibody, a minibody, an intrabody, a diabody, a triabody, a tetrabody, a v-NAR and a bis-scFv.

69. The targeted delivery vehicle of claim 58, wherein the targeting moiety comprises a tag, wherein the tag is selected from the group consisting of a SNAP tag, a biotin tag, an Isopeptag, a SpyTag, a SpyCatcher tag, a SnoopTag, a SnoopTagJr, a SnoopCatcher tag, a DogTag, a DogCatcher tag, a Gluthatione-S-transferase tag, a CLIP tag, a Protein A tag, a Protein G tag, a Protein AG tag, a GFP tag, an HA tag, a FLAG tag and a HiBiT-tag.

70. The targeted delivery vehicle of claim 58, wherein the target cell of interest is a B cell, a CD4+ T cell, a CD8+ T cell, a lung cell, a colorectal cell, a hematopoietic stem cell, a muscle cell, a cardiac cell, a hepatocyte, a monocyte, a macrophage, a neuronal cell, or a cancel cell.

71. The targeted delivery vehicle of claim 58, wherein the cargo comprises a deoxyribonucleic acid (DNA), a ribonucleic acid (RNA), a protein, a ribonucleoprotein (RNP), or a combination thereof.

72. The targeted delivery vehicle of claim 58, wherein the targeted delivery vehicle is a viral-like particle, an extracellular vesicle, a synthetic liposome, an enveloped virus, a pseudotyped lentiviral vector, a selective endogenous encapsidation for cellular delivery system (SEND), a nanoblade or a gesicle.

73. A method for targeted delivery of a cargo comprising administering a targeted delivery vehicle to a subject in need of the cargo, wherein the targeted delivery vehicle comprises:
a lipid bilayer membrane, wherein the lipid bilayer membrane forms a vesicle;
a fusogen embedded in the lipid bilayer membrane;
a targeting moiety embedded in the lipid bilayer membrane, wherein the targeting moiety is separate and different from the fusogen; and
a cargo within the vesicle.

74. A vector system for producing the targeted delivery vehicle of claim 58, wherein the vector system comprises one or more vectors encoding the fusogen and the targeting moiety.

75. A host cell comprising or transformed with the vector system of claim 74.

76. A host cell for producing the targeted delivery vehicle of claim 58, wherein the host cell comprising one or more polynucleotides encoding the fusogen and the targeting moiety.

77. A method for producing the targeted delivery vehicle of claim 58, comprising expressing one or more polynucleotides encoding the fusogen and the targeting moiety in a host cell in the presence of the cargo.
